(19) United States
(12) Patent Application Publication
(10) Pub. No.: US 2020/0231630 A1
(43) Pub. Date: Jul. 23, 2020

(54) MOLECULAR GRAFTING OF COMPLEX, BROADLY NEUTRALIZING ANTIBODY EPITOPES

(71) Applicant: THE CHILDREN'S MEDICAL CENTER CORPORATION, Boston, MA (US)

(72) Inventors: Aaron G. SCHMIDT, Boston, MA (US); Goran BAJIC, Boston, MA (US); Max J. MARON, Brookline, MA (US)

(73) Assignee: THE CHILDREN'S MEDICAL CENTER CORPORATION, Boston, MA (US)

(21) Appl. No.: 16/648,475

(22) PCT Filed: Sep. 28, 2018
(86) PCT No.: PCT/US2018/053402

§ 371 Date: Mar. 18, 2020

Related U.S. Application Data
(60) Provisional application No. 62/564,775, filed on Sep. 28, 2017.

(51) Int. Cl. C07K 14/005; C12N 7/00; A61K 39/145

(52) U.S. Cl. CPC C07K 14/005; C12N 7/00; A61K 2039/552; C12N 2760/16122; A61K 39/145

(57) ABSTRACT

Described herein is a chimeric epitope comprising (a) a conserved donor receptor binding site (RBS) or a neutralizing epitope, or a functional fraction thereof, and (b) an acceptor molecular scaffold or fragment thereof. Another aspect provides a chimeric epitope comprising (a) a conserved donor receptor binding site (RBS) derived from circulating H1 influenza, and (b) an acceptor molecular scaffold derived from non-circulating influenza. Further, compositions and methods for inducing an immune response and vaccination are described herein.

### CROSS-REFERENCE TO RELATED APPLICATION

This Application claims benefit under 35 U.S.C. § 119(e) of the U.S. Provisional Application No 62/564,775 filed Sep. 28, 2017, the contents of which is incorporated herein by reference in its entirety.

### GOVERNMENT SUPPORT

This invention was made with Government support under Grant No. P01 A1089618 and U19 AI-059937 awarded by the National Institutes of Health. The Government has certain rights in the invention.

### FIELD OF THE INVENTION

The field of the invention relates to methods for making an influenza vaccine, and uses thereof.

### BACKGROUND

Pathogens like influenza evade host immune surveillance by rapidly evolving their surface-exposed glycoproteins to change their antigenicity. Influenza evolves primarily at the human population level and within its animal reservoirs (swine and avian). In response to host humoral pressure, which predominantly targets the viral hemagglutinin (HA), the virus mutates, rendering previous immune responses to HA suboptimal. The humoral response then evolves to refine previous responses to recognize the mutated viruses. The net effect of this on-going selection across the entire population is a virus-immunity “arms race”. This antigenic variation and subsequent co-evolution of influenza with the human population has resulted in often ineffective influenza vaccines. Nevertheless, the most effective protection against influenza continues to be seasonal vaccination. Since 1977, H1 and H3 influenza A viruses have co-circulated in the human population along with two influenza B lineages. The current seasonal vaccine requires predicting the newly drifted H1, H3 and B strains for the upcoming year. This prediction, however, is extremely variable from year-to-year and ranges in effectiveness from 10-60%. Furthermore, the current seasonal vaccine cannot protect against novel, pandemic influenzas. New approaches to rational vaccine design are necessary to create a universal or broadly-protective influenza vaccine. Such a vaccine should induce broad immunity a) within seasonal, circulating H1, H3 and B influenzas, b) across subtypes (heterosubtypic) and c) to pre-pandemic influenzas.

Further complicating the development of a broadly-protective vaccine is the preexisting, anti-influenza immunity within the human population. Initial influenza exposure whether through vaccination or infection profoundly influences subsequent immune responses. This immunological memory or “Original Antigenic Sin” (14, 15) predominantly results in updating memory B-cell responses that lead to refined, strain-specific responses. A broadly-protective influenza vaccine thus requires immunogen(s) that can redirect preexisting humoral responses to convert these strain-specific responses into broadly neutralizing responses.

Current vaccine strategies have predominantly focused on eliciting broadly neutralizing antibodies (bnAbs) to conserved sites on the viral HA. The two relatively invariant epitopes so far recognized are the receptor binding site (RBS) on the HA “head” and a surface along the HA “stem.” Analyses of antibody repertoires after influenza exposure show that these conserved epitopes are often subdominant while the epitopes outside the RBS in the globular head are immunodominant. Thus, immunogen design strategies must aim to change the patterns of immunodominance. Three major design strategies aim to elicit stem-directed bnAbs by modifying the immunodominant head through i) hyperglycosylation, ii) removal of the head to create stem-only mini-constructs, and iii) a “cut-and-paste” approach to swap HA heads from non-circulating HAs onto conserved, circulating stems resulting in “chimeric” HAs. A significant problem, however, with stem-based vaccines is that the majority of the elicited responses are non-neutralizing and thus fail to actually prevent infection; their mechanism of action is largely ADCC-mediated. Furthermore, the vast majority of stem-directed bnAbs are genetically restricted to the VH1˜69 gene family. This gene usage is associated with auto- and polyreactivity which may prove a significant hurdle for eliciting bnAbs in the human population due to tolerance mechanisms. While stem-directed bnAbs are undoubtedly a useful component of a broadly-protective vaccine, strategies to elicit bnAbs targeting the conserved RBS epitope are necessary.

One approach towards eliciting head-directed responses are the Computationally Optimized Broadly Reactive Antigens or “COBRA” immunogens. This approach obtains a consensus sequence from antigenic variants of circulating influenzas to create an “optimized” HA protein that can induce robust hemagglutination inhibition (HAI) activity and thus prevents infection. However, this strategy has not specifically shown that the conserved RBS is the target of the protective responses as many head-directed Abs can have HAI activity but do not specifically interact with the RBS.

### SUMMARY

Provided herein are compositions comprising an antigenic fusion protein that can be used to raise an immune response in a host that is not strain-specific, thus providing immunity against multiple viral subtypes (e.g., influenza A and/or B subtypes) or can provide long-term immunity. A strain-independent immune response is possible because the antigenic fusion protein comprises a chimeric epitope that is directed to a conserved region of a given virus. This conserved region is not typically subject to antigenic drift and is shared amongst other antigenically distinct viruses.

One aspect of the invention described herein relates to a chimeric epitope comprising, at least one conserved donor receptor binding site (RBS) or neutralizing epitope (e.g., at least 1, 2, or 3), or a functional fraction thereof; and an acceptor molecular scaffold or fragment thereof. In one embodiment, the RBS or the neutralizing epitopes are fused to the molecular scaffold. In one embodiment, the chimeric epitope comprises at least two conserved donor RBS. Such conserved donor RBSes can be the same or different. For example, a trimeric donor can comprise at least two or at least three different conserved RBS from distinct or antigenically distinct viral donors. Alternatively, all three RBSes can be from the same donor.

In one embodiment of any aspects described herein, the RBS is a conserved epitope on the influenza virus hemagglutinin (HA).

In one embodiment of any aspects described herein, the acceptor molecular scaffold is an antigenically distinct HA. In one embodiment of any aspects described herein, the HA comprises an HA without the RBS or the neutralizing epitopes.

In one embodiment of any aspects described herein, the donor RBS or the neutralizing epitope and the acceptor molecular scaffold are derived from a family of viruses selected from the group consisting of: Arenaviridae, Bunyaviridae, Coronaviridae, Filoviridae, Flaviviridae, Orthomyxoviridae, Paramyxoviridae, Pneumoviridae, and Retroviridae. In one embodiment of any aspects described herein, the donor RBS and the acceptor molecular scaffold are derived from the family Orthomyxoviridae.

In one embodiment of any aspects described herein, donor RBS and the acceptor molecular scaffold are derived from the same viral family. In one embodiment of any aspects described herein, the donor RBS and the acceptor molecular scaffold are derived from a different viral family.

In one embodiment of any aspects described herein, wherein the donor RBS and the acceptor molecular scaffold are derived from the same viral family but different antigenic viral types.

In one embodiment of any aspects described herein, the donor RBS and the acceptor molecular scaffold are derived from the same viral family but different host of origin.

In one embodiment of any aspects described herein, the donor RBS and the acceptor molecular scaffold are derived from the same viral family but different geographical origin.

In one embodiment of any aspects described herein, the donor RBS and the acceptor molecular scaffold are derived from the same viral family but different viral strains or subtypes.

In one embodiment of any aspects described herein, the donor RBS and the acceptor molecular scaffold are derived from the same viral family but different year of isolation.

In one embodiment of any aspects described herein, the donor RBS is the RBS of circulating, previously circulating, or pre-pandemic influenzas viruses. Non-limiting examples of circulating or previously circulating influenzas include H1, H2, H3 or B. Non-limiting examples of pre-pandemic influenza viruses include H5, H7 and H9 influenzas.

In one embodiment of any aspects described herein, the RBS is an RBS of H1 influenza was isolated in 1918-present day. In one embodiment of any aspects described herein, the RBS of H1 influenza is H1/Massachusetts/1/1990; H1/Solomon Islands/3/2006; or H1/California/04/2009 or a variant thereof.

In one embodiment of any aspects described herein, the molecular scaffold has substantially no preexisting immunity in the population of a subject. In one embodiment of any aspects described herein, the molecular scaffold does not boost a strain-specific response.

In one embodiment of any aspects described herein, the molecular scaffold is derived from H2, H4, H5, H6, H7, H8, H9, H10, H11, H12, H13, H14, H15, H16, H17 or H18 influenzas.

In one embodiment of any aspects described herein, the molecular scaffold is derived from group 1 influenzas or group 2 influenzas. Non-limiting examples of group 1 influenza include H2N2 A/Japan/305/1957; H5N8 A/gyrfalcon/Washington/41088-6/2014; H6N8 A/widgeon/Wisconsin/617/1983; H9N2 A/swine/Hong Kong/9/1998; and H16N3 A/laughing-gull/Delaware Bay/296/1998. Non-limiting examples of group 2 influenza include H3N2 A/Aichi/2/1968; H4N6 A/America black duck/New Brunswick/00464/2010; H7N9 A/Shanghai/1/2013; H10N7 A/mallard/Wisconsin/1350/1983; and H14N6 A/mallard/Wisconsin/10OS3941/2010.

Another aspect of the invention described herein relates to a chimeric epitope comprising a conserved donor receptor binding site (RBS) derived from circulating H1 influenza and an acceptor molecular scaffold derived from non-circulating influenza.

In one embodiment of any aspects described herein, the molecular scaffold is engineered to comprise at least one amino acid mutation. Non-limiting examples of the at least one amino acid mutation include N145S, T192R, S193A, K196H, A198E and S219K.

Another aspect of the invention described herein relates to an immunogenic composition comprising any chimeric epitope described herein and a pharmaceutically acceptable carrier.

In one embodiment of any aspects described herein, the composition is used to elicit an immune response in a subject.

In one embodiment of any aspects described herein, the composition is used for a diagnostic for exposure to a pathogen or immune threat.

In one embodiment of any aspects described herein, the composition is used to prevent an infection caused by a pathogen in a subject. In one embodiment of any aspects described herein, the infection is an influenza infection.

In one embodiment of any aspects described herein, the composition for the use of vaccinating a subject.

Another aspect of the invention described herein relates to a method for inducing an immune response in a subject by administering to a subject any chimeric epitope described herein.

Another aspect of the invention described herein relates to a method for vaccinating a subject by administering to a subject any chimeric epitope described herein.

Another aspect of the invention described herein relates to a method for inducing an immune response in a subject by administering to a subject the immunogenic composition described herein.

Another aspect of the invention described herein relates to a method for vaccinating a subject by administering to a subject the immunogenic composition described herein.

In one embodiment of any aspects described herein, the subject is human. In one embodiment of any aspects described herein, the subject is an agricultural or non-domestic animal. In one embodiment of any aspects described herein, the subject is a domestic animal. In one embodiment of any aspects described herein, the subject is a bird.

In one embodiment of any aspects described herein, the RBS and the molecular scaffold are a mammalian RBS and the molecular scaffold. In one embodiment of any aspects described herein, the RBS and the molecular scaffold are a human RBS and the molecular scaffold. In one embodiment of any aspects described herein, the RBS and the molecular scaffold are a bird RBS and the molecular scaffold.

### Definitions

As used herein, the terms “preventing” and “prevention” have their ordinary and customary meanings, and include one or more of: preventing the growth or the increase in the growth of a population of a virus or pathogen in a subject, preventing development of a disease caused by a virus or pathogen in a subject, for example, influenza infection; and preventing symptoms of an infection or disease caused by a virus or pathogen infection in a subject. As used herein, the prevention lasts at least about 0.5 days, 1 day, 5 days, 15 days, 30 days, 1 month, 6 months, 1 year, 5 years, 10 years, or more after administration or application of the effective amount of the chimeric epitope, as described herein. In one embodiment, prevent refers to: (i) the prevention of infection or re-infection, as in a traditional vaccine, (ii) the reduction in the severity of, or, in the elimination of symptoms, and (iii) the substantial or complete elimination of the pathogen or disorder in question.

The “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation, for example the carrier does not decrease the impact of the agent on the treatment. In other words, a carrier is pharmaceutically inert. The terms “physiologically tolerable carriers” and “biocompatible delivery vehicles” are used interchangeably.

The term “administered” is used interchangeably in the context of treatment of a disease or disorder. Both terms refer to a subject being treated with an effective dose of a chimeric epitope or an immunogenic composition comprising a chimeric epitope of the invention by methods of administration, for example subcutaneous or systemic administration.

The phrases “systemic administration,” “administered systemically”, “peripheral administration” and “administered peripherally” as used herein mean the administration of a chimeric epitope or an immunogenic composition comprising a chimeric epitope as disclosed herein such that it enters the subject's system and, thus, is subject to metabolism and other like processes, for example, subcutaneous administration.

The term “optional” or “optionally” means that the subsequent described event, circumstance or substituent may or may not occur, and that the description includes instances where the event or circumstance occurs and instances where it does not.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The term “immunogenic” as used herein means an ability of substance (for example, an antibody, and antibody reagent, or an epitope), such as a chimeric epitope, to elicit an immune response in a host such as a mammal, either humorally or cellularly mediated, or both.

The term “immunogenic composition” used herein is defined as a composition capable of eliciting an immune response, such as an antibody or cellular immune response, or both, when administered to a subject. The immunogenic compositions as disclosed herein may or may not be immunoprotective or therapeutic. When the immunogenic compositions as disclosed herein prevent, ameliorate, palliate or eliminate disease from the subject, then the immunogenic composition may optionally be referred to as a vaccine. As used herein, however, the term immunogenic composition is not intended to be limited to vaccines.

The term “antigen” generally refers to a biological molecule, usually a protein or polypeptide, peptide, polysaccharide, epitope, or conjugate in an immunogenic composition, or immunogenic substance that can stimulate the production of antibodies or T-cell responses, or both, in an animal, including compositions that are injected or absorbed into an animal. The immune response may be generated to the whole molecule (i.e., an HA), or to a various portions of the molecule (e.g., a chimeric epitope). The term may be used to refer to an individual molecule or to a homogeneous or heterogeneous population of antigenic molecules. An antigen is recognized by antibodies, T-cell receptors or other elements of specific humoral and/or cellular immunity. The term “antigen” also includes all related antigenic epitopes. Epitopes of a given antigen can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715; each of which is incorporated herein by reference as if set forth in its entirety. Similarly, conformational epitopes may be identified by determining spatial conformation of amino acids such as by, e.g., x-ray crystallography and 2-dimensional nuclear magnetic resonance. See, e.g., Epitope Mapping Protocols, supra. Furthermore, for purposes of the present invention, “antigen” also can be used to refer to a protein that includes modifications, such as deletions, additions and substitutions (generally conservative in nature, but they may be non-conservative), to the native sequence, so long as the protein maintains the ability to elicit an immunological response. These modifications may be deliberate, as through site-directed mutagenesis, or through particular synthetic procedures, or through a genetic engineering approach, or may be accidental, such as through mutations of hosts, which produce the antigens. Furthermore, the antigen can be derived, obtained, or isolated from a microbe, e.g., a bacterium, or can be a whole organism. Similarly, an oligonucleotide polynucleotide, which expresses an antigen, such as in nucleic acid immunization applications, is also included in the definition. Synthetic antigens are also included, e.g., polyepitopes, flanking epitopes, and other recombinant or synthetically derived antigens (Bergmann et al. (1993) Eur. J. Immunol. 23:2777 2781; Bergmann et al. (1996) J. Immunol. 157:3242-3249; Suhrbier (1997) Immunol. Cell Biol. 75:402 408; Gardner et al. (1998) 12th World AIDS Conference, Geneva, Switzerland, Jun. 28 to Jul. 3, 1998

An “immune response” to chimeric epitope or an immunogenic composition comprising a chimeric epitope is the development in a subject of a humoral and/or a cell-mediated immune response to molecules present in chimeric epitope or an immunogenic composition comprising a chimeric epitope. For purposes of the present invention, a “humoral immune response” is an antibody-mediated immune response and involves the induction and generation of antibodies that recognize and bind with some affinity for the antigen in the immunogenic composition of the invention, while a “cell-mediated immune response” is one mediated by T-cells and/or other white blood cells. A “cell-mediated immune response” is elicited by the presentation of antigenic epitopes in association with Class I or Class II molecules of the major histocompatibility complex (MHC), CD1 or other non-classical MHC-like molecules. This activates antigen-specific CD4+T helper cells or CD8+ cytotoxic lymphocyte cells (“CTLs”). CTLs have specificity for peptide antigens that are presented in association with proteins encoded by classical or non-classical MHCs and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide or other antigens in association with classical or non-classical MHC molecules on their surface. A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to re-stimulation with antigen. Such assays are well known in the art. See, e.g., Erickson et al. (1993) J. Immunol. 151:4189-4199; and Doe et al. (1994) Eur. J. Immunol. 24:2369-2376.

As used herein, the term “broadly neutralizing epitope” refers to an epitope that is within a conserved region of the virus and is not prone to mutation. As such, when the virus mutates to generate a new antigenic strain, the broadly neutralizing epitope is retained. In some embodiments, it is preferred that the broadly neutralizing epitope is shared amongst a plurality of influenza A and/or B subtypes, thus an immune response generated to the broadly neutralizing epitope will confer immunity to at least two different antigenic Influenza A and/or B strains. The term “broadly neutralizing epitope” is also referred to herein as a “conserved donor receptor binding site (RB S),” “neutralizing epitope” or a “donor” portion of a chimeric epitope.

As used herein, the term “molecular scaffold” refers to an influenza virus hemagglutinin that has been “resurfaced” to display one or more epitopes from a different viral strain. Ideally, the molecular scaffold itself does not raise an immune response. In certain embodiments, the epitopes of the influenza virus strain from which the hemagglutinin is derived are removed to prevent a strain-specific immune response. In some embodiments, the molecular scaffold is derived from a non-circulating influenza virus. The molecular scaffold is also referred to herein as the “acceptor” portion of a chimeric epitope.

As used herein, the term “chimeric epitope” refers to an antigenic fusion protein comprising a molecular scaffold and one or more conserved neutralizing epitopes. That is, the chimeric epitope is a recombinant protein comprising polypeptide sequences from two or more proteins (or fragments thereof) which are joined by a peptide bond.

As used herein, the term “fused” means that at least one protein, peptide, or polypeptide is physically associated with a second protein or peptide, such as linkage as a fusion protein.

As used herein, the term “antigenically distinct” refers to a first viral strain that induces a strain-specific host immune response at a different antigenic epitope than that of a second, but related, viral strain. Thus, infection with the first viral strain does not confer immunity to the second viral strain.

The term “site” refers to the location in which the chimeric epitope or an immunogenic composition comprising a chimeric epitope are administered via subcutaneous injection. Examples of potential sites include right deltoid, left deltoid, right and/or vastus lateralis, right and/or subcutaneous tissue on thigh.

The term “dose” refers to a single delivery of chimeric epitope or an immunogenic composition comprising a chimeric epitope to a subject.

The term “mammal” as used herein means a human or non-human animal. More particularly, mammal refers to any animal classified as a mammal, including humans, domestic and farm animals, and zoo, sports and pet companion animals such as a household pet and other domesticated animal including, but not limited to, cattle, sheep, ferrets, swine, horses, rabbits, goats, dogs, cats, and the like. In some embodiments, a companion animal is a dog or cat. Preferably, the mammal is human.

The term “subject” as used herein refers to any animal in which it is useful to elicit an immune response. The subject can be a wild, domestic, commercial or companion animal such as a bird or mammal. The subject can be a human. Although in one embodiment of the invention it is contemplated that the immunogenic compositions as disclosed herein can also be suitable for the therapeutic or preventative treatment in humans, it is also applicable to warm-blooded vertebrates, e.g., mammals, such as non-human primates, (particularly higher primates), sheep, dog, rodent (e.g., mouse or rat), guinea pig, goat, pig, cat, rabbits, cows, and non-mammals such as chickens, ducks, or turkeys. In another embodiment, the subject is a wild animal, for example a bird such as for the diagnosis of avian flu. In some embodiments, the subject is an experimental animal or animal substitute as a disease model. The subject may be a subject in need of veterinary treatment, where eliciting an immune response to an antigen is useful to prevent a disease and/or to control the spread of a disease, for example SIV, STL1, SFV, or in the case of live-stock, hoof and mouth disease, or in the case of birds Marek's disease or avian influenza, and other such diseases.

The term an “immunogenic amount,” and “immunologically effective amount,” both of which are used interchangeably herein, refers to the amount of the chimeric epitope or immunogenic composition sufficient to elicit an immune response, either a cellular (T-cell) or humoral (B-cell or antibody) response, or both, as measured by standard assays known to one skilled in the art.

As used herein, the term “pathogen” refers to an organism or molecule that causes a disease or disorder in a subject. For example, pathogens include but are not limited to viruses, fungi, bacteria, parasites, and other infectious organisms or molecules therefrom, as well as taxonomically related macroscopic organisms within the categories algae, fungi, yeast, protozoa, or the like.

The term “mutant” refers to an organism or cell with any change in its genetic material, in particular a change (i.e., deletion, substitution, addition, or alteration) relative to a wild-type polynucleotide sequence or any change relative to a wild-type protein sequence. The term “variant” may be used interchangeably with “mutant”. Although it is often assumed that a change in the genetic material results in a change of the function of the protein, the terms “mutant” and “variant” refer to a change in the sequence of a wild-type protein regardless of whether that change alters the function of the protein (e.g., increases, decreases, imparts a new function), or whether that change has no effect on the function of the protein (e.g., the mutation or variation is silent).

The term “pharmaceutically acceptable” refers to compounds and compositions which may be administered to mammals without undue toxicity. The term “pharmaceutically acceptable carriers” excludes tissue culture medium. Exemplary pharmaceutically acceptable salts include but are not limited to mineral acid salts such as hydrochlorides, hydrobromides, phosphates, sulfates, and the like, and the salts of organic acids such as acetates, propionates, malonates, benzoates, and the like. Pharmaceutically acceptable carriers are well-known in the art.

The term “variant” as used herein may refer to a polypeptide or nucleic acid that differs from the naturally occurring polypeptide or nucleic acid by one or more amino acid or nucleic acid deletions, additions, substitutions or side-chain modifications, yet retains one or more specific functions or biological activities of the naturally occurring molecule. Amino acid substitutions include alterations in which an amino acid is replaced with a different naturally-occurring or a non-conventional amino acid residue. Such substitutions may be classified as “conservative,” in which case an amino acid residue contained in a polypeptide is replaced with another naturally occurring amino acid of similar character either in relation to polarity, side chain functionality or size. Substitutions encompassed by variants as described herein may also be “non-conservative,” in which an amino acid residue which is present in a peptide is substituted with an amino acid having different properties (e.g., substituting a charged or hydrophobic amino acid with alanine), or alternatively, in which a naturally-occurring amino acid is substituted with a non-conventional amino acid. Also encompassed within the term “variant,” when used with reference to a polynucleotide or polypeptide, are variations in primary, secondary, or tertiary structure, as compared to a reference polynucleotide or polypeptide, respectively (e.g., as compared to a wild-type polynucleotide or polypeptide).

The term “substantially similar,” when used in reference to a variant of an antigen or a functional derivative of an antigen as compared to the original antigen means that a particular subject sequence varies from the sequence of the antigen polypeptide by one or more substitutions, deletions, or additions, but retains at least 50%, or higher, e.g., at least 60%, 70%, 80%, 90% or more, inclusive, of the function of the antigen to elicit an immune response in a subject. In determining polynucleotide sequences, all subject polynucleotide sequences capable of encoding substantially similar amino acid sequences are considered to be substantially similar to a reference polynucleotide sequence, regardless of differences in codon sequence. A nucleotide sequence is “substantially similar” to a given antigen nucleic acid sequence if: (a) the nucleotide sequence hybridizes to the coding regions of the native antigen sequence, or (b) the nucleotide sequence is capable of hybridization to nucleotide sequence of the native antigen under moderately stringent conditions and has biological activity similar to the native antigen protein; or (c) the nucleotide sequences are degenerate as a result of the genetic code relative to the nucleotide sequences defined in (a) or (b). Substantially similar proteins will typically be greater than about 80% similar to the corresponding sequence of the native protein.

Variants can include conservative or non-conservative amino acid changes, as described below. Polynucleotide changes can result in amino acid substitutions, additions, deletions, fusions and truncations in the polypeptide encoded by the reference sequence. Variants can also include insertions, deletions or substitutions of amino acids, including insertions and substitutions of amino acids and other molecules) that do not normally occur in the peptide sequence that is the basis of the variant, for example but not limited to insertion of ornithine which do not normally occur in human proteins. “Conservative amino acid substitutions” result from replacing one amino acid with another that has similar structural and/or chemical properties. Conservative substitution tables providing functionally similar amino acids are well known in the art. For example, the following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W). See, e.g., Creighton, PROTEINS (W.H. Freeman & Co.,1984).

The choice of conservative amino acids may be selected based on the location of the amino acid to be substituted in the peptide, for example if the amino acid is on the exterior of the peptide and exposed to solvents, or on the interior and not exposed to solvents. Selection of such conservative amino acid substitutions is within the skill of one of ordinary skill in the art. Accordingly, one can select conservative amino acid substitutions suitable for amino acids on the exterior of a protein or peptide (i.e. amino acids exposed to a solvent). These substitutions include, but are not limited to the following: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

Alternatively, one can also select conservative amino acid substitutions suitable for amino acids on the interior of a protein or peptide (i.e., the amino acids are not exposed to a solvent). For example, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, LF polypeptides including non-conservative amino acid substitutions are also encompassed within the term “variants.” As used herein, the term “non-conservative” substitution refers to substituting an amino acid residue for a different amino acid residue that has different chemical properties. Non-limiting examples of non-conservative substitutions include aspartic acid (D) being replaced with glycine (G); asparagine (N) being replaced with lysine (K); and alanine (A) being replaced with arginine (R).

The term “derivative” as used herein refers to proteins or peptides which have been chemically modified, for example by ubiquitination, labeling, pegylation (derivatization with polyethylene glycol) or addition of other molecules. A molecule is also a “derivative” of another molecule when it contains additional chemical moieties not normally a part of the molecule. Such moieties can improve the molecule's solubility, absorption, biological half-life, etc. The moieties can alternatively decrease the toxicity of the molecule, or eliminate or attenuate an undesirable side effect of the molecule, etc. Moieties capable of mediating such effects are disclosed in REMINGTON'S PHARMACEUTICAL SCIENCES (21st ed., Tory, ed., Lippincott Williams & Wilkins, Baltimore, Md., 2006).

The term “functional” when used in conjunction with “derivative” or “variant” refers to a protein molecule which possesses a biological activity that is substantially similar to a biological activity of the entity or molecule of which it is a derivative or variant. “Substantially similar” in this context means that the biological activity, e.g., antigenicity of a polypeptide, is at least 50% as active as a reference, e.g., a corresponding wild-type polypeptide, e.g., at least 60% as active, 70% as active, 80% as active, 90% as active, 95% as active, 100% as active or even higher (i.e., the variant or derivative has greater activity than the wild-type), e.g., 110% as active, 120% as active, or more, inclusive.

The terms “significantly different than”, “statistically significant,” and similar phrases refer to comparisons between data or other measurements, wherein the differences between two compared individuals or groups are evidently or reasonably different to the trained observer, or statistically significant (if the phrase includes the term “statistically” or if there is some indication of statistical test, such as a p-value, or if the data, when analyzed, produce a statistical difference by standard statistical tests known in the art).

The term “adjuvant” as used herein refers to any agent or entity which increases the antigenic response or immune response by a cell or organism to a target antigen. Examples of adjuvants include, but are not limited to, mineral gels such as aluminum hydroxide or aluminum phosphate; surface active substances such as lysolecithin, pluronic polyols, polyanions; other peptides; oil emulsions; and potentially useful human adjuvants such as BCG and Corynebacterium parvum, QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, Albumin (Alum), CpG ODN, Betafectin, and MF59. In one embodiment, the adjuvant is not Freund's adjuvant (particularly for immunization of human subjects). In another embodiment, the adjuvant is a mucosal adjuvant (e.g., multiply mutated cholera toxin (mmCT).

As used herein, “prevention” or “preventing,” when used in reference to a disease, disorder or symptoms thereof, refers to a reduction in the likelihood that an individual will develop a disease or disorder, e.g., cholera, as but one example. The likelihood of developing a disease or disorder is reduced, for example, when an individual having one or more risk factors for a disease or disorder either fails to develop the disorder or develops such disease or disorder at a later time or with less severity, statistically speaking, relative to a population having the same risk factors and not receiving treatment as described herein. The failure to develop symptoms of a disease, or the development of reduced (e.g., by at least 10% on a clinically accepted scale for that disease or disorder) or delayed (e.g., by days, weeks, months or years) symptoms is considered effective prevention.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase” or “enhance” or “activate” are all used herein to generally mean an increase of a property, level, or other parameter by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Although the foregoing invention has been described in some detail by way of illustration and example for purposes of clarity of understanding it will be readily apparent to one of ordinary skill in the art in light of the teachings of this invention that certain changes and modifications may be made thereto without departing from the spirit or scope of the appended claims. The following is meant to be illustrative of the present invention; however, the practice of the invention is not limited or restricted in any way by the examples.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### BRIEF DESCRIPTION OF THE DRAWINGS

FIGS. 1A-1E show RBS grafts and sequence alignments. (FIG. 1A) Phylogeny of influenza subtypes. Group 1 and 2 influenzas are annotated. (FIG. 1B) Representative H1 antigenic clusters: H1 Massachusetts/1/1990 (MA-90), H1 Solomon Islands/03/2006 (SI-06) and H1 California/07/2009 (CA-09) are listed. Sequence alignment is in reference to SI-06 and conserved residues are marked as (.); segments defining the H1 RBS graft, S1-4, are colored. (FIG. 1C) Residues comprising S1-4 of the acceptor scaffolds from non-circulating influenzas H4 New Brunswick/00464/2010 (H4 NB-10), H6 Wisconsin/617/1983 (H6 WI-83), H14 Wisconsin/10OS3941/2006 (H14 WI-06) and H16 Delaware Bay/296/1998 (H16 DB-98). (FIG. 1D) Influenza HA trimer (PDB 5UGY) in surface representation. HA1 is in silver, HA2 in dark gray and S1-4 are colored. (FIG. 1E) LSTc (stick representation) modeled in complex with HA. S1-4 is colored and HA is in silver.

FIGS. 2A-2E show the structure of K03.12 in complex with rsH4NBv1 and scaffold improvement. (FIG. 2A) Antibody K03.12 Fab (heavy and light chains are colored blue and green, respectively) in complex with rsH4NBv1 HA1 “head” (silver). The CDR H3 (magenta) is marked. (FIG. 2B) Close-up of the antigen combining site. The CDR H3 (magenta) is shown in sticks with key interacting HA residues. Hydrogen bonds are denoted in yellow, dashed-lines. (FIG. 2C) Comparison of the RBS of H1 SI-06, yellow, (PDB 4YJZ), H4 A/duck/Czechoslovakia/1956 (H4 CZ-56), black (PDB 5XL3) and rsH4NBv1, silver. The segments of the grafts are labeled and colored. (FIG. 2D) rsH4NBv2 and (FIG. 2E) rsH4NBv3 with residues changed for each construct represented by a colored sphere. The original, unchanged segments from rsH4NBv1 are labeled and colored.

FIGS. 3A-3J present data showing reactivity of RBS-directed IgGs for rsHAs. CH67 (blue), H2526 (red), H2227 (green), 641 1-9 (violet) and K03.12 (orange) IgGs were titrated against (FIG. 3A) wildtype H4 NB-10, (FIG. 3B) rsH4NBv1, (FIG. 3C) rsH4NBv1, (FIG. 3D) rsH4NBv3, (FIG. 3E) KDs obtained from curves in FIGS. 3A-3D for H4 constructs. IgG titrations for (FIG. 3F) wildtype H14 WI-10, (FIG. 3G) rsH14v1, (FIG. 3H) rsH14v2 and (FIG. 3I) control H1 SI-06 HA constructs. (FIG. 3J) KDs obtained from curves in F-I for H14 constructs. ELISA measurements were done in duplicate over the concentration range except for FIG. 3A and FIG. 3E where only 1 μM final concentration of IgG was tested. The wildtype H1 SI-06 values in FIG. 3E and FIG. 3J are both derived from FIG. 3I. Curve fitting and KDs were obtained using GraphPad Prism version 6.0.

FIGS. 4A-4C show conservation of the H1 HA RBS and critical SA contacts. (FIG. 4A) Sequence alignment of historical H1 RBS and critical residues comprising sialic acid (SA) contacts. The segments in the RBS graft are colored. Conserved residues (.) are in reference to H1 SI-06. The representative antigenic clusters (C1, C2, C3) of H1 isolates are listed with the numbering of circulating corresponding years (y). In order, the representative H1 isolates are: USSR-1977, Taiwan-1980, Singapore-1986, Massachusetts-1990, Wellington-1990, Florida-1993, Bejing-1999, Florida-1990, North Carolina-2003, Solomon Islands-2006, Brisbane-2007, North Carolina-2008, California-2009, Wisconsin-2010, Texas-2010, Mexico-2012, Hawai'i-2015, New York-2017. (FIG. 4B) SA-contact residues in the RBS-graft segments are noted as asterisks (magenta) with the additional, conserved SA-contacts not present in the graft are listed. (FIG. 4C) Exemplary acceptor scaffold sequences.

FIGS. 5A-5D show conservation of HAs. Using H1 SI-06 as reference, residue conversation is shown in red for historical H1 MA-90 and the new pandemic, H1 California/04/2009 (H1 CA-09) as well as the two acceptor scaffolds H4 NB-10 and H14 WI-10. Two views are shown: top is the HA trimer in spacefill with only one monomer colored red at points of conservation; the bottom is a close-up the RBS with a LStc molecule (stick-representation) docked for point of reference. The percent identity is in reference to H1-SI-06 with the total number of insertions and deletions listed. For both H1 MA-90 and H1 CA-09, the insertion is residue K133a.

FIGS. 6A-6D show biochemical characterization of the optimized rsHAs. (FIG. 6A) Coomassie-stained SDS-PAGE gel of the rsH4NBv3 head and FLsE constructs (marked “1” and “2”, respectively). The doublet for the FLsE construct is proteolysis of the purification tags. A prestained protein ladder is in the first and the corresponding molecular weights (in kilodaltons) are marked. (FIG. 6B) Representative FPLC trace using a Superdex 10/300 column of the FLsE construct. The trace monitors the absorbance (in mAU) at 280 nm as a function of elution volume (mL). (FIG. 6C) Coomassie-stained SDS-PAGE of the rsH14WIv2 constructs (labeled as in A)) and (FIG. 6D) representative FPLC trace (similar to C)).

FIGS. 7A-7C show antibody footprints of RBS-directed antibodies. RBS-directed antibodies used in this study to obtain ELISA and BLI binding affinities. (FIG. 7A) Crystal structures of CH67 (PDB 4HKX), 641 1-9 (PDB 4YK4), H2526 (PDB 4YJZ), K03.12 Fab (PDB 5W08) and C05 (PDB 4FQR) in complex with HA (silver). The variable heavy and light domains are colored blue and green, respectively with the CDR H3 in the antigen combining site shown in magenta. (FIG. 7B) An approximate angle of approach of the CDR H3 of each antibody with the HA RBS. (FIG. 7C) The overall footprint of each antibody with HA is shown in cyan. All figures were created using PyMol.

FIGS. 8A-8D show K03.12 Fab structure. (FIG. 8A) K03.12 Fab (heavy and light chains are colored blue and green, respectively) in complex with rsH4NBy 1 HA1 “head” (silver). The CDR H3 (magenta) is marked. (FIG. 8B) K03.12 Fab (heavy and light chains are colored blue and green, respectively) in complex with H3 TX-12 (PDB 5W08) HA1 “head” (silver). The CDR H3 (magenta) is marked. (FIGS. 8C and 8D) Close-up of the antigen combining site. The CDR H3 (magenta) is shown in sticks with key interacting HA residues (silver). Hydrogen bonds are denoted in yellow, dashed-lines.

FIG. 9 presents experimental data showing immunogenicity of an initial resurfaced HA (rsHA) scaffold H4NBv3 (rsH4). These data are from day 8 (d8) primary responses in human J_{H}6 mice. rsH4 is as immunogenic as control wildtype influenza HA derived from H1 Solomon Islands/03/2006 (H1 SI-06) as measured by plasma blasts/cytes and GC B cells induction (lower panels).

FIGS. 10A-10C Preliminary analyses of the serum and single GC B-cell responses are shown in FIG. 10A. The data show that the heterologous boost was as efficient as the homologous boost at eliciting serum responses that directly competed with the RBS-directed bnAb CH67. This indicates that rsHA immunogen, which only has the H1 RBS epitope in common with the initial priming immunogen, can effectively recall memory responses to the H1 RBS. Single GC B-cell analysis indicates that the homologous prime-boost produces very few B cells that interact with the rsH4NB10v3 and none that bind the wildtype H4 scaffold (FIG. 10B). In contrast, the heterologous boost with rsHA produces robust responses to the rsH4NB10v3 immunogen. H4 scaffold-specific responses were also observed. FIG. 10C, quantification of the total number of CH67-like single GC B-cells and preliminary data indicate an overall increase of such cells resulting from the heterologous boost with rsH4NBv3.

FIGS. 11A-11B Structure and structure-based modifications. FIG. 11A, K03.12 Fab incomplex with rsH4 with H1 S1-06 RBS graft. FIGS. 11B, HC19 Fab docked into the rsH4 with the H3 AI-68 RBS graft.

FIGS. 12A-12C Design of Cys-Cys trimeric heads. FIG. 12A, FlsE HA trimer and the trimeric HA head. SDS-PAGE analysis of FIG. 12B wt H14 WI-10. FIG. 12C, rsH14 WI-10^{(H3)} under non-reducing (N.R.) and reducing (R.) conditions.

FIG. 13 Exemplary workflow for B-cell repertoire analysis.

FIGS. 14A-14C Immunization strategy. FIG. 14A, homotrimeric rsHAcys-cys prime-boost. FIG. 14B, wtHA FlsE prime with homotrimeric rsHAcys-cys boost. FIGS. 14C, as in FIG. 14B but with heterotrimeric rsHAcys-cys boost. Day of sample isolation method.

FIG. 15 Schematic of constructs. HA: hemagglutinin; FlsE: full length soluble ectodomain; rsHA: resurfaced HA. Arrows show progression of design to an optimized immunogen(s).

FIG. 16 Exemplary sequences for CDR3 of select bnAbs that target the RBS.

### DETAILED DESCRIPTION OF THE INVENTION

Influenza viruses are constantly evolving such that viruses that are antigenically different than the original virus (i.e., “antigenic drift”) are produced. When antigenic drift occurs, a host's immune system may not recognize the newer virus. Thus, production of an effective annual flu vaccine requires that the scientific community accurately predict the strain or strains of influenza that are expected to be prevalent in a given year. Unfortunately, the production process itself takes considerable time and there is not a good way to quickly modify a vaccine for another strain. These issues with effective vaccination can be avoided if a vaccine can be generated that targets one or more conserved regions of a family of influenza viruses, thus producing broadly neutralizing antibodies in a host that can target multiple different viral strains. Provided herein is a method for generating a vaccine that produces broadly neutralizing antibodies in a host. Although the description throughout refers specifically to influenza viruses, it is specifically contemplated herein that any virus that undergoes rapid antigenic drift can benefit from the methods described herein.

The methods and compositions provided herein are based, in part, on the discovery that grafting conserved receptor binding sites of circulating influenza viruses (e.g., H1 influenza viruses) to existing hemagglutinin sites in non-circulating influenza viruses can result in the production of broadly neutralizing antibodies in a host.

### Influenza

Influenza is an infectious illness caused by an influenza virus and is characterized by a high fever, runny nose, sore throat, muscle pains, headache, coughing and fatigue. Such symptoms can range widely in severity and can be worse in young children, immunocompromised individuals or in the elderly.

Influenza outbreaks occur on an annual basis and the illness can spread among individuals through airborne or surface transmission of the virus. Thus, rigorous hand-washing hygiene is a preferred method for reducing the risk of viral spread.

Current treatment for an active flu infection is limited and includes administration of anti-viral treatments (e.g., aseltamivir, amantadine/rimandadine), and over-the-counter medications for pain and symptom management. Treatment with antivirals is sub-optimal in that treatment needs to be initiated within 48 hours of symptom onset.

Vaccination plays an important role in controlling influenza epidemics. Currently available influenza vaccines are generally either inactivated or live attenuated influenza vaccines. Inactivated flu vaccines are composed of three possible forms of antigen preparation: inactivated whole virus, sub-virions where purified virus particles are disrupted with detergents or other reagents to solubilize the lipid envelope (so-called “split” vaccine) or purified HA and NA (subunit vaccine). These inactivated vaccines are usually given intramuscularly (i.m.), subcutaneously (s.c), or intranasally (i.n.).

Influenza vaccines for interpandemic use (also termed seasonal), of all kinds, are usually trivalent vaccines. They generally contain antigens derived from two influenza A-type virus strains and one influenza B-type virus strain. A standard 0.5 ml injectable dose in most cases contains (at least) 15 μg of HA from each strain, as measured by single radial immunodiffusion (SRD) (J. M. Wood et al.: An improved single radial immunodiffusion technique for the assay of influenza hemagglutinin antigen: adaptation for potency determination of inactivated whole virus and subunit vaccines. J. Biol. Stand. 5 (1977) 237-247; J. M. Wood et al., International collaborative study of single radial diffusion and immunoelectrophoresis techniques for the assay of hemagglutinin antigen of influenza virus. J. Biol. Stand. 9 (1981) 317-330). Usually, those vaccines are unadjuvanted.

Although such influenza vaccines are available yearly, they are not always effective because the selection of strains for the influenza vaccine composition does not match the strains that are dominant during a give flu season. In addition, influenza viruses are prone to antigenic drift and antigenic shift, thus making it difficult to generate a single vaccination for multiple strains or for long-term immunity.

Currently, only influenza type A H1 and H3 and type B viruses are circulating in humans. Thus, these influenza viruses are the only ones included in the seasonal vaccine.

### Influenza Virus and Viral Strains

Influenza viruses are one of the most ubiquitous viruses present in the world, affecting both humans and livestock. Influenza results in significant economic burden, morbidity and even mortality. There are three types of influenza viruses that infect humans: A, B and C. Generally, human influenza viruses A and B are responsible for seasonal epidemics of the flu. Influenza type C infections typically cause a mild respiratory illness and are not thought to cause epidemics at this time.

The influenza virus is an enveloped virus which comprises an internal nucleocapsid or core of ribonucleic acid (RNA) associated with nucleoprotein, surrounded by a viral envelope with a lipid bilayer structure and external glycoproteins. The inner layer of the viral envelope is composed predominantly of matrix proteins and the outer layer mostly of host-derived lipid material. Influenza virus comprises two surface antigens, glycoproteins neuraminidase (NA) and hemagglutinin (HA), which appear as spikes at the surface of the particles. It is these surface proteins, particularly HA that determine the antigenic specificity of the influenza subtypes.

Virus strains are classified according to host species of origin, geographic site and year of isolation, serial number, and, for influenza A, by serological properties of subtypes of hemagglutinin (HA) and neuraminidase (NA). Influenza A virus currently displays eighteen HA subtypes: H1, H2, H3, H4, H5, H6, H7, H8, H9, H10, H11, H12, H13, H14, H15, H16, H17 and H18, as well as nine NA subtypes N1, N2, N3, N4, N5, N6, N7, N8 or N9. Viruses of all HA and NA subtypes have been recovered from aquatic birds, but only three HA subtypes (H1, H2, and H3) and two NA subtypes (N1 and N2) have established stable lineages in the human population since 1918. Specific examples of influenza subtypes that have been confirmed to cause illness in humans include H1N1 (“Spanish flu”), H2N2 (“Asian flu”), H3N2 (“Hong Kong flu”), H5N1, H7N7, H1N2, H9N2, H7N2, H7N3, H5N2, and H1ON7.

Only one subtype of HA and one of NA are recognized for influenza B viruses. Current influenza B viral strains are either BNictoria/2/87-like or B/Yamagata/16/88-like. These strains are usually distinguished antigenically, but differences in amino acid sequences have also been described for distinguishing the two lineages e.g. B/Yamagata/16/88-like strains often (but not always) have HA proteins with deletions at amino acid residue 164, numbered relative to the ‘Lee40’ HA sequence (GenBank sequence ID: GI325176).

It is preferred that the influenza vaccine as described herein comprises an epitope within a well conserved region of the influenza A and influenza B subtypes. That is, it is preferred that the epitope is shared amongst a number of viruses to permit immunity to multiple strains and also long-term immunity. One of skill in the art can easily determine conserved regions among the Influenza A and B type viruses by performing a sequence alignment and identifying conserved regions. Such methods are well within the abilities of one of skill in the art and as such are not described herein.

Exemplary H1 sequences comprising the RBS site that can be used in the generation of chimeric antibodies as described herein include, but are not limited to the following sequences.

SEQ ID NOs 1-12, shown below in order of appearence, are the amino acid sequences of historical H1 isolates. In SEQ ID NOs 1-12, the bolded text represents the conserved segments that comprise the receptor binding site (RBS).

| | |
|---|---|
| A/USSR/90/1977 | APLQLGKCNIAGWILGNPECESLFSKKSWSYIAETPNSENGTCYPGYFAD |
| A/Kawasaki/6/1986 | APLQLGNCSIAGWILGNPECESLFSKKSWSYIAETPNSENGTCYPGYFAD |
| A/Massachusetts/1/1990 | APLQLGNCSIAGWILGNPECESLFSKESWSYIAETPNSENGTCYPGYFAD |
| A/Wellington/47/1992 | APLQLGNCSIAGWILGNPECESLFSKESWSYIAETPDPENGTCYPGYFAD |
| A/Florida/2/1993 | APLQLGNCSVAGWILGNPECESLFTKESWSYIAETPNPENGTCYPGYFAD |
| A/Beijing/262/1995 | APLQLGNCSVAGWILGNPECESLISKESWSYIVETPNPENGTCYPGYFAD |
| A/New Caledonia/20/1999 | APLQLGNCSVAGWILGNPECELLISKESWSYIVETPNPENGTCYPGYFAD |
| A/North Carolina/2003 | APLQLGNCSVAGWILGNPECELLISKESWSYIVETPNPENGTCYPGYFAD |
| A/North Carolina/2007 | APLQLGNCSVAGWILGNPECELLISKESWSYIVETPNPENGTCYPGYFAD |
| A/Solomon Islands/03/2006 | APLQLGNCSVAGWILGNPECELLISRESWSYIVEKPNPENGTCYPGHFAD |
| A/North Carolina/2008 | APLQLGNCSVAGWILGNPECELLISKESWSYIVEKPNPENGTCYPGHFAD |
| A/California/2009 | APLHLGKCNIAGWILGNPECESLSTASSWSYIVETPSSDNGTCYPGDFID |
| | *** ** * *********** * ***** * * ******* * * |
| | 133a |
| A/USSR/90/1977 | YEELREQLSSVSSFERFEIFPKERSWPKHNVTRGVTASCSHKGKSSFYRN |
| A/Kawasaki/6/1986 | YEELREQLSSVSSFERFEIFPKESSWPNHTVTKGVTASCSHKGRSSFYRN |
| A/Massachusetts/1/1990 | YEELREQLSSVSSFERFEIFPKESSWPNHTVTKGVTASCSHNGKSSFYRN |
| A/Wellington/47/1992 | YEELREQLSSVSSFERFEIFPKESSWPNHTVTKGVTASCSHNGKSSFYRN |
| A/Florida/2/1993 | YEELREQLSSVSSFERFEIFPKESSWPNHTVTKGVTASCSHNGKSSFYRN |
| A/Beijing/262/1995 | YEELREQLSSVSSFERFEIFPKESSWPNHTVT-GVTASCSHNGKSSFYRN |
| A/New Caledonia/20/1999 | YEELREQLSSVSSFERFEIFPKESSWPTHTVT-GVSASCSHNGKSSFYRN |
| A/North Carolina/2003 | YEELREQLSSVSSFERFEIFPKESSWPHHTVT-GVSASCSHNGKSSFYRN |
| A/North Carolina/2007 | YEELREQLSSVSSFERFEIFPKESSWPNHTVT-GVSASCSHNGKSSFYRN |
| A/Solomon Islands/03/2006 | YEELREQLSSVSSFERFEIFPKESSWPNHTTT-GVSASCSHNGESSFYKN |
| A/North Carolina/2008 | YEELREQLSSVSSFERFEIFPKESSWPNHTVT-GVSASCSHNGESSFYRN |
| A/California/2009 | YEELREQLSSVSSFERFEIFPKTSSWPNHDSNKGVTAACPHAGAKSFYKN |
| | ********************** *** * * * * * * *** * |
| | 190-helix |
| A/USSR/90/1977 | LLWLTEKNGSYPNLSKSYVNNKEKEVLVLWGVHHPSNIEDQKTIYRKENA |
| A/Kawasaki/6/1986 | LLWLTEKNGSYPNLSKSYVNNKEKEVLVLWGVHHPSNIRDQRAIYHTENA |
| A/Massachusetts/1/1990 | LLWLTEKNGLYPNLSKSYVNNKEKEVLVLWGVHHPSNIGDQRAIYHTENA |
| A/Wellington/47/1992 | LLWLTKKNGLYPNLSKSYVNNKEKEVLVLWGVHHPSNIGDQRAIYHTENA |
| A/Florida/2/1993 | LLWLTEKNGLYPNLSKSYVNNKEKEVLVLWGVHHPSNMGDQRAIYHTENA |
| A/Beijing/262/1995 | LLWLTEKNGLYPNLSNSYVNNKEKEVLVLWGVHHPSNIRDQRAIYHTENA |
| A/New Caledonia/20/1999 | LLWLTGKNGLYPNLSKSYVNNKEKEVLVLWGVHHPPNIGDQRALYHTENA |
| A/North Carolina/2003 | LLWLTGKNGLYPNLSKSYTNNKEKEVLVLWGVHHPPNIGDQRALYHTENA |
| A/North Carolina/2007 | LLWLTGKNGLYPNLSKSYANNKEKEVLVLWGVHHPPNIGDQRALYHTENA |
| A/Solomon Islands/03/2006 | LLWLTGKNGLYPNLSKSYANNKEKEVLVLWGVHHPPNIGDQRALYHTENA |
| A/North Carolina/2008 | LLWLTGKNGLYPNLSKSYANNKEKEVLVLWGVHHPPNIGDQKALYHTENA |
| A/California/2009 | LIWLVKKGNSYPKLSKSYINDKGKEVLVLWGIHHPSTSADQQSLYQNADA |
| | * ** * ** ** ** * * ******** *** ** * * |
| A/USSR/90/1977 | YVSVVSSNYNRRFTPEIAERPKVRGQAGRINYYWTLLEPGDTIIFEANGN |
| A/Kawasaki/6/1986 | YVSVVSSHYNRRFTPEIAKRPKVRDQEGRINYYWTLLEPGDTIIFEANGN |
| A/Massachusetts/1/1990 | YVSVVSSHYSRRFTPEIAKRPKVRDQEGRINYYWTLLEPGDTIIFEANGN |
| A/Wellington/47/1992 | YVSVVSSHYSRRFTPEIAKRPKVRDQEGRINYYWTLLEPGDTIIFEANGN |
| A/Florida/2/1993 | YVSVVSSHYSRRFTPEIAKRPKVRDQEGRINYYWTLLEPGDTIIFEANGN |
| A/Beijing/262/1995 | YVSVVSSHYSRRFTPEIAKRPKVRGQEGRINYYWTLLEPGDTIIFEANGN |
| A/New Caledonia/20/1999 | YVSVVSSHYSRRFTPEIAKRPKVRDQEGRINYYWTLLEPGDTIIFEANGN |
| A/North Carolina/2003 | YVSVVSSHYSRRFTPEIAKRPKVRDQEGRINYYWTLLEPGDTIIFEANGN |
| A/North Carolina/2007 | YVSVVSSHYSRRFTPEIAKRPKVRDQEGRINYYWTLLEPGDTIIFEANGN |
| A/Solomon Islands/03/2006 | YVSVVSSHYSRKFTPEIAKRPKVRDQEGRINYYWTLLEPGDTIIFEANGN |
| A/North Carolina/2008 | YVSVVSSHYSRKFTPEIAKRPKVRDQEGRINYYWTLLEPGDTIIFEANGN |
| A/California/2009 | YVFVGSSRYSKKFKPEIAIRPKVRDQEGRMNYYWTLVEPGDKITFEATGN |
| | ** * ** * * **** ***** * ** ****** **** * *** ** |
| A/USSR/90/1977 | LIAPWHAFALNRGFGSG |
| A/Kawasaki/6/1986 | LIAPWYAFALSRGFGSG |
| A/Massachusetts/1/1990 | LIAPWYAFALSRGLGSG |
| A/Wellington/47/1992 | LIAPWYAFALSRGFGSG |
| A/Florida/2/1993 | LIAPRYAFALSRGFGSG |
| A/Beijing/262/1995 | LIAPWYAFALSRGFGSG |
| A/New Caledonia/20/1999 | LIAPWYAFALSRGFGSG |
| A/North Carolina/2003 | LIAPRYAFALSRGFGSG |
| A/North Carolina/2007 | LIAPRFAFALSRGFGSG |
| A/Solomon Islands/03/2006 | LIAPRYAFALSRGFGSG |
| A/North Carolina/2008 | LIAPRYAFALSRGFGSG |
| A/California/2009 | LVVPRYAFAMERNAGSG |
| | * * *** * *** |

SEQ ID NOs 13-23, shown below in order of appearence, are the amino acid sequences of historical H3 isolates. In SEQ ID NOs 13-23, the bolded text represents the conserved segments that comprise the receptor binding site (RBS).

Historical H3 isolates; bolded text are the conserved segments that comprise the receptor
binding site (RBS).

| | | |
|---|---|---|
| X-31 (68) | TNATELVQSSSTGKICNNPHRILDGIDCTLIDALLGDPHCDVFQNETWDLFVERSKAFSN | 60 |
| Bangkok (79) | TNATELVQSSSTGRICDSPHRILDGKNCTLIDALLGDPHCDGFQNEKWDLFVERSKAFSN | 60 |
| Philippines (82) | TNATELVQSSSTGRICDSPHRILDGKNCTLIDALLGDPHCDGFQNEKWDLFVERSKAFSN | 60 |
| Leningrad (86) | TNATELVQSSSTGRICDSPHRILDGKNCTLIDALLGDPHCDGFQNEKWDLFIERSKAFSN | 60 |
| Beijing (89) | TNATELVQSSSTGRICDSPHRILDGKNCTLIDALLGDPHCDGFQNKEWDLFVERSKAYSN | 60 |
| Joburg (94) | TNATELVQSSPTGRICDSPHRILDGKNCTLIDALLGDPHCDGFQNKEWDLFVERSKAYSN | 60 |
| WI (05) | TNATELVQSSSTGGICDSPHQILDGENCTLIDALLGDPQCDGFQNKKWDLFVERSKAYSN | 60 |
| Moscow (09) | TNATELVQSSSTGRICDSPHQILDGENCTLIDALLGDPHCDGFQNKEWDLFVERSKAYSN | 60 |
| Victoria (11) | TNATELVQNSSIGEICDSPHQILDGENCTLIDALLGDPQCDGFQNKKWDLFVERSKAYSN | 60 |
| Texas (12) | TNATELVQNSSIGEICDSPHQILDGENCTLIDALLGDPQCDGFQNKKWDLFVERSKAYSN | 60 |
| HK (14) | TNATELVQNSSIGEICDSPHQILDGENCTLIDALLGDPQCDGFQNKKWDLFVERSKAYSN | |
| | ********.* * **:.**:**** :***********:** ***: ****:*****:** | |
| X-31 (68) | CYPYDVPDYASLRSLVASSGTLEFITEGFTWTGVTQNGGSNACKRGPGSGFFSRLNWLTK | 120 |
| Bangkok (79) | CYPYDVPDYASLRSLVASSGTLEFINEGFNWTGVTQSGGSYACKRGSDNSFFSRLNWLYE | 120 |
| Philippines (82) | CYPYDVPDYASLRSLVASSGTLEFINEGFNWTGVTQSGGSSTCKRGSNNSFFSRLNWLYE | 120 |
| Leningrad (86) | CYPYDVPDYASLRSLVASSGTLEFINEGFNWTGVTQSGGSYTCKRGSVNSFFSRLNWLYE | 120 |
| Beijing (89) | CYPYDVPDYASLRSLVASSGTLEFINEDFNWTGVAQSGESYACKRGSVKSFFSRLNWLHE | 120 |
| Joburg (94) | CYPYDVPDYASLRSLVASSGTLEFINENFNWTGVAQDGKSYACKRGSVNSFFSRLNWLHK | 120 |
| WI (05) | CYPYDVPDYASLRSLVASSGTLEFNDESFNWTGVTQNGTSSSCKRRSNNSFFSRLNWLTQ | 120 |
| Moscow (09) | CYPYDVPDYASLRSLVASSGTLEFNNESFNWTGVAQNGTSSACKRRSIKSFFSRLNWLHQ | 120 |
| Victoria (11) | CYPYDVPDYASLRSLVASSGTLEFNNESFNWTGVTQNGTSSACIRRSNNSFFSRLNWLTR | 120 |
| Texas (12) | CYPYDVPDYASLRSLVASSGTLEFNNESFNWNGVTQNGTSSACIRRSNNSFFSRLNWLTH | 120 |
| HK (14) | CYPYDVPDYASLRSLVASSGTLEFNNESFNWTGVTQNGTSSACIRRSSSSFFSRLNWLTH | |
| | ************************ * *.*.**:*.* * :* * ..******** | |
| X-31 (68) | SGSTYPVLNVTMPNNDNFDKLYIWGIHHPSTDQEQTSLYVQASGRVTVSTRRSQQTIIPN | 180 |
| Bangkok (79) | SESKYPVLNVTMPNNGNFDKLYIWGVHHPSTDKEQTKLYVRASGRVTVSTKRSQQTIIPN | 180 |
| Philippines (82) | SESKYPVLNVTMPNNGKFDKLYIWGIHHPSTDKDQTNLYIRASGRVTVSTKRSQQTVIPN | 180 |
| Leningrad (86) | SEYKYPALNVTMPNNGKFDKLYIWGVHHPSTEKEQTNLYVRASGRVTVSTKRSQQTVIPN | 180 |
| Beijing (89) | SEYKYPALNVTMPNNGKFDKLYIWGVHHPSTDREQTKLYVRASGRVTVSTKRSQQTVIPN | 180 |
| Joburg (94) | LEYKYPALNVTMPNNGKFDKLYIWGVHHPSTDSDQTSLYVRASGRVTVSTKRSQQTVIPD | 180 |
| WI (05) | LKFKYPALNVTMPNNEKFDKLYIWGVHHPVTDNDQIFLYAQASGRITVSTKRSQQTVIPN | 180 |
| Moscow (09) | LENRYPALNVTMPNNDKFDKLYIWGVHHPSTDSVQTSVYVQASGRVTVSTKRSQQTVIPN | 180 |
| Victoria (11) | LNFKYPALNVTMPNNEQFDKLYIWGVHHPVTDKEQIFLYAQSSGRITVSTKRSQQAVIPN | 180 |
| Texas (12) | LNFKYPALNVTMPNNEQFDKLYIWGVHHPVTDKDQIFLYAQPSGRITVSTKRSQQAVIPN | 180 |
| HK (14) | LNYTYPALNVTMPNKEQFDKLYIWGVHHPGTDKDQIFLYAQSSGRITVSTKRSQQAVIPN | |
| | **.******** :********:*** . * :* : ***:****:****::**: | |
| X-31 (68) | IGSRPWVRGLSSRISIYWTIVKPGDVLVINSNGNLIAPRGYFKMRTGKSSIMRSDAPIDT | 240 |
| Bangkok (79) | IGSRPWVRGLSSGISIYWTIVKPGDILLINSNGNLIAPRGYFKIRTGKSSIMRSDAPIGT | 240 |
| Philippines (82) | IGSRPWVRGLSSRISIYWTIVKPGDILLINSIGNLIAPRGYFKIRTGKSSIMRSDAPIGT | 240 |
| Leningrad (86) | IGSSPWVRGLSSRISIYWTIVKPGDILLINSTGNLIAPRGYFKIRTGKSSIMRSDAPIGT | 240 |
| Beijing (89) | IGSRPWVRGLSSRISIYWTIVKPGDILLINSTGNLIAPRGYFKIRTGKSSIMRSDAPIGT | 240 |
| Joburg (94) | IGYRPWVRGQSSRISIYWTIVKPGDILLINSTGNLIAPRGYFKIRNGKSSIMRSDAPIGN | 240 |
| WI (05) | IGSRPRIRNIPSRISIYWTIVKPGDILLINSTGNLIAPRGYFKIRSGKSSIMRSDAPIGK | 240 |
| Moscow (09) | IGSRPWVRGVSSRISIYWTIVKPGDILLINSTGNLIAPRGYFKIRSGKSSIMRSDAPIGK | 240 |
| Victoria (11) | IGYRPRIRNIPSRISIYWTIVKPGDILLINSTGNLIAPRGYFKIRSGKSSIMRSDAPIGK | 240 |
| Texas (12) | IGFRPRIRNIPSRISIYWTIVKPGDILLINSTGNLIAPRGYFKIRSGKSSIMRSDAPIGK | 240 |
| HK (14) | IGSRPRIRDIPSRISIYWTIVKPGDILLINSTGNLIAPRGYFKIRSGKSSIMRSDAPIGK | |
| | ** * :* . ************:*:*** ***********:*.************ . | |
| X-31 (68) | CISECITPNGSIPNDKPFQNVNKITYGACPKYVKQNTLKVGTG | 283 |
| Bangkok (79) | CSSECITPNGSIPNDKPFQNVNKITYGACPKYVKQNTLKLATG | 283 |
| Philippines (82) | CSSECITPNGSIPNDKPFQNVNKITYGACPRYVKQNTLKLATG | 283 |
| Leningrad (86) | CSSECITPNGSIPNDKPFQNVNKITYGACPRYVKQNTLKLATG | 283 |
| Beijing (89) | CSSECITPNGSIPNDKPFQNVNRITYGACPRYVKQNTLKLATG | 283 |
| Joburg (94) | CSSECITPNGSIPNDKPFQNVNRITYGACPRYVKQNTLKLATG | 283 |
| WI (05) | CNSECITPNGSIPNDKPFQNVNRITYGACPRYVKQNTLKLATG | 283 |
| Moscow (09) | CNSECITPNGSIPNDKPFQNVNRITYGACPRYVKQNTLKLATG | 283 |
| Victoria (11) | CNSECITPNGSIPNDKPFQNVNRITYGACPRYVKQSTLKLATG | 283 |
| Texas (12) | CKSECITPNGSIPNDKPFQNVNRITYGACPRYVKQSTLKLATG | |
| HK (14) | CKSECITPNGSIPNDKPFQNVNRITYGACPRYVKHSTLKLATG | |
| | * ********************:*******:****.***:.** | |

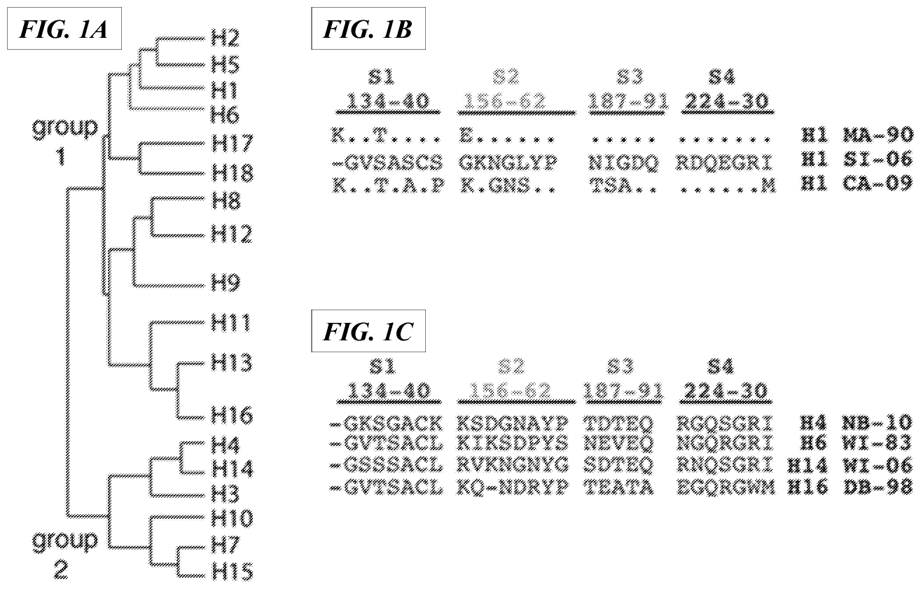

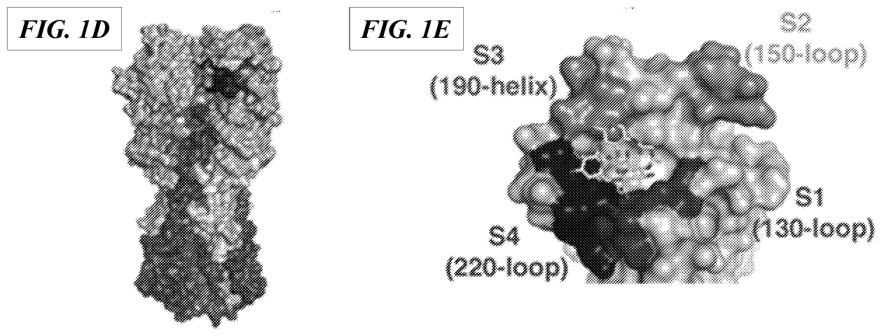

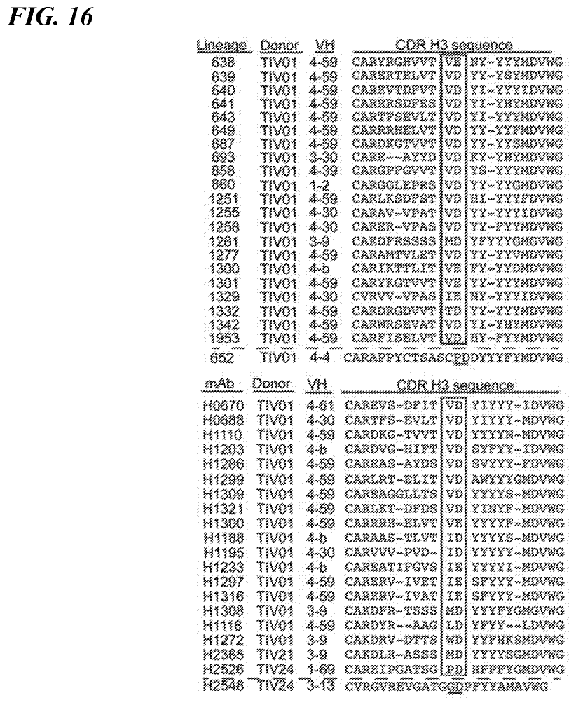

Exemplary sequences for CDR3 of bnAbs that target the RBS are listed in FIG. 1A from Schmidt, et al, Cell 161, 1026-1034, 2015, the contents of which are incorporated herein by reference in its entirety and reproduced herein as FIG. 16.

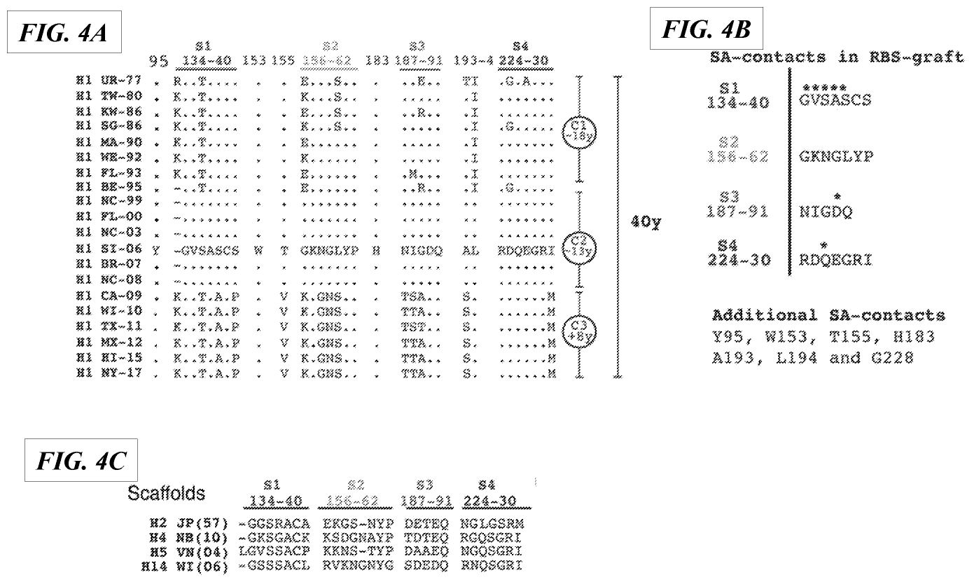

Exemplary conserved regions/epitopes for H1 influenza are provided herein in FIG. 1B, and FIG. 4A.

### Chimeric Epitopes

A chimeric epitope, as that term is used herein, is a fusion protein comprising a hemagglutinin moiety and a neutralizing epitope moiety, each derived from a different viral strain or viral family (i.e., chimeric). In one embodiment, the hemagglutinin is derived from a non-circulating human influenza virus. A non-circulating virus is one that is not currently in circulation in the human population, thus in some embodiments the host will not have a response to the In another embodiment, strain specific epitopes on the wild-type hemagglutinin are not included in the fusion protein.

While the neutralizing epitope moiety can be any desired epitope, it is specifically contemplated herein that the neutralizing epitope is a broadly neutralizing epitope. Thus, in some embodiments, the neutralizing epitope is an epitope found in a conserved region of a virus or among members of a viral family.

A chimeric epitope can be synthesized using well known methods including recombinant methods and chemical synthesis. Recombinant methods of producing a polypeptide through the introduction of a vector including nucleic acid encoding the polypeptide into a suitable host cell are well known in the art, e.g., as described in Sambrook et al., Molecular Cloning: A Laboratory Manual, 2d Ed, Vols 1 to 8, Cold Spring Harbor, N.Y. (1989); M. W. Pennington and B. M. Dunn, Methods in Molecular Biology: Peptide Synthesis Protocols, Vol 35, Humana Press, Totawa, N.J. (1994), contents of both of which are herein incorporated by reference. Peptides can also be chemically synthesized using methods well known in the art. See for example, Merrifield et al., J. Am. Chem. Soc. 85:2149 (1964); Bodanszky, M., Principles of Peptide Synthesis, Springer-Verlag, New York, NY (1984); Kimmerlin, T. and Seebach, D. J. Pept. Res. 65:229-260 (2005); Nilsson et al., Annu. Rev. Biophys. Biomol. Struct. (2005) 34:91-118; W.C. Chan and P. D. White (Eds.) Fmoc Solid Phase Peptide Synthesis: A Practical Approach, Oxford University Press, Cary, N.C. (2000); N. L. Benoiton, Chemistry of Peptide Synthesis, CRC Press, Boca Raton, Fla. (2005); J. Jones, Amino Acid and Peptide Synthesis, 2^{nd} Ed, Oxford University Press, Cary, N.C. (2002); and P. Lloyd-Williams, F. Albericio, and E. Giralt, Chemical Approaches to the synthesis of peptides and proteins, CRC Press, Boca Raton, Fla. (1997), contents of all of which are herein incorporated by reference. Peptide derivatives can also be prepared as described in U.S. Pat. Nos. 4,612,302; 4,853,371; and 4,684,620, and U.S. Pat. App. Pub. No. 2009/0263843, contents of all which are herein incorporated by reference.

### Vaccine Formulation

Chimeric epitopes or compositions comprising chimeric epitopes as described herein, can be resuspended in a solution or buffer (such as, for example, sterile distilled water, saline, phosphate-buffered saline, etc.). In some embodiments, the compositions or vaccines contain no other components. Typically, the composition will be in aqueous form. The composition may include preservatives such as thimerosal or 2-phenoxyethanol. It is preferred, however, that the vaccine should be substantially free from (i.e. less than 5 μg/ml) mercurial material e.g. thimerosal-free. Vaccines containing no mercury are more preferred. Preservative-free vaccines are particularly preferred. α-tocopherol succinate can be included as an alternative to mercurial compounds.

To control tonicity, it is preferred to include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride, calcium chloride, etc.

Compositions can have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. In some embodiments, the composition is allergen free (e.g., gluten free).

Compositions of the invention may include detergent e.g. a polyoxyethylene sorbitan ester surfactant (known as ‘Tweens’), an octoxynol (such as octoxynol-9 (Triton X-100) or -octylphenoxypolyethoxyethanol), a cetyl trimethyl ammonium bromide (‘CTAB’), or sodium deoxycholate. The detergent may be present only at trace amounts. Thus the vaccine may include less than 1 mg/ml of each of octoxynol-10 and polysorbate 80. Other residual components in trace amounts could be antibiotics (e.g. neomycin, kanamycin, polymyxin B).

Influenza vaccines are typically administered in a dosage volume of about 0.5 ml, although a half dose (i.e. about 0.25 ml) may be administered to children.

In some embodiments, the compositions and vaccines described herein can comprise additional components to enhance stability, for example, protease inhibitor(s), osmolarity agents etc. Non-limiting examples of stabilizers include polyethylene glycol, proteins, saccharide, amino acids, inorganic acids, and organic acids which may be used either on their own or as admixtures. Two or more stabilizers can be used in aqueous solutions at the appropriate concentration and/or pH. The specific osmotic pressure in such aqueous solution is generally in the range of 0.1-3.0 osmoses, preferably in the range of 0.80-1.2. The pH of the aqueous solution is adjusted to be within the range of 5.0-9.0, preferably within the range of 6-8.

Alternatively, or additionally, adjuvants can be included in the compositions as described herein. Adjuvants may, in certain embodiments, enhance production of antibodies against one or more influenzaviruses and/or the chimeric epitope. Examples of suitable adjuvants include, but are not limited to, various oil formulations and/or emulsions such as stearyl tyrosine (see, for example, U.S. Pat. No. 4,258,029), muramyl dipeptide (also known as MDP, Ac-Mur-L-Ala-D), saponin, aluminum hydroxide, lymphatic cytokine, QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Alum, and MF59 etc. Adjuvants that are particularly suitable for inducing mucosal immunity include, but are not limited to, cholera toxin B subunit, heat labile enterotoxin (KT) from E. coli, Emulsomes (Pharoms, LTF., Rehovot, Israel), CpG oligodeoxynucleotides (ODNs), Toll-like receptor agonists, polyethyleneimine, chitosan, etc. In some embodiments, multiply mutated form of cholera toxin is used as an adjuvant. In one embodiment, the composition or vaccine further comprises a delivery enhancer, for example, to increase penetration of the mucosal layer (e.g., polyethyleneimine, chitosan etc).

The compositions and vaccines described herein can be formulated for multiple administrations/immunizations, and an effective dose can be achieved by the administration of multiple immunizations whether or not each individual immunization comprises an effective dose.

In some embodiments, the compositions or vaccines are stored in a sealed vial, ampule, or similar container.

In some embodiments, the composition or vaccine is provided in a lyophilized form, which can improve ease in transportation and storage. In some such embodiments, vaccines are dissolved or suspended in a solution or buffer before administration.

In some embodiments, the composition or vaccine further comprises additional therapeutic agents (such as other vaccines or antigens associated with other diseases). In some embodiments, the other therapeutic agents do not diminish effectiveness of the vaccine composition for inducing immunity against influenza. In some embodiments, the vaccine or composition is administered in combination with other therapeutic ingredients including, e.g., interferons, cytokines, or chemotherapeutic agents. In some embodiments, the vaccine composition as disclosed herein can be administered with one or more co-stimulatory molecules and/or adjuvants as disclosed herein.

In one embodiment, the composition or vaccine as described herein comprises pharmaceutically acceptable carriers that are inherently nontoxic and nontherapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. For all administrations, conventional depot forms are suitably used. Such forms include, for example, microcapsules, nano-capsules, liposomes, plasters, inhalation forms, nose sprays, sublingual tablets, and sustained release preparations. For examples of sustained release compositions, see U.S. Pat. Nos. 3,773,919, 3,887,699, EP 58,481A, EP 158,277A, Canadian Patent No. 1176565; U. Sidman et al., Biopolymers 22:547 (1983) and R. Langer et al., Chem. Tech. 12:98 (1982).

In one embodiment, other ingredients can be added to vaccine formulations, including antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

The compositions and vaccines described herein can be formulated for any of a variety of routes of administration as discussed further below. For example, the compositions or vaccines can be formulated as a spray for intranasal inhalation, nose drops, swabs for tonsils, etc. The compositions or vaccines can be formulated for oral delivery in the form of capsules, tablets, gels, thin films, liquid suspensions and/or elixirs, etc. In one embodiment, the composition or vaccine is formulated for sublingual administration.

In some embodiments, the immunogenic compositions as described herein can be administered intravenously, intranasally, intramuscularly, subcutaneously, intraperitoneally, sublingually, vaginal, rectal or orally. In some embodiments, the route of administration is oral, intranasal, subcutaneous, or intramuscular. In some embodiments, the route of administration is sublingual, nasal, or oral administration.

When oral preparations are desired, the vaccine compositions can be combined with typical carriers, such as lactose, sucrose, starch, talc magnesium stearate, crystalline cellulose, methyl cellulose, carboxymethyl cellulose, glycerin, sodium alginate or gum arabic among others.

For some formulations (i.e., i.v. injection), the immunogenic compositions as described herein for administration must be sterile. Sterility is readily accomplished by filtration through sterile filtration membranes, or by gamma radiation.

In some embodiments, the vaccine composition is administered in a pure or substantially pure form, but it is preferable to present it as a pharmaceutical composition, formulation or preparation. Such formulation comprises decorated bacteria as described herein together with one or more pharmaceutically acceptable carriers and optionally other therapeutic ingredients.

### Target Populations for Vaccination

Ideally, the target population for vaccination with a flu vaccine as described herein includes the entire population, e.g., healthy young adults (e.g. aged 18-60), elderly (typically aged above 60) or infants/children. A target sub-population includes those individuals at the highest risk of mortality or complications arising from the flu, for example, immuno-compromised individuals, children and the elderly. Immuno-compromised humans generally are less well able to respond to an antigen, in particular to an influenza antigen, in comparison to healthy adults.

In one aspect, the target population is a population which is unprimed against influenza, either being naive (such as vis à vis a pandemic strain), or having failed to respond previously to influenza infection or vaccination. In other embodiments, the target population comprises elderly persons, for example, those over at least 60, or 65 years and over, younger high-risk adults (i.e. between 18 and 60 years of age) such as people working in health institutions, or those young adults with a risk factor such as cardiovascular and pulmonary disease, or diabetes. Another target population is all children 6 months of age and over, who experience a relatively high influenza-related hospitalization rate. In particular, the flu vaccines described herein are suitable for pediatric use in children between 6 months and 3 years of age, or between 3 years and 8 years of age, such as between 4 years and 8 years of age, or between 9 years and 17 years of age.

### Dosage, Administration and Efficacy

The compositions and methods described herein can be administered to a subject in need of vaccination, immunization, and/or stimulation of an immune response. In some embodiments, the methods described herein comprise administering an effective amount of compositions described herein, e.g., to a subject in order to stimulate an immune response or provide protection against one or more influenza viruses from which it was derived. Providing protection against the influenza virus(es) is stimulating the immune system such that later exposure to the antigen (e.g., on or in a live pathogen) triggers a more effective immune response than if the subject was naïve to the antigen. Protection can include faster clearance of the pathogen, reduced severity and/or time of symptoms, and/or lack of development of disease or symptoms. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, injection, or topical, administration. Administration can be local or systemic. In some embodiments of any of the aspects, the administration can be intramuscular or subcutaneous.

The term “effective amount” as used herein refers to the amount of adjuvant needed to stimulate the immune system, or in combination with an antigen, to provide a protective effect against subsequent infections, and relates to a sufficient amount of pharmacological composition to provide the desired effect. The term “therapeutically effective amount” therefore refers to an amount of the adjuvant (and optionally, the antigen) that is sufficient to provide a particular immune stimulatory effect when administered to a typical subject. An effective amount as used herein, in various contexts, would also include an amount sufficient to delay the development of a symptom of the disease, alter the course of a symptom of the disease (for example but not limited to, slowing the progression of a symptom of the disease), or prevent a symptom of the disease. Thus, it is not generally practicable to specify an exact “effective amount”. However, for any given case, an appropriate “effective amount” can be determined by one of ordinary skill in the art using only routine experimentation.

As will be appreciated by one of skill in the art, appropriate dosing regimens for a given composition or vaccine can comprise a single administration/immunization or multiple ones. For example, vaccines can be given as a primary immunization followed by one or more boosters. Boosters may be delivered via the same and/or different route as the primary immunization. Boosters are generally administered after a time period following the primary immunization or the previously administered booster. For example, a booster can be given about two weeks or more after a primary immunization, and/or a second booster can be given about two weeks or more after the first boosters. Boosters may be given repeatedly at time periods, for example, about two weeks or greater throughout up through the entirety of a subject's life. Boosters can be spaced, for example, about two weeks, about three weeks, about four weeks, about one month, about two months, about three months, about four months, about five months, about six months, about seven months, about eight months, about nine months, about ten months, about eleven months, about one year, about one and a half years, about two years, about two and a half years, about three years, about three and a half years, about four years, about four and a half years, about five years, about ten years, about 20 years, about 30 years or more after a primary immunization or after a previous booster.

The precise dose to be employed in the formulation will also depend on the route of administration and should be decided according to the judgment of the practitioner and each patient's circumstances. Ultimately, the attending physician will decide the amount of protein or vaccine composition to administer to particular individuals.

Vaccination can be conducted by conventional methods. For example, a polypeptide can be used in a suitable diluent such as saline or water, or complete or incomplete adjuvants. The vaccine can be administered by any route appropriate for eliciting an immune response (e.g., sublingual, nasal, oral or intramuscular injection). The vaccine can be administered once or at periodic intervals until an immune response is elicited. Immune responses can be detected by a variety of methods known to those skilled in the art, including but not limited to, antibody production, cytotoxicity assay, proliferation assay and cytokine release assays. For example, samples of blood can be drawn from the immunized mammal, and analyzed for the presence of antibodies against the antigens of the immunogenic composition by ELISA (see de Boer G F, et. al., 1990, Arch Virol. 115:47-61) and the titer of these antibodies can be determined by methods known in the art.

In one embodiment, efficacy is determined by measuring the immunogenicity of the administered composition or vaccine, for example, by assessing immunity to the individual to which the composition is administered or immunity conferred to one or more offspring of the individual to which the composition is administered. For example, the individual being administered the composition can be a pregnant female, whose future or current offspring benefit from immune protection. Such immunity can be passed from mother to child, for example, through breastmilk and/or through blood exchanged between from mother and fetus via the placenta.

In some embodiments, antibody titer can be used as a measure of the humoral immunogenicity of a given composition or vaccine. As used herein, antibody titer is a measurement of how much antibody an organism, such as, for example, a human, a mouse or a rabbit, has produced that recognizes a particular epitope, expressed as the greatest dilution that still gives a positive result. ELISA is a common means of determining antibody titers, but other assays known to one of skill in the art can be used as well.

In other embodiments, efficacy can be determined by assessing a variety of clinical measures including, but not limited to, fewer cases of influenza than expected in a given population, a reduction in the severity of influenza, reduced number of hospitalizations, multi-year immunity to influenza viruses, multi-strain immunity etc.

The efficacy of a given composition for inducing immunity to one, two or more influenza viruses can be determined by the skilled clinician. However, a composition is considered “effective,” as the term is used herein, if any one or all of the signs or symptoms of the disease (e.g., flu) is/are altered in a beneficial manner, or other clinically accepted symptoms or markers of disease are improved, or ameliorated, e.g., by at least 10% in individuals administered the composition compared to a substantially similar individual that has not been administered or immunized as described herein. Efficacy can also be measured by failure of an individual to worsen as assessed by stabilization of the disease (e.g., shorter duration, less intense symptoms), or the need for medical interventions (i.e., progression of the disease is halted or at least slowed). Methods of measuring these indicators are known to those of skill in the art and/or described herein.

In some embodiments, the subject is further evaluated using one or more additional diagnostic procedures, for example, by medical imaging, physical exam, laboratory test(s), clinical history, family history, gene tests, and the like. Medical imaging is well known in the art. As such, the medical imaging can be selected from any known method of imaging, including, but not limited to, ultrasound, computed tomography scan, positron emission tomography, photon emission computerized tomography, and magnetic resonance imaging.

### Kits

Kits for the preparation of a composition comprising a chimeric epitope as described herein or for vaccine administration are provided herein. At a minimum, the kit will comprise one or more chimeric epitopes and instructions for use therefor.

Compositions and vaccines as described herein can be prepared extemporaneously (e.g., at the time of delivery) particularly when an adjuvant is being used. Thus, in some embodiments, a kit provided herein comprises various components ready for mixing of a composition. For example, the kit can allow the adjuvant and the antigen to be kept separately until the time of use.

The components are physically separate from each other within the kit, and this separation can be achieved in various ways. For instance, the two components may be in two separate containers, such as vials. The contents of the two vials can then be mixed e.g. by removing the contents of one vial and adding them to the other vial, or by separately removing the contents of both vials and mixing them in a third container.

In a preferred arrangement, one of the kit components is in a syringe and the other is in a container such as a vial. The syringe can be used (e.g., with a needle) to insert its contents into the second container for mixing, and the mixture can then be withdrawn into the syringe. The mixed contents of the syringe can then be administered to a patient, typically through a new sterile needle. Packing one component in a syringe eliminates the need for using a separate syringe for patient administration.

The kit components will generally be in aqueous form. In some arrangements, a component (typically a chimeric epitope component rather than an adjuvant component) is in dry form (e.g. in a lyophilized form), with the other component being in aqueous form. The two components can be mixed in order to reactivate the dry component and give an aqueous composition for administration to a patient. A lyophilised component will typically be located within a vial rather than a syringe. Dried components can include stabilizers such as lactose, sucrose or mannitol, as well as mixtures thereof e.g. lactose/sucrose mixtures, sucrose/mannitol mixtures, etc. One possible arrangement uses an aqueous adjuvant component in a pre-filled syringe and a lyophilised antigen component in a vial.

Suitable containers for compositions of the invention (or kit components) include vials, syringes (e.g. disposable syringes), nasal sprays, etc. These containers are preferably sterile.

Where a composition/component is located in a vial, the vial is preferably made of a glass or plastic material. The vial is preferably sterilized before the composition is added to it. To avoid problems with latex-sensitive patients, vials are preferably sealed with a latex-free stopper, and the absence of latex in all packaging material is preferred. The vial may include a single dose of vaccine, or it may include more than one dose (a ‘multidose’ vial) e.g. 10 doses. Preferred vials are made of colorless glass.

A vial can have a cap (e.g. a Luer lock) adapted such that a pre-filled syringe can be inserted into the cap, the contents of the syringe can be expelled into the vial (e.g. to reconstitute lyophilised material therein), and the contents of the vial can be removed back into the syringe. After removal of the syringe from the vial, a needle can then be attached and the composition can be administered to a patient. The cap is preferably located inside a seal or cover, such that the seal or cover has to be removed before the cap can be accessed. A vial may have a cap that permits aseptic removal of its contents, particularly for multidose vials.

Where a component is packaged into a syringe, the syringe may have a needle attached to it. If a needle is not attached; a separate needle may be supplied with the syringe for assembly and use. Such a needle may be sheathed. Safety needles are preferred. 1-inch 23-gauge, 1-inch 25-gauge and ⅝-inch 25-gauge needles are typical. Syringes may be provided with peel-off labels on which the lot number, influenza season and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of vaccine. The syringe will generally have a tip cap to seal the tip prior to attachment of a needle, and the tip cap is preferably made of a butyl rubber. If the syringe and needle are packaged separately then the needle is preferably fitted with a butyl rubber shield. Useful syringes are those marketed under the trade name “Tip-Lok”™.

Containers may be marked to show a half-dose volume e.g. to facilitate delivery to children. For instance, a syringe containing a 0.5 ml dose may have a mark showing a 0.25 ml volume.

Where a glass container (e.g. a syringe or a vial) is used, then it is preferred to use a container made from a borosilicate glass rather than from a soda lime glass.

A kit or composition may be packaged (e.g. in the same box) with a leaflet including details of the vaccine e.g. instructions for administration, details of the antigens within the vaccine, etc. The instructions may also contain warnings e.g. to keep a solution of adrenaline readily available in case of anaphylactic reaction following vaccination, etc.

The present invention can be defined in any of the following number paragraphs:

1. A chimeric epitope comprising:
i. a conserved donor receptor binding site (RBS) or a neutralizing epitope, or a functional fraction thereof; and
ii. an acceptor molecular scaffold or fragment thereof

2. The chimeric epitope of paragraph 1, wherein the RBS or the neutralizing epitopes are fused to the molecular scaffold.

3. The chimeric epitope of any of the preceding paragraphs, wherein the RBS is a conserved epitope on the influenza virus hemagglutinin (HA).

4. The chimeric epitope of any of the preceding paragraphs, wherein the acceptor molecular scaffold is an antigenically distinct HA.

5. The chimeric epitope of any of the preceding paragraphs, wherein the HA comprises an HA without the RBS or the neutralizing epitopes.

6. The chimeric epitope of any of the preceding paragraphs, wherein the donor RBS or the neutralizing epitope and the acceptor molecular scaffold are derived from a family of viruses selected from the group consisting of: Arenaviridae, Bunyaviridae, Coronaviridae, Filoviridae, Flaviviridae, Orthomyxoviridae, Paramyxoviridae, Pneumoviridae, and Retroviridae.

7. The chimeric epitope of any of the preceding paragraphs, wherein the donor RBS and the acceptor molecular scaffold are derived from the family Orthomyxoviridae.

8. The chimeric epitope of any of the preceding paragraphs, wherein the donor RBS and the acceptor molecular scaffold are derived from the same viral family.

9. The chimeric epitope of any of the preceding paragraphs, wherein the donor RBS and the acceptor molecular scaffold are derived from a different viral family.

10. The chimeric epitope of any of the preceding paragraphs, wherein the donor RBS and the acceptor molecular scaffold are derived from the same viral family but different antigenic viral types.

11. The chimeric epitope of any of the preceding paragraphs, wherein the donor RBS and the acceptor molecular scaffold are derived from the same viral family but different host of origin.

12. The complex epitope of any of the preceding paragraphs, wherein the donor RBS and the acceptor molecular scaffold are derived from the same viral family but different geographical origin.

13. The chimeric epitope of any of the preceding paragraphs, wherein the donor RBS and the acceptor molecular scaffold are derived from the same viral family but different viral strains or subtypes.

14. The chimeric epitope of any of the preceding paragraphs, wherein the donor RBS and the acceptor molecular scaffold are derived from the same viral family but different year of isolation.

15. The chimeric epitope of any of the preceding paragraphs, wherein the donor RBS is the RBS of circulating, previously circulating, or pre-pandemic influenzas viruses

16. The chimeric epitope of any of the preceding paragraphs, circulating or previously circulating influenzas are H1, H2, H3 or B.

17. The chimeric epitope of any of the preceding paragraphs, pre-pandemic influenza viruses are H5, H7 and H9 influenzas.

18. The chimeric epitope of any of the preceding paragraphs, wherein the RBS is an RBS of H1 influenza was isolated in 1918-present day.

19. The chimeric epitope of any of the preceding paragraphs, wherein the RBS of H1 influenza is H1/Massachusetts/1/1990; H1/Solomon Islands/3/2006; or H1/California/04/2009 or a variant thereof.

20. The chimeric epitope of any of the preceding paragraphs, wherein the molecular scaffold has substantially no preexisting immunity in the population of a subject.

21. The chimeric epitope of any of the preceding paragraphs, wherein the molecular scaffold does not boost a strain-specific response.

22. The chimeric epitope of any of the preceding paragraphs, wherein the molecular scaffold is derived from H2, H4, H5, H6, H7, H8, H9, H10, H11, H12, H13, H14, H15, H16, H17 or H18 influenzas.

23. The chimeric epitope of any of the preceding paragraphs, wherein the molecular scaffold is derived from group 1 influenzas or group 2 influenzas.

24. The chimeric epitope of any of the preceding paragraphs, wherein group 1 influenza is selected from the group consisting of: H2N2 A/Japan/305/1957; H5N8 A/gyrfalcon/Washington/41088-6/2014; H6N8 A/widgeon/Wisconsin/617/1983; H9N2 A/swine/Hong Kong/9/1998; and H16N3 A/laughing-gull/Delaware Bay/296/1998.

25. The chimeric epitope of any of the preceding paragraphs, wherein group 2 influenza is selected from the group consisting of: H3N2 A/Aichi/2/1968; H4N6 A/America black duck/New Brunswick/00464/2010; H7N9 A/Shanghai/1/2013;, H10N7 A/mallard/Wisconsin/1350/1983; and H14N6 A/mallard/Wisconsin/10OS3941/2010.

26. A chimeric epitope comprising;
i. a conserved donor receptor binding site (RBS) derived from circulating H1 influenza; and
ii. an acceptor molecular scaffold derived from non-circulating influenza.

27. The chimeric epitope of any of the preceding paragraphs, wherein the circulating H1 influenza is H1/Massachusetts/1/1990; H1/Solomon Islands/3/2006; or H1/California/04/2009 or variant thereof.

28. The chimeric epitope of any of the preceding paragraphs, wherein the non-circulating influenza is H6N8 A/widgeon/Wisconsin/617/1983; H16N3 A/laughing-gull/Delaware Bay/296/1998; H4N6 A/America black duck/New Brunswick/00464/2010; or H14N6 A/mallard/Wisconsin/10OS3941/2010 or any influenza subtype.

29. The chimeric epitope of any of the preceding paragraphs, wherein the molecular scaffold exhibits substantially no preexisting immunity to circulating influenzas.

30. The chimeric epitope of any of the preceding paragraphs, wherein the molecular scaffold is engineered to comprise at least one amino acid mutation.

31. The chimeric epitope of any of the preceding paragraphs, wherein the at least one amino acid mutation is selected from the group consisting of: N145S, T192R, S193A, K196H, A198E and S219K.

32. An immunogenic composition comprising; the chimeric epitope of paragraphs 1-31 and a pharmaceutically acceptable carrier.

33. The immunogenic composition of any of the preceding paragraphs, wherein the composition is used to elicit an immune response in a subject.

34. The immunogenic composition of any of the preceding paragraphs, for the use of a diagnostic for exposure to a pathogen or immune threat.

35. The immunogenic composition of any of the preceding paragraphs, wherein the composition is used to prevent an infection caused by a pathogen in a subject.

36. The immunogenic composition of any of the preceding paragraphs, wherein the infection is an influenza infection.

37. The immunogenic composition of any of the preceding paragraphs, for the use of vaccinating a subject.

38. A method for inducing an immune response in a subject, the method comprising;
administering to a subject the chimeric epitope of any of the preceding paragraphs.

39. A method for vaccinating a subject, the method comprising; administering to a subject the chimeric epitope of any of the preceding paragraphs.

40. A method for inducing an immune response in a subject, the method comprising;
administering to a subject the immunogenic composition of any of the preceding paragraphs.

41. A method for vaccinating a subject, the method comprising; administering to a subject the immunogenic composition of any of the preceding paragraphs.

42. The method of any of the preceding paragraphs, wherein the subject is human.

43. The method of any of the preceding paragraphs, wherein the subject is an agricultural or non-domestic animal.

44. The method of any of the preceding paragraphs, wherein the subject is a domestic animal.

45. The method of any of the preceding paragraphs, wherein the subject is a bird.

46. The chimeric epitope of any of the preceding paragraphs, wherein the RBS and the molecular scaffold are a mammalian RBS and the molecular scaffold.

47. The chimeric epitope of any of the preceding paragraphs, the RBS and the molecular scaffold are a human RBS and the molecular scaffold.

48. The chimeric epitope any of the preceding paragraphs, the RBS and the molecular scaffold are a bird RBS and the molecular scaffold.

### EXAMPLES

Provided herein are compositions using a structural understanding of hemagglutinins (HAs) to pursue a structure-based immunogen design strategy and a stepwise approach to focusing the humoral response to the receptor binding site (RBS). These design efforts specifically ask whether the immune system can be manipulated in a deterministic way to increase the frequency of RBS-directed antibodies in both naïve and immune-experienced conditions. These studies use structure-guided “resurfacing” of non-circulating HAs to serve as molecular scaffolds to present the conserved RBS “core” from circulating and potential pandemic influenzas. These “acceptor” HA scaffolds have limited preexisting immunity within the population and therefore significantly reduce memory recall of strain-specific responses targeting the immunodominant head. Furthermore, the heterologous RBS periphery of the scaffolds reinforce humoral responses to conserved RBS core. Multimerization through engineered disulfides in head-only constructs conceal neo-epitopes present on monomers and increased valency potently stimulate BCRs. In some embodiments, the designed immunogens can be tested for in vivo efficacy using the inventors' novel murine “Nojima cultures” method to rapidly screen and characterize immune responses. Such strategies provide candidate immunogens that focus responses to the conserved RBS. This epitope scaffolding approach can serve as a vaccine platform for other rapidly evolving pathogens where antigenic diversity can be exploited for immunogen design strategies.

### Example 1

As described herein, the complex, broadly neutralizing epitope of the RBS from circulating H1 influenza has been grafted onto non-circulating influenzas in order to focus the immune response to conserved epitopes, and to overcome preexisting immunity present in the population. These “acceptor”, resurfaced HAs are molecular scaffolds that present the “donor” conserved H1 RBS core and remove epitopes targeted by strain-specific responses in immune-experienced individuals. The crystal structure of one resurfaced HA (rsHA) in complex with a broadly neutralizing antibody (bnAb) has been determined. Through structure-guided optimization, two independent molecular scaffolds were improved to bind a diverse panel of bnAbs targeting the RBS. Strategies described herein provide candidate immunogens for a universal flu vaccine, by exploiting the immunogenicity of the conserved RBS.

### Example 2A

### Grafting a Complex, Broadly Neutralizing Epitope onto a Protein Scaffold

Non-circulating, hemagglutinins (HAs) were used as Nature's own “molecular scaffolds” to present the conserved receptor binding site (RBS) of circulating H1 influenzas. This approach circumvents the considerable hurdle of de novo design of a molecular scaffold that displays the complex, conformationally-specific RBS epitope. The H1 RBS graft supply the core residues necessary for binding a diverse panel of broadly neutralizing, RBS-directed antibodies. The HA scaffolds have no preexisting immunity in the human population and thus prevents boosting of strain-specific, non-protective responses in influenza-experienced individuals. These scaffolds may serve as candidate immunogens for a broadly protective (“universal”) influenza vaccine.

Additionally, the presentation of the RBS in immunogens will both redirect and direct immune responses in immune-experienced individuals to this site; any preexisting anti-influenza response is refined to a conserved, universal epitope while adapting to the heterologous periphery of the HA scaffold. Thus, a once strain-specific response is converted to a broadly neutralizing, protective response. These immunogens will suppress boosting of preexisting, strain-specific humoral responses by using non-circulating HAs as scaffolds and boost RBS-directed, broadly protective responses. Additionally, the inventors have developed and implemented a novel single-B cell method, “Nojima cultures”, allowing for an unprecedented characterization of BCR phenotypes and genotypes for large numbers (>10⁴) of individual B-cells. This method permits rapid screening, characterization and subsequently refines the designed immunogens using iterative structure-based, directed evolution and biochemical approaches. Furthermore, Nojima analysis permits precise quantification of Abs directed to the RBS by these immunogens and permits direct comparison to their improvement over the standard seasonal vaccine. This will provide invaluable insight into showing how the immune system can be directed to a subdominant epitope.

This type of analysis has not been previously done. Current research for a broadly protective influenza vaccine has focused almost exclusively on stem-directed bnAbs while ignoring the necessary and critical contribution of bnAbs to the RBS. Alternatively, this approach implements protein engineering strategies to design immunogens to focus to this RBS epitope. Importantly, this grafting approach can synergize with other current influenza-vaccine design approaches such as COBRA and chimeric HAs by grafting the conserved RBS onto these immunogens resulting in optimized immunogens that target multiple conserved epitopes.

The complex epitope of the receptor binding site (RBS) from one influenza hemagglutinin (HA) can be grafted to another, antigenically distinct HA. The acceptor HA acts as a molecular scaffold presenting the conserved RBS from circulating (e.g. H1, H3) and potential pandemic subtypes (e.g. H5, H7). Use of acceptor, scaffold HAs that have not previously circulated in the human population reduce boosting of strain-specific responses in influenza-experienced conditions.

The RBS of influenza HA coordinates sialic acid (SA) which is required for cell entry and necessary to establish infection. Despite overall sequence diversity within the HA subtypes (18 total) all are optimized within the RBS “core” to bind sialic acid. This RBS core includes residues that are strictly conserved across HA subtypes. The RBS core is a complex, conformationally-specific epitope involving multiple segments, separated in linear sequence, but adjacent in conformational space. Antibodies (Abs) targeting the RBS are often broadly neutralizing because they contact invariant core residues. Eliciting this class of bnAbs is a goal of a universal influenza vaccine. Immunogens designed to elicit Abs targeting this complex epitope should present it in the correct conformation; immunization with linear peptide(s) comprising the epitope may not suffice. Because the overall RBS architecture of each HA subtype is optimized to engage sialic acid, it was hypothesized that the RBS segments from one subtype are transplantable onto a different subtype circumventing the necessity of de novo scaffold design.

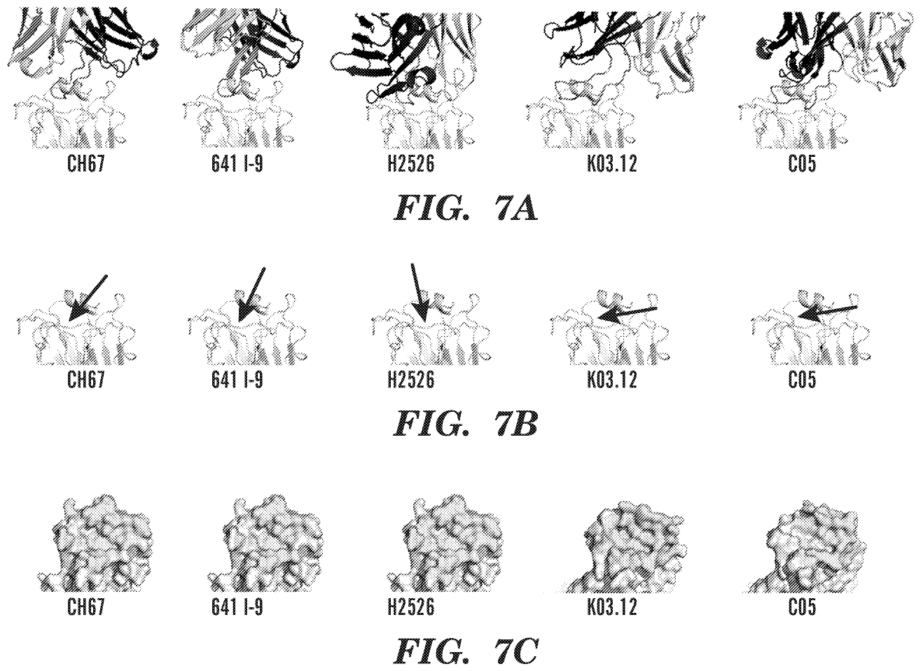

Grafting a representative HI RBS onto different HA subtypes. As a proof-of-principle, previously circulating H1 A/Solomon Islands/3/2006 (H1 SI-06) was selected as the initial donor RBS graft. Four “segments”, S1-S4 were defined that include 7 of the 13 critical SA-contacting residues. Many of the remaining residues not included in the graft, are in the base of the RBS and are conserved/nearly invariant across subtypes. For the initial acceptor scaffolds the H4 and H14 subtypes were selected because they are antigenically distinct having little conservation to the donor HA and there are crystal structures of the wildtype HAs (PDBs 5XL3 and 3EYJ, respectively) that could serve as a structure-based guide. The strains H4N6 A/America black duck/New Brunswick/00464/2010 and H 14N6 A/mallard/Wisconsin/10OS3941/2010 were selected. The acceptor scaffold boundaries, S1-S4, were defined by aligning the H1 SI-06 sequence and through structural analysis. Grafting was deemed successful if 1) the rsHA protein could be over-expressed in mammalian and/or insect cells, and 2) the rsHA binds to the panel of RBS-directed bnAbs (FIGS. 7A, 7C). This bnAb panel represents the polyclonal, RBS-directed response desired by a universal influenza vaccine. This panel was used to characterize and validate the immunogens described herein. The inventors successfully over expressed monomeric rsHA “heads” (residues 37-319) and trimeric full length soluble ectodomains (FLsEs) of rsH4 and rsH14 HAs with the H1 SI-06 RBS.

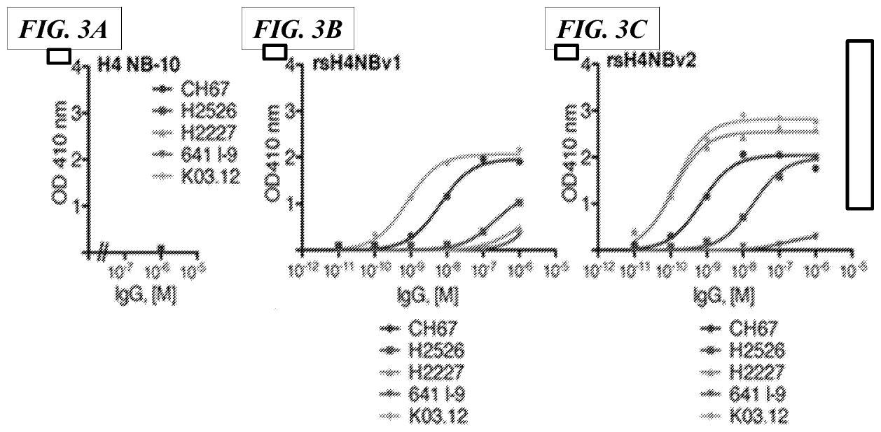

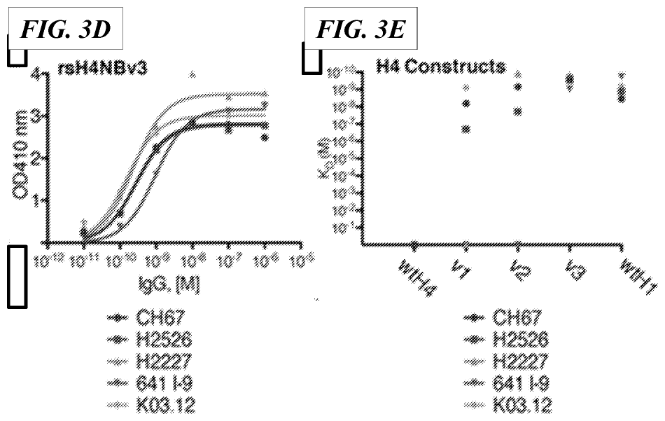

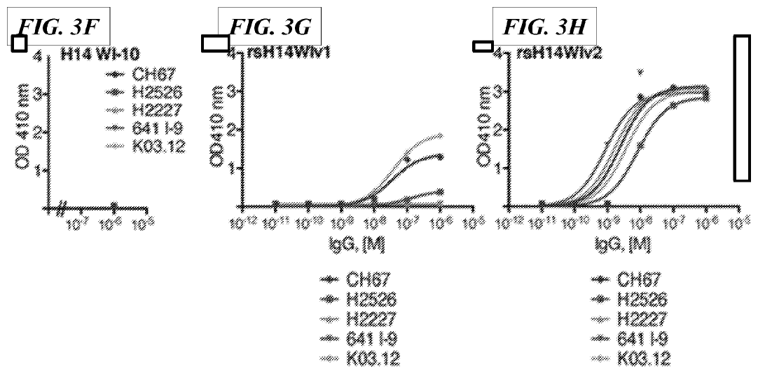

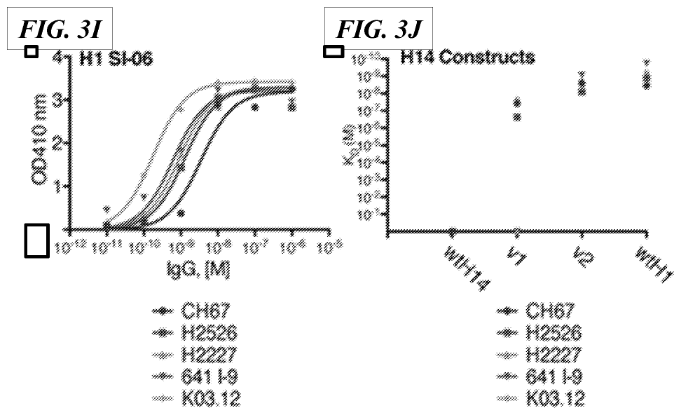

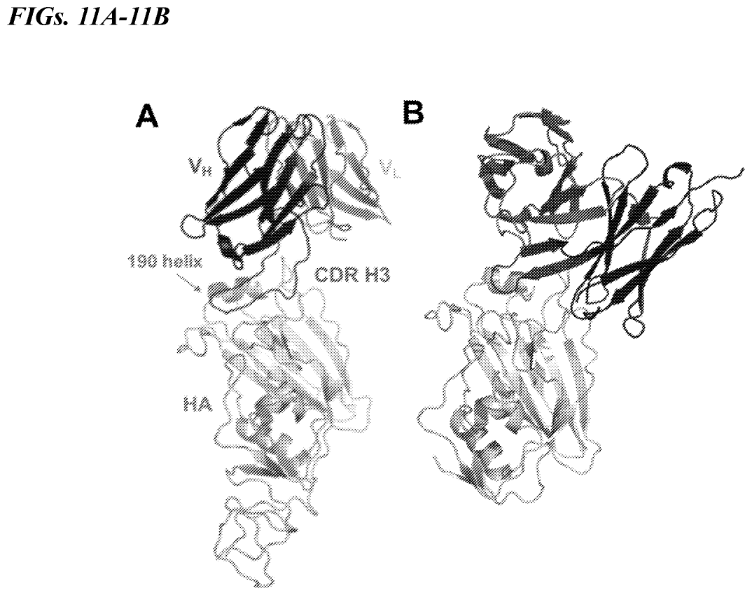

These first generation rsHAs were validated by assaying for binding to the panel of H1 and H1, H3 cross-reactive RBS-directed antibodies using biolayer interferometry (BLI). As seen in Table 1, the first generation rsH4NBv1 and rsH14WIv1 bound only 1 of 6 and 3 of 6 bnAbs, respectively; none bound to the same affinity as the wildtype H1 SI-06 which the graft is based upon. (Note: Affinities in Table 1 are binding of a monomer head and an Fab. These measurements avoid inconsistencies due to “avidity” effects of a dimeric IgG and/or a trimeric HA. In general, the avidity (effective KD) of an IgG for trimeric HA will be at least 10² to 10³ tighter (i.e., smaller KD) than the monomer-monomer value.) These preliminary data indicate the feasibility of this grafting approach by identifying acceptor scaffolds and showing conferred binding to RBS-directed bnAbs. However, the lack of binding to the entire panel of RBS-directed bnAbs highlights the nuances associated with our approach and the subsequent “finessing” of the grafts necessary for correct epitope presentation. To that end, a crystal structure of the first generation scaffold rsH4NBv1 in complex with the bnAb K03.12 (FIG. 11). Structure-based modifications were then made to the scaffold based on docking bnAbs from FIGS. 7A-7C in order to alleviate steric clashes and/or reinforce interactions. Two subsequent iterations, v2 and v3, were then assayed for binding in BLI (Table 1) or ELISA (FIGS. 3A-3E). The scaffold was improved in an iterative fashion resulting in an optimized rsH4NBv3 that interacts with all bnAbs in this panel as well as, or even better than, the wildtype H1 SI-06.

Grafting currently circulating H1, H3 and pre-pandemic RBS onto acceptor scaffolds. Based on the initial success with H1 SI-06 grafting, the same strategy can be pursued to graft the RBS from currently circulating H1 and H3 viruses and extend to potential pandemic H5 and H7 HAs. H1 influenzas evolved in the human population between 1977-2009, little variation occurred in the RBS donor segments until the introduction of the new pandemic H1 California/04/2009. Since 1968 circulating H3s have varied more significantly during their punctuated evolution (FIG. 4A). Despite this variation, bnAbs have been identified for both Hls (e.g. CH67) and H3s (e.g. K03.12, C05) that can accommodate these variations (FIGS. 7A-7C). It is specifically contemplated herein that this approach can be used with H3N2 A/Singapore/INFIMH-16-0019/2016 and H1N1 A/Michigan/45/2015 as donors.

Iterative improvement and optimization of rsHAs scaffolds for presentation of donor grafts. As noted, first generation scaffolds did not initially confer high affinity binding to the entire panel of RBS-directed bnAbs. Additional scaffold modifications in the surrounding RBS periphery were necessary to 1) present the “correct” RBS architecture and/or 2) alleviate potential steric clashes with RBS-directed antibodies. The three strategies outlined below can iteratively improve the rsHA scaffolds through structural, directed evolution and biochemical approaches. The desired affinity (K_{D}) threshold of the rsHAs for the bnAb panel is set to ˜1 μM, with an optimal dissociation half-life of >1s. This threshold was set for monomer:monomer interactions (rsHA head:Fab) knowing that a divalent IgG will have an affinity ˜10 nM. This ensures that the immunogens are well-below the activating threshold when tested in vivo. All kinetic parameters (KD, ka and koff) are obtained using BLI. The immunogens are “affinity matured” to bind each of the RBS-directed Abs to this affinity threshold.

Structural characterization of rsHA and bNAbs immunogens for construct improvement. X-ray structural analyses of the rsHAs can be performed, both independently and in complex with bnAbs. In both cases the rsHA:Ab complex is compared with previously characterized wildtype HA:Ab complexes. The benefits of these structural analyses are two-fold: i) it ensures the grafted RBS is structurally similar to the wildtype RBS and ii) they may suggest mutations to optimize interactions with the panel of RBS-directed Abs.

Iterative improvement of rsHA scaffolds through directed evolution. Iterative rounds of protein-directed evolution can be used to increase the affinity of the rsHAs to the panel of RBS-directed bnAbs. It is specifically contemplated that yeast display can be used as a directed evolution approach. This approach was used to display the HA1 protein from wildtype H1 SI-06 and H3 AI-68 on the yeast cell-surface (data not shown). Mutagenized libraries of the rsHA constructs can be produced in both an unbiased and a targeted approach to maximize diversity and increase the possibility of selecting high-affinity antigens. Initially, a randomly mutagenized library over the entire rsHA construct can be produced. This will select for residues both in the antigen-combining site and periphery that may contribute allosterically to increasing the overall affinity. Second, saturated mutagenesis adjacent to the donor grafts is used. Briefly, these libraries can be made using mutagenic PCR in the presence of 8-oxo-dGTP and dPTP. Subsequent libraries are passaged so that only one mutagenized plasmid per yeast cell is maintained. Random clones can be sequenced to confirm degree of mutagenesis. Selection begins with two rounds of MACS followed by additional rounds of FACS (as needed) using the RBS-directed panel of Fabs conjugated to magnetic beads (for MACS) or fluorescently labeled IgGs (for FACS). The enriched clones are sequenced to identify the mutation(s) imparting the increased affinity. The mutagenized rsHAs are shuttled to mammalian or insect expression vectors and expressed as described above. Their increased affinities for the panel of RBS-directed Abs confirmed using BLI.

Scaffold optimization using sequence conservation and segmental analysis. It is contemplated herein that successful acceptor scaffolds can be used as templates to identify a common consensus scaffold that is optimal for presenting the H1 or H3 grafts. For example, if it is determined that an H10 cannot accept the H1 RBS graft, one would then 1) identify the conserved residues common in the successful H4 and H14 scaffolds and 2) through alignment to wildtype H10, engineer these consensus residues into the H10 scaffold. Through inspection of high-resolution wildtype HA structures, the residues that are likely to have an effect on the conformation of the RBS can be identified and prioritized for construct synthesis and expression.

In a second approach, a pairwise-segmental analysis can be performed. Initial grafts, as described herein, involved complete transfer of segments S1-S4. A step-wise pairing (e.g. S1 with S2, S1 with S3, etc.) can be used to determine if successful segmental pairing(s) on an acceptor HA can be found. In the remaining, non-transplantable segment(s) single amino-acid substitution can be performed sequentially to determine tolerant positions. These efforts are to specifically modify the rsHA for optimized RBS-directed binding. Understanding which segments can be independently tolerated through grafting or which additional mutations are necessary to augment proper RBS display will likely improve the affinity of rsHAs to the panel of RBS-directed Abs.

Functional and biochemical characterization of rsHAs. A series of functional assays can be used to characterize the rsHAs. 1) Antigenicity: using both RBS-directed bnAbs and Abs whose footprints are outside the RBS. The latter will depend on the acceptor HA for Ab availability. BLI can be used to obtain K_{D}s. Reactivity of the rsHAs to these Abs will also be a read out of proper protein folding. 2) Stability: differential scanning calorimetry (DSC) can be performed to assess the stability of the rsHAs in reference to their wildtype counterparts. For each rsHA (e.g. H6N8 A/widgeon/Wisconsin/617/1983) wildtype version has been synthesized for direct comparison. 3) Receptor binding: commercially available glycan microarrays can be used to characterize the affinities and specificities of the resurfaced proteins for sialic acids. These functional and biochemical tests will determine if one has retained, changed or broadened receptor specificities of rsHAs.

### Example 2B

### Oligomerization of Head-Only rsHA Immunogens to Elicit Broad, Protective Responses

It was hypothesized that multimeric display of an antigen can lead to cross-linking B cell receptors (BCRs) and an increase in the robustness of the immune response. Further it was hypothesized that disulfide-stabilized trimeric, head-only rsHAs will present the conserved RBS epitope, thereby eliciting bnAb responses. Trimerization of the head-only rsHAs is hypothesized to mask potentially immunodominant, neo-epitopes present on monomers. rsHA homotrimers and heterotrimers will present three copies of the conserved RBS for potent stimulation of naïve BCRs.

The first crystal structures of influenza HA came from HA derived from virions by bromelain cleavage. Recombinant HA (rHA) expression of full-length soluble ectodomain (FLsE) requires appending a C-terminal trimerization tag (e.g. foldon or GCN4) in place of the transmembrane segments in order to present native-like trimers present on virions. Others have engineered cysteine residues at the HA1 interface of group 2 FLsE trimers in order to “lock” the HA1 heads in place to impede the viral fusion pathway. These initial cysteine modifications were later the basis for group 1 modifications (first for H1 CA-09). Engineering these cysteine residues at the trimer interface stabilizes a trimeric form of the rHA FLsE. It was hypothesized that these modifications could be inserted into the rsHA monomeric head-only constructs described herein in Example 2A and by appending a C-terminal trimerization tag creating disulfide-stabilized, head-only trimers. The trimeric rsHAs would be better immunogens because they 1) conceal neo-epitopes that would be exposed on the head-only monomers 2) present a more native-like conformation of HA present on the virion surface 3) increase the number of copies of the immunogen which may more potently stimulate BCRs.

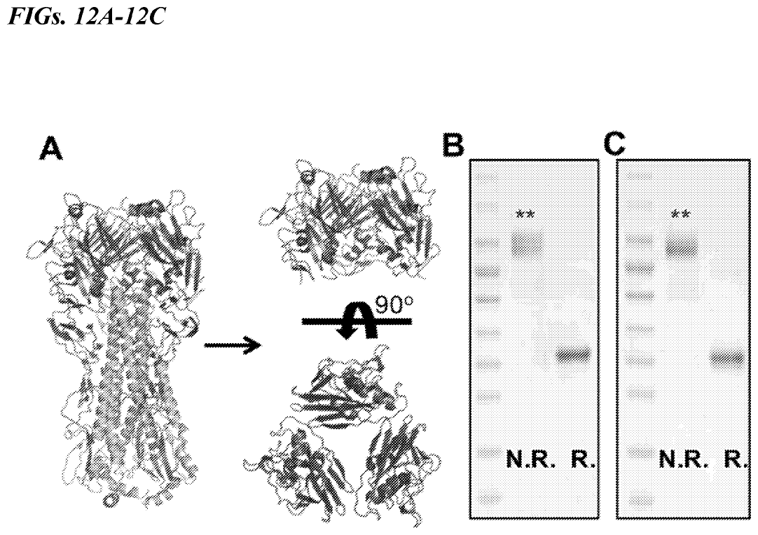

Design and characterization of cysteine-stabilized, trimeric head-only immunogens. Initial efforts to create trimeric, head-only constructs involved simply appending a C-terminal foldon tag after a short glycine-serine linker. However, as there are few, native stabilizing contacts between the head domains in the trimer (most are in the HA2 “stem”) these constructs were prone to proteolytic cleavage and subsequent dissociation into monomers. The inventors engineered cysteines at the HA1 head trimer interfaces to “lock” the heads together, a technique that was previously successful for stabilizing full-length trimeric HA. For preliminary experiments, group 2 H3 AI-68 and H14 WI-10 were tested as well as rsH14WI with the H3 AI-68 RBS. These constructs were efficiently expressed in insect cells and, after cleavage of the foldon trimerization tag, a stable trimeric species was isolated using size-exclusion chromatography; on an SDS-PAGE gel under non-reducing and reducing conditions, the isolated protein ran as a trimeric and monomeric species, respectively (FIG. 12). An important property of these constructs is the removal of the potentially immunogenic foldon and 6xHis tags. Additionally, isolating the stable trimeric species ensures that there are no neo-epitopes (present on monomers or dimers). These neo-epitopes would also not be present on the the virion surface and humoral responses directed against these epitopes would likely not offer protection. These data demonstrate the feasibility of creating cysteine-stabilized, head-only immunogens of our rsHAs.

Homotrimeric rsHAs as immunogens. It is specifically contemplated herein that cysteine-modified, rsHA constructs for the H1 and H3 RBS grafts can be pursued using the methods described herein. One goal is to identify at least three rsHA scaffolds from either group 1 or group 2 that will accept the cysteine modifications and form stable trimers. These rsHAcys-cys constructs are homotrimeric with three identical copies of each monomeric rsHA. To begin, cysteines are engineered into the wildtype, head-only HAs to ensure efficient trimerization is possible. rsHA cysteine-modification efforts can be prioritized based on the efficiency of the wildtype constructs. All constructs will be expressed in insect cells with a HRV-3C cleavable foldon and 6xHis tags. Post-cleavage the trimeric, dimeric and monomeric species will be separated by size-exclusion chromatography and analyzed by SDS-PAGE.

Heterotrimeric rsHAs as immunogens. Also contemplated herein is the design of cysteine-stabilized rsHA heterotrimers. This immunogen would present 3 redundant copies of the desired RBS epitope but would eliminate the redundancy of the non-desired epitopes in each of the monomeric scaffolds. In other words, in the homotrimers described above each component of the trimer is identical, therefore there are, in theory, three copies of any possible epitope. However, if cysteine modifications are used to create rsHA heterotrimers the ratio of the bnAb RBS epitope over unwanted epitopes can be increased: each component of the heterotrimer with only the RBS donor graft conserved between the monomers.

For optimized protein expression of this type of construct a polycistronic vector for mammalian and insect cells was generated. This vector has three unique cloning sites that have appended to the foldon trimerization sequence one of three unique purification tags: His6x (HHHHHH), strep II (WSHPQFEK), or FLAG (DYKDDDDK). A unique rsHA head-only construct with the Cys-Cys modifications can be cloned upstream of each of these tags. Each cloning site is separated by a sequence encoding the “self-cleaving” 2A peptide from porcine teschovirus-1. This expresses, from one plasmid, a heterotrimeric, rsHAcys-cys. With different C-terminal tags on each of the rsHA sequential purification steps can be performed to enrich for heterotrimeic rsHA (or “chimeras”). Because the location of the engineered cysteines are different for group 1 and group 2 influenzas it unlikely that a chimera would stably form containing both group 1 and group 2 rsHAs. The generation of chimeric rsHAcys-cys is specifically contemplated herein.

Design of nanoparticles as a platform for multimerized display of rsHAs. Oligomerization of an antigen can lead to efficient cross-linking of BCRs and an overall increase in the robustness of the immune response. In addition to the trimerization efforts described above, a self-assembling nanoparticle approach can be pursued to multimerize trimeric rsHAs to augment BCR responses. The inventors have established self-assembling nanoparticles based on the helicobacter pylori ferritin molecule reported previously using H1 HA FLsE. The ferritin-HA FLsE fusion nanoparticle displays three copies of HA on the three-fold axis, recapitulating the native-like FLsE HA trimer. On a given nanoparticle there are 24 copies of HA displayed. The ferritin-HA FLsE construct can be modified to display the 1) rsHAs and 2) chimeric rsHAcys-cys head-only constructs. Expression can be done in mammalian HEK 293T cells. After expression and purification, negative-stain electron microscopy can be performed on the nanoparticles to ensure that they have the desired properties. Then the nanoparticles can be tested for reactivity against the panel of RBS-directed Abs to ensure that the nanoparticle assembly did not affect antigenicity.

Functional characterization of multimerized immunogens using B-cell activation assays. Well-established BCR-activation assays can be used as a surrogate for the engagement by an antigen of the BCR during an immune response. This assay can be used to triage designed immunogens to find the optimal oligomeric/multimerized immunogen to use in in vivo testing. Monomeric rsHAs, homo and heterotrimeric rsHA and rsHA-decorated nanoparticles can be tested using this assay. Initially, two pairs of H1 and H3 RBS-directed Abs (Table 1) are selected as well as their inferred UCAs as surrogates of the type of memory and naïve BCRs aimed to elicit. Their VH and VL sequences can be subcloned into membrane bound-IgM expression vectors and cell-surface express. Calcium-flux and tyrosine phosphorylation of downstream proteins SLP-65 and HS1 is monitered. Monomeric rsHA heads are expected to poorly stimulate the surrogate BCRs in this assay while the homo- and heterotrimeric rsHAs and rsHA-decorated nanoparticles to robustly activate in this assay.

### Example 2C

### Testing rsHAs In Vivo in both Naïve and Immune-Experienced Contexts

It is hypothesized herein that immunizations with rsHAs, in both naïve and immune-experienced contexts, will elicit a robust, broadly-neutralizing response focused to the conserved RBS.

Current influenza vaccination does not offer life-long protection. Influenza vaccine efficacy is variable from season-to-season and only effective when the HA vaccine components closely match the circulating influenza HA strain. Accumulation of mutations in HA actively escape seasonal humoral responses. Even when effective, the elicited seasonal responses are often strain-specific and not broadly neutralizing. Conversion of strain-specific responses and/or elicitation of broadly-neutralizing responses are goals of a universal influenza vaccine. In vivo testing of designed rsHA immunogens will show a higher frequency of RBS-directed responses compared to current seasonal vaccines and will demonstrate how to effectively target a subdominant epitope.

Nojima analysis. Assessing rsHAs immunogens for RBS-targeting and their superiority over current season influenza vaccines requires large-scale, single B-cell sorting/cloning and rapid epitope identification. Murine humoral responses to H1 SI-06 FLsE were previously characterized. This permits an extensive interrogation of B-cell populations and surpasses current single B-cell sorting and high-throughput V(D)J sequencing techniques in the sheer number of Abs characterized. This technique can be used to characterize rsHA immunogens. Briefly, Nojima cultures involve isolation of germinal center (GC) B cells that are single-cell sorted into 96-well plates containing the feeder cell line from modified 40LB fibroblasts called NB-21.2D9. Through activation signals that are BCR-independent, proliferation, differentiation and secretion of IgGs into the supernatant occur. Supernatants can be readily screened, in a high throughput manner, against a panel of different rHAs for reactivity using a Luminex-based assay. The V(D)J of IgGs with interesting properties can then be sequenced for recombinant expression and further characterization (e.g. structural studies).

The RBS is subdominant in the murine model. Mice were previously immunized with H1 SI-06 FLsE, H3 AI-68 FLsE and trimeric head H3 AI-68cys-cys (see FIG. 12) and used Nojima analysis to specifically determine the frequency of RBS-directed Abs to serve as a baseline for immune-focusing strategies for rsHA immunogens. The single GC B-cells were screened in competition format with two RBS-directed Abs, CH67 for H1 and HC19 for H3 to determine frequency of RBS-directed responses. For H1 SI-06 only 1.8% (4/223), for H3 AI-68 FLsE only 2.9% (5/171) and for H3 AI-68cys-cys 0% (0/162) competed with RBS-directed Abs. It was concluded that the RBS epitope is subdominant in the murine model. From the H1 SI-06 immunization a small three-membered lineage was identified and the crystal structure of one its members, FL-10, was determined in complex if H1 SI-06. It engages the RBS through CDR L1 with the same sialic-acid like contacts as a human RBS-directed Ab Ab6639 (data not shown). These data are relevant for validating the murine model for testing rsHA immunogens. First, the subdominant nature of the RBS in the murine model gives us a large “window of improvement” for showing that immunogens can increase the frequency of RBS-directed Abs. Second, they show that murine responses can produce RBS-directed antibodies with desired properties similar to the human response.

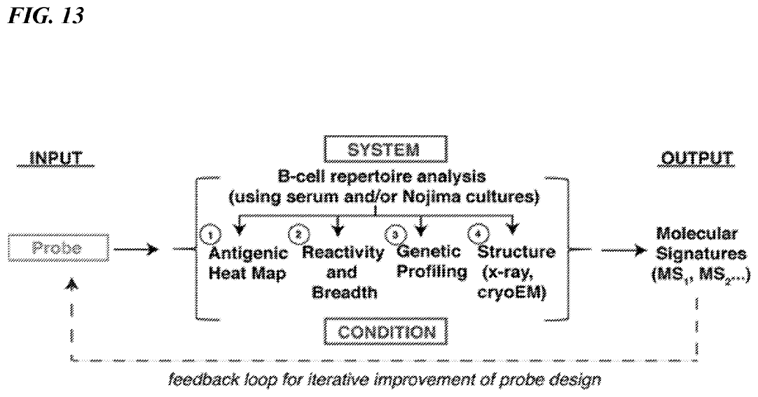

Prime-boost immunization with rsH4NBv3. To address initial concerns regarding the feasibility of this alternative approach, a first set of prime-boost experiments were performed. Mice were immunized with H1 SI-06 FLsE and 8 weeks later boosted with either homologous H1 SI-06 FLsE or with optimized rsH4NBv3 FLsE with the H1 SI-06 RBS graft (FIGS. 3A-3E). Preliminary analyses of the serum and single GC B-cell responses are shown in FIG. 13A. The data show that the heterologous boost was as efficient as the homologous boost at eliciting serum responses that directly competed with the RBS-directed bnAb CH67. This indicates that the rsHA immunogen, which only has the H1 RBS epitope in common with the initial priming immunogen, can effectively recall memory responses to the H1 RBS. Single GC B-cell analysis indicates that the homologous prime-boost produces very few B cells that interact with the rsH4NB10v3 and none that bind the wildtype H4 scaffold (FIG. 13B). In contrast, the heterologous boost with rsHA produces robust responses to the rsH4NB10v3 immunogen. H4 scaffold-specific responses were also observed. In FIG. 13C, the total number of CH67-like single GC B-cells were quantified and the data indicate an overall increase of such cells resulting from the heterologous boost with rsH4NBv3. These data show that through further characterization and subsequent optimization of vaccination strategies our immunogens can focus antibody responses to the conserved RBS.

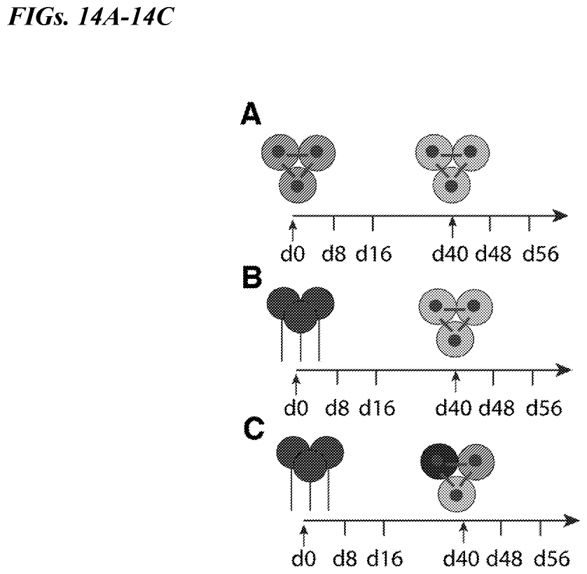

Immunizations using rsHAs. All immunizations are analyzed using Nojima cultures and the humoral responses characterized following a general workflow described in FIG. 14. This workflow is designed to explicitly assess whether the rsHA immunogens 1) boost the RBS-directed response and 2) if this response contains a higher frequency of RBS-directed bnAbs. All rsHA immunizations are compared to a standard seasonal vaccination to serve as a benchmark to calibrate rsHA immunogens and their immune-focusing properties. The use of a trivalent HA vaccine to more faithfully represent the current seasonal vaccine is specifically contemplated herein.

Mice and general immunization protocol and Nojima analysis. The human RBS-directed bnAbs require a CDR H3 of ˜18a.a. in length and a string of aromatics encoded by the JH6 segment; mice do not have an equivalent JH6 and the required CDR H3 length may be prohibitive despite having the optimized RBS-focusing immunogen. Therefore, a BL/6 mouse with a DH/JH6 knock-in was generated; the DQ52 murine allele is replaced with the human DH2˜2*01 to maximize the possible CDR H3 lengths; these mice are used for the experiments. For appropriate statistics, 11 age-matched female mice will be used per immunization. A total of 22 mice will be used for each experimental set up. Two unimmunized mice will serve as day 0 controls. 20 μg of the immunogen adjuvanted in e.g., Alhydrogel (alum) will be immunized in the hind limb. 8 and 16 days post immunization (or boost), bone marrow, lymph node and serum samples are collected in order to analyze populations of GC and memory B-cells. These populations are characterized and sorted for Nojima culture analysis by flow cytometry. GC B cells are isolated based on their characteristic phenotype. Memory B-cells (IgM+ and IgG+) are identified based on reactivity to fluorescently conjugated rHA probes and referred to as “rHA+” population; expression of IgM, IgG, CD19, CD80 and PD-L2; rHA+CD19hi B cells will be characterized by expression of PD-L2 and CD80. Initially, IgG+ memory B-cells are identified by an IgM-rHA+CD19hi phenotype and IgM+ memory B-cells, by an IgG-rHA+CD19hi phenotype. The BCRs of individual GC B and memory B-cells are characterized by clonal Nojima cultures. Processing Nojima cultures is the rate-limiting step. Approximately 40 96-well plates/experiment can be analyzed. In one experiment, the design comprises 13 plates/mouse/time with 3 mice for each sample time. The cloning efficiencies for B memory-cells and GC cells are markedly different: 70% and 20%, respectively. It is expected that from the 13 plates, 269 memory B-cell clones (4×96×0.7) are obtained and 173 GC B-cell clones (9×96×0.2) from each mouse at each sample time. As three mice will be sampled, the total expected numbers of clones recovered in a single experiment will be 807 (3×269) memory B-cell clones and 519 (3×173) GC B-cell clones for each time-point. This degree of sampling permits a thorough characterization of the humoral response post-immunization and permits quantitative definition of the efficacy of rsHA immunogens.

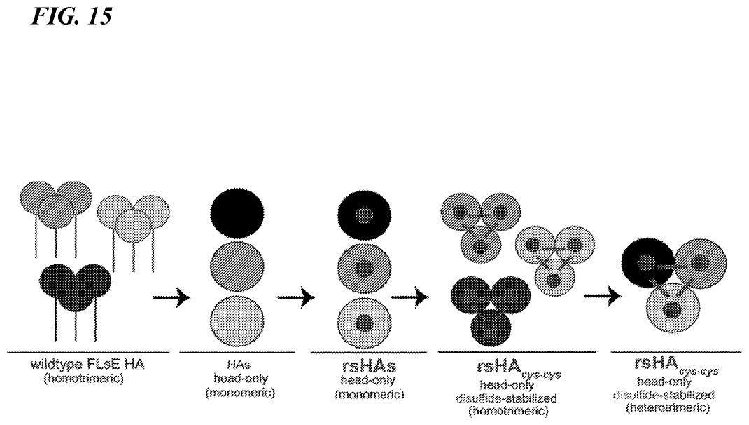

Sequential immunization with homotrimeric rsHAs to assess RBS immune focusing. Sequential immunization with homotrimeric rsHAs is assessed to determine if the prime and boost exposure of the conserved RBS donor grafts boost a broadly neutralizing, RBS-directed response. Initial experiments can start with rsHAs with the H1 SI-06 donor grafts and use two different sets of homotrimers as a prime and a boost (FIG. 15A). Day 8 and 16 GC B-cells samples are isolated and characterized as described elsewhere herein. Two different rsHA homotrimers will be used with the same donor graft in these sequential immunizations so only the conserved RBS graft is boosted. The first immunization is indicative of the naïve response to these rsHA immunogens. For comparison, this response is compared to that of immunized mice with wildtype, homotrimeric HAs for which the the donor grafts are based (e.g. H1 SI-06, H3 AI-68).

Boosting with homo- and heterotrimeric rsHAs after pre-immunzation with wildtype HA. As a surrogate for pre-existing anti-influenza humoral immunity, mice are pre-immunized with either i) H1 SI-06, ii) H3 AI-68 or iii) H7 SH-13 and then assess the response to the rsHAs. The rationale for selecting these HAs as the prime is as follows. For H1 SI-06 this directly tests whether the rsHAs with the HI-RBS donor grafts can boost and/or “refocus” the secondary response to the RBS. For H3 HK-68, despite having rather different sequences surrounding the RBS, the overall structural features of the RBS between H1 and H3s are nearly indistinguishable and there are indeed cross-reactive bnAbs. Therefore, RBS-directed Abs can be boosted, recognizing the common structural features of the RBS. For the boost, the homotrimeric rsHA was used (FIG. 13B). The heterotrimeric rsHA will also be tested to determine whether reducing the number of copies of the scaffold while maintaining three copies of the donor graft itself will increase the RBS-directed response (FIG. 15). Nojima samples are isolated and characterized as described elsewhere herein. These immunizations representing preexisting immune histories (Original Antigenic Sin) are simplified for pre-immunization with only 1 wildtype rHA. It is appreciated that this generally does not represent the multiple exposures (e.g. vaccination/infection) that a given individual likely experiences in their lifetime.

Characterization of the humoral responses post immunizations. An INPUT-OUTPUT analysis is used for immune-profiling of B-cell repertoires post-vaccination with rsHAs. This approach is optimized to rapidly characterize humoral responses to determine if immunization with the rsHAs have 1) altered patterns of immunodominance and 2) whether there is a higher frequency of bnAbs targeting the conserved RBS. There are three key variables: 1) the Probe 2) System and 3) Condition of the System. The Probe is the rsHA immunogen. The System refers to the organism (e.g. BL/6 mouse) Lastly, the Condition of the System is a variable specifically meant to address the impact of preexisting immunity and its influence on the humoral responses to the Probe.

First, “antigenic heat maps” of the humoral responses are generated. This approach epitope bins the elicited responses using a panel of previously published antibodies with known molecular footprints (determined by x-ray crystallography) that cover the entire molecular surface of HA. This binning will quantify the number of isolated antibodies and establish the patterns of epitope dominance. Second, standard ELISA, HAI and neutralization-based assays are used to define the reactivity and “breadth” of the elicited responses. Third, genetic profiling of Abs obtained from the epitope bins of our antigenic heat maps is pursued. This will determine whether there are preferred gene usages for a particular epitope, including the RBS. Finally, structures of the rsHA in complex with murine RBS-directed Abs will be obtained to compare to human bnAbs. Collectively, these analyses provide the OUTPUT and define the “molecular signature” (MS) or “blueprint” of the humoral response for each Probe. The seasonal vaccine or wildtype monovalent HAs (described above) will establish the initial baseline molecular signature (MSO). The rsHA probes will be used to obtain MS1, MS2 . . . MSn. Each MS serves as a comparator in evaluating design goals of focusing to the conserved RBS and which probe was or was not effective in doing so. This analysis platform has a “feedback loop” for iterative improvement and further probe optimization.

Characterization of serum and single B-cell responses using neutralization and HAI. The serum response can be characterized by 1) neutralization and 2) hemagglutination inhibition (HAI). The current gold-standard for measuring influenza vaccine efficacy is the hemagglutination inhibition (HAI) assay. This assay tests post-vaccination sera and its ability to effectively block influenza virus interactions with sialic acid on the surface of red blood cells (RBCs). A lower-threshold of 40 for HAI titres is set, consistent with the CDC guidelines for 50% protection. Authentic viruses are used when possible and retroviral pseudotyping of the HA when biosafety levels do not permit use of authentic virus. The inventors will use for group 1, H1 SI-06, H1 CA-09 and for group 2 H3 AI-68 and H3 HK-14 as authentic viruses and pseudotype BSL3 viruses (e.g. H4 NB-10 and H14 WI-10) to test serum neutralization titres and HAI activity. Resources (e.g. virus strains) can be obtained from BEI. It is expected that the rsHAcys-cys immunogens will increase serum reactivity to group 1 and group 2 rHAs with higher HAI and neutralization EC50s compared to wildtype HAs. This will be a direct consequence of immune focusing to the conserved RBS.

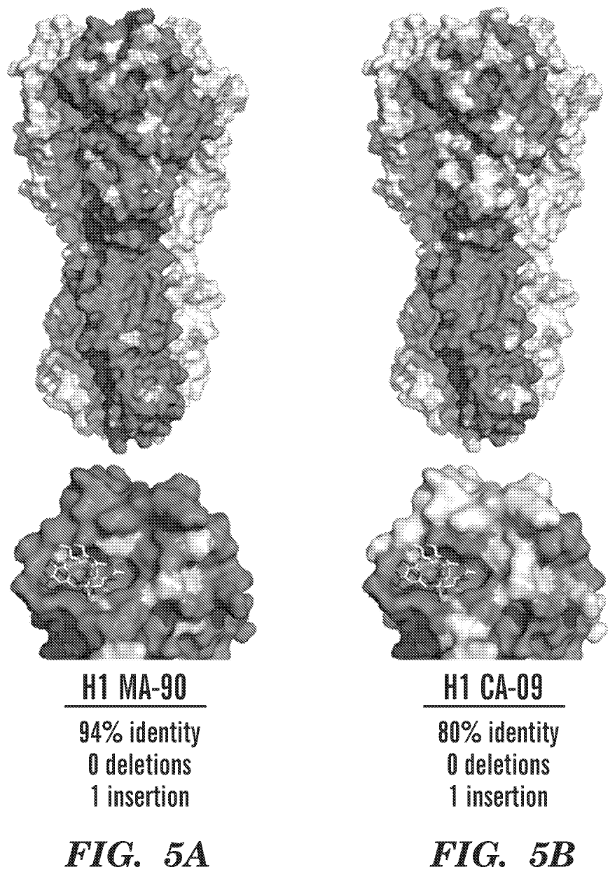

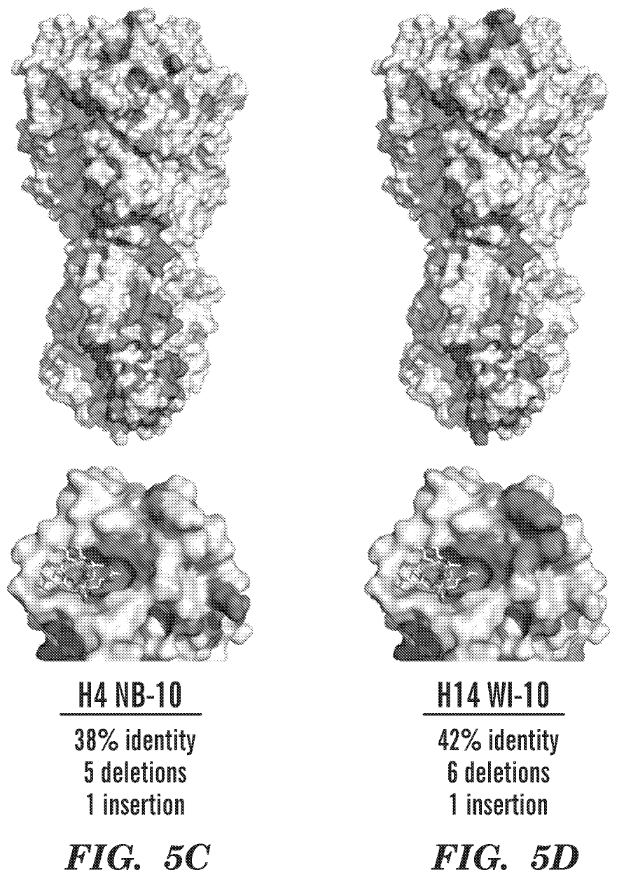

Reactivity of serum and single B-cell cultures with a diverse panel of HAs. Serum and single B-cell culture supernatants are screened to quantify reactivity to the components of the homo- and heterotrimers and to a selected panel of diverse HAs from group 1 and 2. FLsE rHAs from nearly every subtype in FIG. 5D are used—which will allow for a comprehensive comparison of reactivity. Serum is tested in an ELISA-based assay and the Nojima culture supernatants are screened, and clonal IgG specificity and avidity is determined using a Luminex-based assay, as described. The reactivity data is quantified based on avidity indices and create a “reactivity heat-map” for visual and statistical comparison. It is expected that rsHA immunogens will elicit broadly reactive RBS-directed Abs.

Isolation and characterization of RBS-directed bNAbs. The inventors will additionally screen, in competition format, with CH67 (or another RBS-directed antibody from the assembled Ab panel (FIGS. 7A-7C)) the Nojima single B-cell supernatants. For those Abs that were identified in the phenotypic screening, the V(D)J sequence will be amplified for recombinant protein expression and further characterization (e.g. structure determination). In particular, this can help identify additional Abs for comparison with the known human RBS-directed Abs for which the resurfaced proteins are based upon. It is expected that the murine humoral response to the RBS will be increased after immunization with our rsHA immunogens.

### Example 3

### Introduction

Influenza evolves primarily at the human population level and within its animal reservoirs (swine and avian) (1). In response to host humoral pressure, which predominantly targets the viral hemagglutinin (HA), the virus mutates, rendering previous immune responses suboptimal. The humoral response then evolves, through immune memory and B cell affinity maturation (2-5). When stimulated by a new exposure (infection or vaccination), memory cells can undergo new rounds of somatic hypermutation and selection. Mutated HA with reduced affinity for a particular antibody can in principle select for mutations in the latter that restore strong binding. The net effect of this on-going selection across the entire population exposed to the virus is a virus-immunity “arms race”.

Seasonal vaccination is still the most effective protection against influenza and it can compensate for “antigenic drift” but it requires predicting the newly drifted, circulating strains within the human population. The annual revision of vaccine components is often, however, suboptimal in this matching (6). A “universal” influenza vaccine should protect against seasonal variation (drift) and against introduction of new subtypes (shift) (7-10). This broad protection will likely come from a humoral response to conserved sites on the viral HA (11). The two relatively invariant epitopes so far recognized are the receptor binding site (RBS) on the HA “head” and a surface along the HA “stem” (12). These are sites of vulnerability because the virus cannot readily mutate within these regions without comprising viral fitness.

Complicating introduction of a universal influenza vaccine is how to overcome preexisting immunity present in the human population. The initial influenza exposure shapes subsequent immune responses through imprinting (13-18). This immunological memory, or “Original Antigenic Sin”, was first described by Francis and colleagues in the 1950s (19, 20). Memory B cells from previous influenza exposures or repeated seasonal vaccinations are readily re-activated to undergo additional rounds of somatic hypermutation and affinity refinement. This continual updating of memory responses leads to refined, strain-specific responses often at the expense of broadly neutralizing antibodies (bnAbs). The development of a universal influenza vaccine requires selecting an immunogen(s) that can redirect preexisting humoral responses.

Considerable effort has centered on stem-directed immunogens to elicit bnAbs while little effort, if any, has explored the RBS as a candidate immunogen for a universal influenza vaccine. The RBS is a complex, conformationally-specific epitope involving multiple segments, separated in linear sequence, but adjacent in conformational space (21). The “core” RBS residues are conserved across HA subtypes and are optimized to engage the cellular receptor sialic acid (SA). Variation within the core cannot readily occur without compromising viral fitness. Immunogen(s) designed to elicit Abs targeting this complex epitope must present it in the correct conformation.

The use of Nature's own molecular scaffolds of non-circulating influenza HAs to graft the H1 RBS core is described herein. These “natural” protein scaffolds circumvent the significant challenge in designing a molecular scaffold de novo that correctly presents the conformationally-specific and complex RBS. These “acceptor”, resurfaced HA (rsHA) scaffolds present the “donor” conserved, RBS core from circulating Hls and also remove epitopes targeted by strain-specific responses in immune-experienced individuals. The crystal structure of a rsHA in complex with a broadly neutralizing antibody (bnAb) was determined. Through structure-guided optimization, two antigenically distinct molecular scaffolds to bind a diverse panel of pan-H1 and H1/H3 cross-reactive bnAbs targeting the RBS were improved. This approach focuses the immune response to the broadly neutralizing RBS epitope and overcomes preexisting immunity present in the population. Strategies described herein provide novel candidate immunogens for a universal flu vaccine, by exploiting the immunogenicity of the conserved RBS.

Grafting the H1 SI-06 RBS onto Acceptor HA Scaffolds.

RBS from circulating H1 influenzas was used as the basis of “donor” graft to scaffold its RBS core onto antigenically distinct HA subtypes not circulating in the population (FIG. 1A). The H1 RBS core can be grouped into roughly three antigenic clusters from 1977-1994, 1995-2008 and 2009-present; prototypical members of each included H1 Massachusetts/1/1990 (H1 MA-90), H1 Solomon Islands/3/06 (H1 SI-06) and H1 California/04/2009 (H1 CA-09) (FIGS. 1B and 4) (13). The first two clusters are most notably distinguished by the loss of residue K133a in strains post 1995; a major shift occurred with the new pandemic, H1 CA-09 and has remained nearly invariant since. Despite some H1 RBS-core variation, bnAb can span these antigenic clusters (e.g. CH67 (22), 641-1-9 (9), Ab6639 (23), 5J8 (24)). The four “segments” of the RBS core were defined as S1-S4 (FIG. 1B) from H1 SI-06 for grafting. These segments include 7 of the 13 critical SA-contacting residues (FIG. 4). Many of the remaining residues (e.g. Y95 and W153) not included in the graft are in the base of the RBS and are conserved/nearly invariant across influenza viruses.

For the initial acceptor HA scaffolds, two non-circulating group 1 influenzas: H6N8 A/widgeon/Wisconsin/617/1983, H16N3 A/laughing-gull/Delaware Bay/296/1998 and two group 2 influenzas: H4N6 A/America black duck/New Brunswick/00464/2010 and H14N6 A/mallard/Wisconsin/100S3941/2010 were selected. These non-circulating HAs have little sequence conservation in reference to H1 SI-06 (FIG. 5). The acceptor HA S1-S4 boundaries were defined by aligning the H1 SI-06 sequence (FIG. 1C). The resulting resurfaced HAs (rsHAs) have the following nomenclature: “rsH4NBvX”; the resurfaced (rs) hemagglutinin scaffold subtype (H4), with an abbreviated strain name (NB) and different versions (vX).

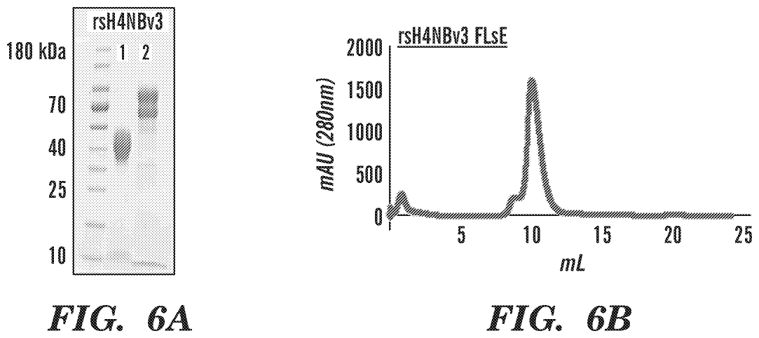

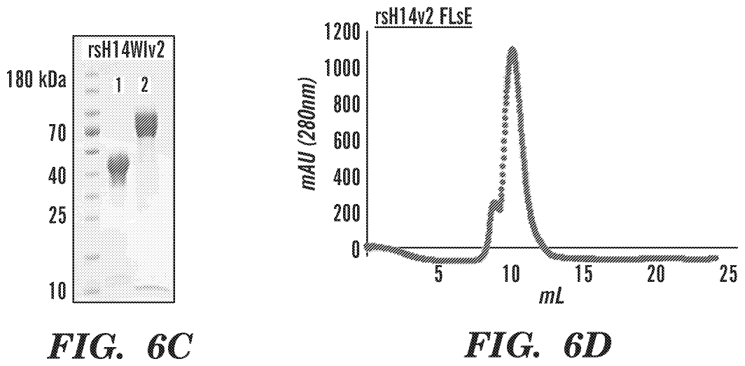

To assay for successful grafting it was determined if the proteins could be overexpressed in mammalian and/or insect cells. Both monomeric rsHA “heads” (HA1 residues 52-267 and residues 37-319 for group 1 and 2, respectively) and full-length soluble ectodomains (FLsE), were designed as previously described (9, 25). The intra-group grafting of the H1 SI-06 RBS onto the H6 or H16 scaffolds was not successful. However, as head and FLsE proteins, the inter-group transfers onto the H4 and H14 scaffolds could be overexpressed (FIG. 6) resulting in the first generation rsH4NBv1 and rsH14WIv1 constructs.

Binding Affinities of rsHAs with bnAbs

It was next determined whether the rsH4NBv1 and rsH14WIv1 constructs correctly presented the H1 RBS core by obtaining binding affinities using a panel of RBS-directed antibodies by biolayer interferometry (BLI). This panel included six H1 and/or H1/H3 cross-reactive bnAbs that engage the RBS through receptor mimicry and/or have receptor-like contacts (8, 9, 24-26). Each antibody has a footprint that overlaps with conserved contacts made by sialic acid but has different angles of approach and different peripheral contacts comprising the antigen-combining site (FIG. 7). As seen in Table 1, neither construct bound the entire panel of RBS-directed Abs. rsH4NBv1 bound only the H1/H3 cross-reactive K03.12 with an equilibrium dissociation constant (KD) ˜5.2× greater than wildtype H1 SI-06. rsH14WIv 1 bound H1 bnAb CH67 and both H1/H3 cross-reactive Abs K03.12, C05 with KDs ˜17×, ˜2.7× and ˜1.3× greater than wildtype H1 SI-06. None of the antibodies bound wildtype H4 NB-10 or H14 WI-10 the scaffolds used for the grafting experiments. These data indicate that either 1) there are peripheral residues in the scaffolds that are impeding binding and/or 2) the conformation of the RBS graft is being presented in an altered conformation.

TABLE 1
Affinity measurements of rsHAs to a panel of RBS-directed Abs. KDs (in +μM) were obtained by
applying a 1:1 binding isotherm using vendor-supplied software with at least three independent
concentrations. The VH gene usages are listed for each antibody. KDs beyond the limit of detection are
reported as >100 μM. Values are for monomeric HA heads and Fabs.

| | CH67 | 641 I-9 | H2526 | H2227 | K03.12 | C05 |
|---|---|---|---|---|---|---|
| | V_{H}1 ~ 2 | V_{H}4 ~ 59 | V_{H}1 ~ 69 | V_{H}4 ~ 4 | V_{H}1 ~ 2 | V_{H}3 ~ 23 |
| H1 SI-06 | 0.57 | 0.67 | 0.56 | 0.39 | 0.83 | 1.1 |
| H4 NB-10 | >100 | >100 | >100 | >100 | >100 | >100 |
| H14 WI-10 | >100 | >100 | >100 | >100 | >100 | >100 |
| rsH4NBv1 | >100 | >100 | >100 | >100 | 4.3 | >100 |
| rsH4NBv3 | 1.6 | 17.7 | 0.93 | 0.67 | 0.65 | 0.34 |
| rsH14WIv1 | 9.5 | >100 | >100 | >100 | 2.2 | 1.4 |
| rsH14WIv2 | 0.31 | 2.7 | 0.20 | 1.0 | 1.1 | 0.065 |

TABLE 2
Crystallographic Data Collection and Model Refinement statistics.
Statistics for the highest-resolution shell are shown in parentheses.
Data collection and processing

| | |
|---|---|
| Wavelength (Å) | 0.999 |
| Resolution range (Å) | 46.05-4.0 |
| Space group | C 1 2 1 |
| Unit cell a, b, c (Å) | 92.3, 141.5, 166.9 |
| α, β, γ (°) | 90, 102.6, 90 |
| Total reflections | 63073 (4479) |
| Unique reflections | 17320 (1493) |
| Multiplicity | 3.6 (3.0) |
| Completeness (%) | 97.5 (85.8) |
| Mean I/sigma (I) | 4.51 (1.52) |
| R_{merge} | 0.29 (0.73) |
| Rₘₑₐₛ | 0.34 (0.89) |
| Rₚᵢₘ | 0.17 (0.49) |
| CC1/2 | 0.95 (0.54) |
| CC* | 0.99 (0.84) |

Refinement

| | |
|---|---|
| R_{work}/R_{free} | 0.29/0.32 |
| RMS bonds (Å)/angles (°) | 0.003/0.70 |
| Ramachandran favored/outliers (%) | 94.65/0.14 |
| Average B-factor | 97 |

Structure of rsH4NBv1 in Complex with bnAb, K03.12.

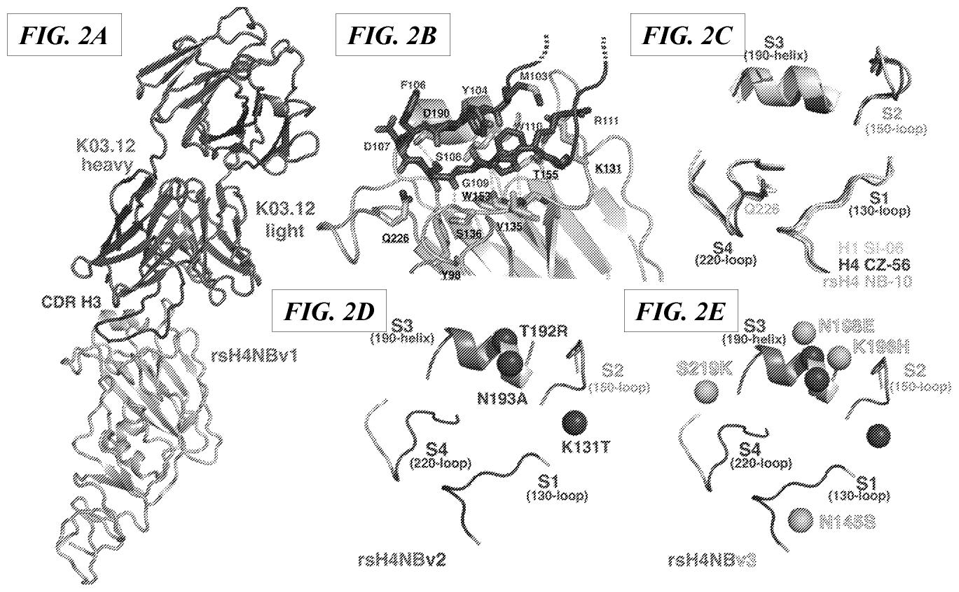

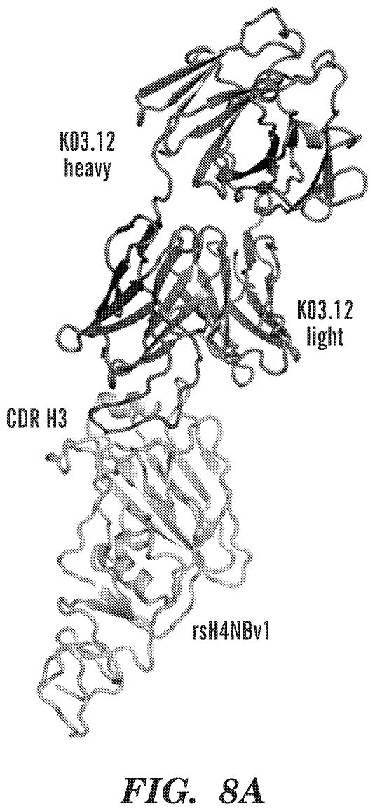

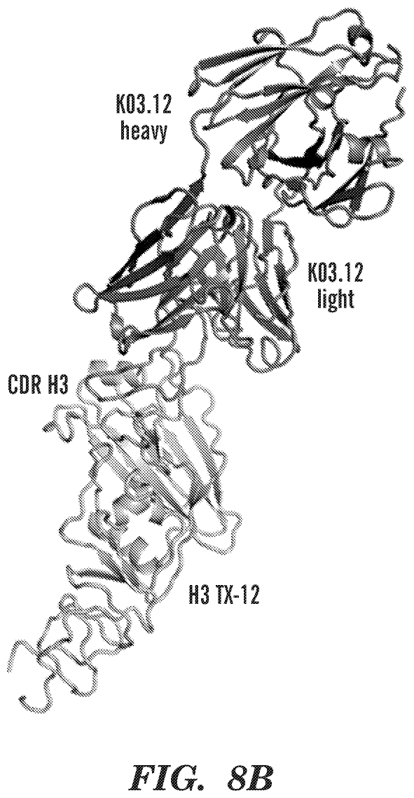

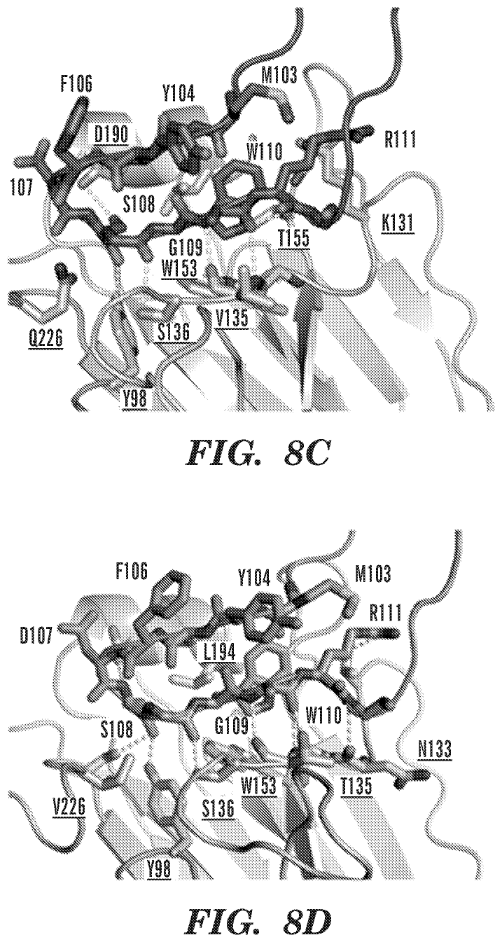

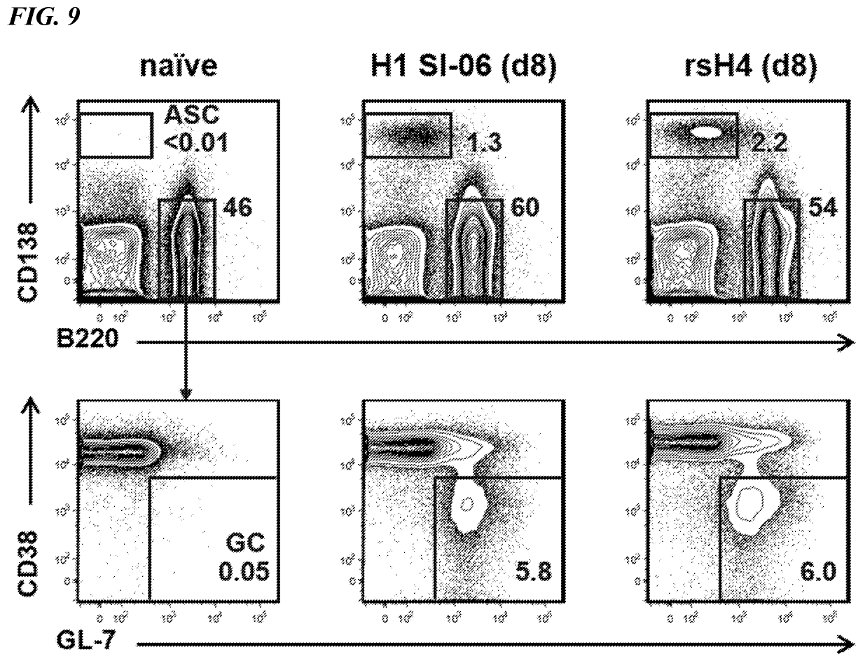

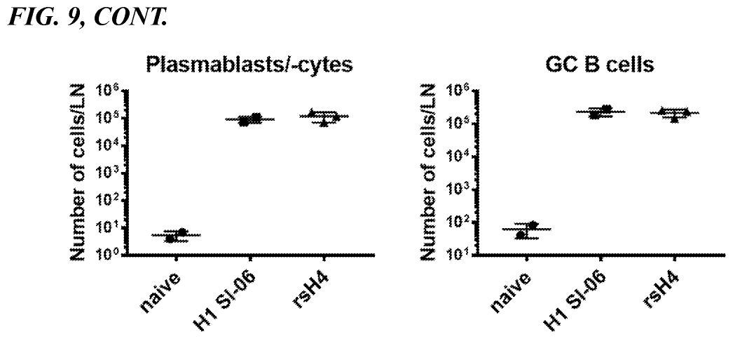

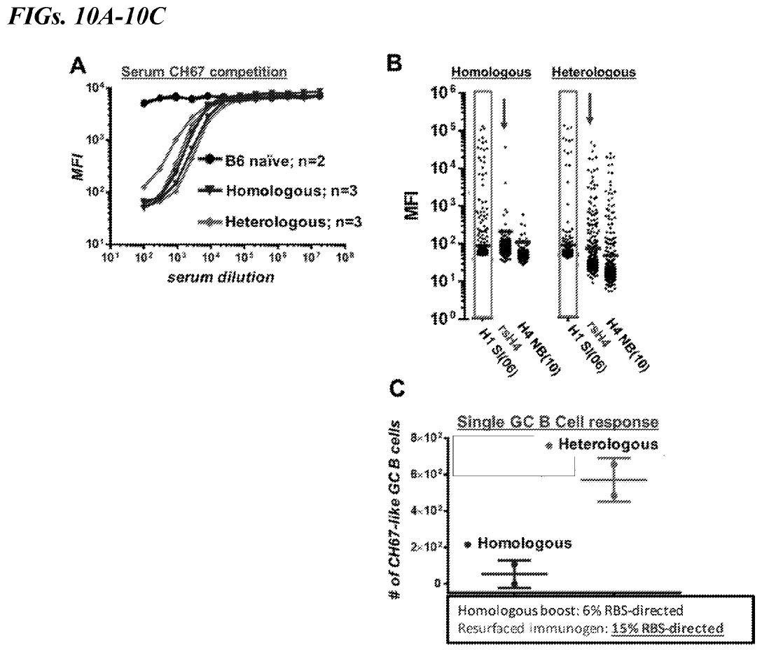

The crystal structure of the rsH4NBv1 “head” in complex with Fab K03.12 was determined (FIG. 2A). Consistent with previous characterization, C05 antibody, K03.12 engages the RBS with almost exclusively CDR H3-dependent contacts (8). The antibody contacts 14 of the 26 residues making up the four segments of the RBS graft. Additional contacts are made with conserved residues critical for SA-interactions at the base of the RBS including Y95, W153, T155 and H183. Comparison of the K03.12-rsH4NBv1 structure and the K03.12-H3 Texas/50/2012 complex shows nearly identical approach with a slight twist and rocking of the VH: VL towards HA about the principle axis (FIGS. 8A and 8B) (26). The contacting residues within the antigen-combining sites between the two structures are nearly identical (FIGS. 8C and 8D). Overall comparison of the rsH4NBv1 HA to a wildtype H4 A/duck/Czechoslovakia/1956 (PDB 5XL3) and wildtype H1 A/Solomon Islands/3/2006 (PDB 5UGY) shows no significant RMSD in S2-S4 of the RBS graft (FIG. 2C). There is, however, a displacement about S1 (150-loop) with a shift of ˜3 Å using residue N158 as reference (FIG. 2C). The overall conformation of the grafted RBS appears to be similar to that of the RBS from a wildtype HA.

Structure-Guided Improvement of the rsHA Scaffolds.

To improve the rsHA scaffold for binding to the panel of RBS-directed bnAbs (Table 1), the respective Fabs were docked onto the rsH4NBv1 structure to identify residues within the scaffold that may be modified to either 1) alleviate steric clashes and/or 2) reinforce interactions. rsHA scaffold residues were mutated to the corresponding H1 SI-06 HA. Two additional versions of the rsH4NBv1 scaffold were designed. For rsH4NBv2, three mutations were made: K131T, preceding the S1 segment and T192R and N193A, both adjacent to the S3 segment (FIG. 2D). For rsH4NBv3, four additional mutations were made, N145S, K196H, N198E, and S219K, all in the RBS periphery (FIG. 2E). No changes were made to the original segments. Scaffold improvement was initially assayed in an enzyme-linked immunosorbent assay (ELISA) (FIG. 3). None of the antibodies bound to wildtype H4 NB-10 (FIG. 3A). The second generation scaffold, v2, increased affinity, relative to v1, for four of the five antibodies (FIGS. 3B and 3C). Finally, rsH4NBv3 had high affinity-binding to all five RBS-directed antibodies (FIG. 3D). Based on the optimized rsH4NBv3 construct, it was determined whether the same seven mutations defining this scaffold could be made in the context of the rsH14WIv 1 scaffold to increase its affinity for the panel of RBS-directed antibodies. The following six mutations were made: N145S, T192R, S193A, K196H, A198E and S219K; a threonine was already present at position 131. The resulting rsH14WIv2 construct conferred binding to the entire panel of RBS-directed antibodies (FIGS. 3G-3I). For reference, H1 SI-06 reactivity to the RBS-directed panel is shown (FIG. 3I). A summary of the KDs are shown in FIGS. 3E and 3I.

The optimized scaffolds in were tested BLI to obtain more accurate KDs, free of avidity affects present when using the IgG and trimeric FLsE constructs present in ELISA. As seen in Table 1, both optimized scaffolds significantly increased affinities over their first-generation counterparts; rsHAs based on both optimized scaffolds bound all six bnAbs included in the panel. In particular, the rsH14WIv2 scaffold yielded affinities for CH67, H2526 and C05 even greater than those of wildtype H1 SI-06. In some cases, (e.g. C05) the optimized resurfaced scaffold bound the Fab >10-fold more tightly than did wildtype H1 SI-06. In general, the rsH14WIv2 had affinities closest to the wildtype H1 SI-06. These data indicated a set of key residues, in addition to the initial RBS-donor grafts, that can be grafted onto other potential scaffolds to present an “optimized” epitope to bind (or elicit) a diverse set of RBS-directed bnAbs.

The data described herein show that molecular grafting of a complex epitope recognized by broadly neutralizing antibodies is a successful strategy. The H1 SI-06 RBS core was successfully grafted onto two antigenically distinct HA scaffolds. Non-circulating HAs were used as scaffolds specifically to exploit the overall architecture of the HA protein; HA is evolutionarily optimized to adopt a similar fold within the RBS core in order to engage either a2,3 (avian receptor) or a2,6 sialic acid (human receptor). This approach circumvented the significant challenge in de novo scaffold design for presenting the conformationally specific and complex epitope of the RBS. While computational design of novel protein scaffolds has been done for HIV and RSV (27), the grafted epitopes were often less-complex (e.g. a single alpha-helix (28)) than the influenza RBS. It was surprising that both of our intra-subtype grafting failed (e.g. H6, H16), while the inter-subtype scaffolds (e.g. H4, H14) succeeded. Without wishing to be bound by a particular theory, one possible explanation is that the group 2 cluster of “acceptor” scaffolds includes the only other circulating influenza in the human population, the H3 subtype. The data indicate that other non-circulating HA scaffolds should focus on the group 2 influenzas.

The rsHA scaffolds described herein are based on HA subtypes, that, to date, have never circulated in the human population and therefore are considered “immune-naïve”; they would not boost strain-specific responses in immune-experienced individuals. Through structure-guided improvement, the optimized rsH4 and rsH14 scaffolds bind with high-affinity to a diverse panel of pan-H1 and H1/H3 cross-reactive, RBS-directed bnAbs. Importantly, these Abs represent the type of response that may be required to elicit by a universal influenza vaccine. Collectively, the Abs in the panel (Table 1) can bind all H1 isolates both pre-pandemic (<2009) and post-pandemic (>2009) as well as circulating H3 influenzas. These rsHAs immunogens can elicit responses that protect against both circulating H1 and H3 influenzas.

Current influenza research has focused on the development of a universal influenza vaccine. Such a vaccine should induce broad immunity a) within seasonal, circulating H1 and H3 subtypes, b) across subtypes (heterosubtypic) and c) pre-pandemic (e.g. H5, H7). The pathway to achieving these criteria will likely come from eliciting humoral responses to conserved sites on HA such as the RBS and “stem” (8). Significant effort has focused on targeting the latter through the development of immunogens that either selectively display the HA2-stem (29) or chimeric HAs that present circulating H1, H3 stems with a heterologous HAI-head (12). While promising, both of these approaches ignore 1) the invaluable contribution of RBS-directed bnAbs in preventing infection and 2) the primarily ADCC-dependent mechanism of action of stem-directed bnAbs, which will only contribute by arresting the spread of an already established infection. It is likely, however, that a universal vaccine will necessitate incorporation of both bnAbs targeting the conserved RBS and stem epitopes.

An additional hurdle that must be overcome on the pathway to a universal vaccine is addressing preexisting immunity present in the human population. The stem-directed approaches described above rely on either completely removing or masking the antigenicity of the highly variable HAI head, against which most of the preexisting strain-specific responses are directed. The “molecular surgery” performed here accomplishes a similar objective. The “acceptor” HA scaffolds will have little or no preexisting immunity within the human population. The candidate immunogens will redirect humoral responses in immune-experienced individuals to the conserved RBS core. The heterologous periphery of the scaffolds surrounding the RBS donor graft will reinforce humoral responses to refine conserved, RBS core contacts while adapting and accommodating foreign peripheries. Importantly, the panel of RBS-directed antibodies used to characterize the immunogens are precisely the types of antibodies that a universal vaccine should elicit. In such a polyclonal response, the different angles of approach and peripheral contacts indicate that development of resistance to a collection of bnAbs focused on the RBS would be a significant hurdle. This epitope scaffolding approach described herein will serve as a vaccine platform for other rapidly evolving pathogens for which preexisting immunity is present (e.g. RSV and dengue). This refocusing strategy would both redirect and elicit bnAbs in the human population.

### Materials and Methods

### Expression and Purification of HA

rHA1 and “head” and rHA full length soluble ectodomains (FLsE) constructs were cloned into pFastBac vector for insect cell expression (Hi5 cells) or pVRC vector for mammalian expression (293F or 293T cells). HAs were derived from the following templates: H4N6 A/America black duck/New Brunswick/00464/2010 (GenBank: AGG81749.1), H6N8 A/widgeon/Wisconsin/617/1983 (GenBank: AHM99985.1), H14N6 A/mallard/Wisconsin/100S3941/2010 (GenBank: AGE03043) and H16N3 A/laughing-gull/Delaware Bay/296/1998 (GenBank: AFX85524.1). All constructs were confirmed by DNA sequencing at the DNA Sequencing Core Facility at Dana Farber Cancer Institute. For biolayer interferometry (BLI) and crystallography the HA1 head constructs contained a HRV 3C-cleavable C-terminal His6X tag or SBP-His8Xtag. The HA FLsE constructs used in ELISA assays contained a thrombin or HRV 3C-cleavable C-terminal fold on tag with either a His6X or SBP-His8Xtag. All constructs were purified from supernatants by passage over Cobalt-TALON resin (Takara) followed by gel filtration chromatography on Superdex 200 Increase (GE Healthcare) in 10 mM Tris-HC1, 150 mM NaCl at pH 7.5. For BLI and crystallography the tags were removed using HRV 3C protease (ThermoScientific) and the protein repurified using Cobalt-TALON resin to remove the protease, tag and non-cleaved protein.

### Fab and IgG Expression and Purification

For Fab and IgG production the genes for the heavy- and light-chain, variable domains were synthesized and codon optimized by Integrated DNA Technologies and subcloned into pVRC protein expression vectors containing human heavy- and light-chain constant domains, as previously described (9, 18). Heavy-chain constructs for Fab production contained a non-cleavable His6X tag; for IgG heavy constructs there was no cleavable purification tag. Constructs were confirmed by sequencing at the DNA Sequencing Core Facility at Dana Farber Cancer Institute. Fabs and IgGs were produced by transient transfection in suspension 293F or adherent HEK 293T cells using Lipofectamine 2000 (Invitrogen) or polyethylenamine (PEI). Supernatants were harvested 4-5 days later, clarified by centrifugation. Fabs were purified using Cobalt-TALON resin (Takara) followed by gel filtration chromatography on Superdex 200 Increase (GE Healthcare) in 10 mM Tris-HCl, 150 mM NaCl at pH 7.5. IgGs were purified using Protein G Plus Agarose (ThermoFisher Scientific). Briefly, IgG supernatants were incubated overnight with agarose slurry, eluted with 0.1M glycine, pH 2.5 and normalized with 1M Tris-HCl, pH 8.0 and dialyzed against PBS buffer overnight.

### Crystallization and Data Collection.

rsH4NBv1 HA1 head domain and K03.12 Fab were incubated at 1:1.5 molar ratio, respectively. The complex was isolated by size exclusion chromatography using a 24 mL Superdex Increase equilibrated in 10 mM Tris-HCl, 150 mM NaCl. Crystallization was achieved by hanging drop vapor diffusion at 18° C. Crystals were grown in 100 mM sodium citrate (pH 4.5), 20% (wt/vol) PEG 4000. Crystals were cryoprotected in mother liquor supplemented with 25% (vol/vol) glycerol and flash-frozen in liquid nitrogen. Data were collected at 0.999 Å with a rotation of 1° per image on the 8.2.2 beamline, Advanced Light Source, at Berkley National Laboratory.

### Structure Determination and Analysis.

The structure was determined by molecular replacement using PHASER (30, 31) with the K03.12-A/Texas/50/2012 (H3N2)-head complex (PDB ID 5W08) as a search model (ref). Density-modified, NCS-averaged electron density maps were generated with DM (CCP4) and were used as guide for model building. Refinement of individual and group B factors was performed using PHENIX (26). Model building was done in COOT (32) and assessed with MolProbity (33). N-linked glycan stereochemistry restraints were generated with Privateer (34). Figures were generated using PyMOL Molecular Graphics System (v1.8.0.0; Schrodinger LLC).

### Interferometry Binding Experiments

Interferometry experiments were performed using a BLItz instrument (forteBIO, Pall Corporation). Fab were immobilized on a Ni-NTA biosensor and cleaved rHA heads were titrated to obtain binding affinities. Initial, single-hit concentrations, were tested at 35 μM for binding and then subsequent titrations for at least three different concentrations (chosen depending on the apparent KD from the high concentration); the refined KD was obtained through global fit of the titration curves by applying a 1:1 binding isotherm using vendor-supplied software. All experiments were performed in 10 mM Tris-HCl, 150 mM NaCl at pH 7.5 and at room temperature.

### ELISA

5-10 ng of rHA FLsE were adhered to high-capacity binding, 96 well-plates (Corning) overnight in PBS. Plates were blocked with non-fat dried milk in PBS containing Tween-20 (PBS-T) for 1 hr at room temperature (RT). Blocking solution was discarded and 10-fold dilutions of RBS-directed IgGs in PBS were added to wells and incubated for 1 hr at RT. Plates were then washed three times with PBS-T. Secondary, anti-human IgG-HRP (Abcam), in PBS-T was added to each and incubated for 1 hr at RT. Plates were then washed three times with PBS-T. Plates were developed using 1-Step ABTS substrate (ThermoFisher) and immediately read using a plate reader at 410 nm. Data were plotted using Prisim 6 (GraphPad Software) and affinities determined.

### REFERENCES

1. Taubenberger J K & Morens D M (2010) Influenza: the once and future pandemic. Public Health Rep 125 Suppl 3:16-26.

2. Dal Porto J M, Haberman A M, Shlomchik M J, & Kelsoe G (1998) Antigen drives very low affinity B cells to become plasmacytes and enter germinal centers. J Immunol 161(10):5373-5381.

3. De Silva N S & Klein U (2015) Dynamics of B cells in germinal centres. Nature reviews. Immunology 15(3):137-148.

4. Foote J & Milstein C (1991) Kinetic maturation of an immune response. Nature 352(6335):530-532.

5. Victora G D & Nussenzweig M C (2012) Germinal centers. Annual review of immunology 30:429-457.

6. Dormitzer P R, et al. (2011) Influenza vaccine immunology. Immunological reviews 239(1):167-177.

7. Ekiert D C, et al. (2009) Antibody recognition of a highly conserved influenza virus epitope. Science 324(5924):246-251.

8. Ekiert D C, et al. (2012) Cross-neutralization of influenza A viruses mediated by a single antibody loop. Nature 489(7417):526-532.

9. Schmidt A G, et al. (2015) Viral receptor-binding site antibodies with diverse germline origins. Cell 161(5):1026-1034.

10. Sui J, et al. (2009) Structural and functional bases for broad-spectrum neutralization of avian and human influenza A viruses. Nature structural & molecular biology 16(3):265-273.

11. Corti D & Lanzavecchia A (2013) Broadly neutralizing antiviral antibodies. Annual review of immunology 31:705-742.

12. Krammer F & Palese P (2015) Advances in the development of influenza virus vaccines. Nature reviews. Drug discovery 14(3):167-182.

13. Bedford T, et al. (2014) Integrating influenza antigenic dynamics with molecular evolution. eLife 3:e01914.

14. Fonville J M, et al. (2014) Antibody landscapes after influenza virus infection or vaccination. Science 346(6212):996-1000.

15. Hensley S E (2014) Challenges of selecting seasonal influenza vaccine strains for humans with diverse pre-exposure histories. Current opinion in virology 8:85-89.

16. Li Y, et al. (2013) Immune history shapes specificity of pandemic H1N1 influenza antibody responses. The Journal of experimental medicine 210(8):1493-1500.

17. Moody M A, et al. (2011) H3N2 influenza infection elicits more cross-reactive and less clonally expanded anti-hemagglutinin antibodies than influenza vaccination. PloS one 6(10):e25797.

18. Schmidt A G, et al. (2015) Immunogenic Stimulus for Germline Precursors of Antibodies that Engage the Influenza Hemagglutinin Receptor-Binding Site. Cell reports 13(12):2842-2850.

19. Davenport F M, Hennessy A V, & Francis T, Jr. (1953) Epidemiologic and immunologic significance of age distribution of antibody to antigenic variants of influenza virus. The Journal of experimental medicine 98(6):641-656.

20. Jensen K E, Davenport F M, Hennessy A V, & Francis T, Jr. (1956) Characterization of influenza antibodies by serum absorption. The Journal of experimental medicine 104(2):199-209.

21. Skehel J J & Wiley D C (2000) Receptor binding and membrane fusion in virus entry: the influenza hemagglutinin. Annual review of biochemistry 69:531-569.

22. Schmidt A G, et al. (2013) Preconfiguration of the antigen-binding site during affinity maturation of a broadly neutralizing influenza virus antibody. Proceedings of the National Academy of Sciences of the United States of America 110(1):264-269.

23. Raymond D D, et al. (2016) Influenza immunization elicits antibodies specific for an egg-adapted vaccine strain. Nature medicine 22(12):1465-1469.

24. Hong M, et al. (2013) Antibody recognition of the pandemic H1N1 Influenza virus hemagglutinin receptor binding site. Journal of virology 87(22):12471-12480.

25. Whittle J R, et al. (2011) Broadly neutralizing human antibody that recognizes the receptor-binding pocket of influenza virus hemagglutinin. Proceedings of the National Academy of Sciences of the United States of America 108(34):14216-14221.

26. McCarthy K R W, A.; Kuraoka, M; Do, K. T.; McGee, C. E.; Sempowski, G. D.; Schmidt, A. G.; Kelsoe, G.; Harrison, S. C. (2017) Human memory B cells that cross-react with group 1 and group 2 influenza A viruses. submitted.

27. Correia B E, et al. (2014) Proof of principle for epitope-focused vaccine design. Nature 507(7491):201-206.

28. Ofek G, et al. (2010) Elicitation of structure-specific antibodies by epitope scaffolds. Proceedings of the National Academy of Sciences of the United States of America 107(42):17880-17887.

29. Yassine H M, et al. (2015) Hemagglutinin-stem nanoparticles generate heterosubtypic influenza protection. Nature medicine 21(9):1065-1070.

30. McCoy A J (2007) Solving structures of protein complexes by molecular replacement with Phaser. Acta crystallographica. Section D, Biological crystallography 63(Pt 1):32-41.

31. McCoy A J, et al. (2007) Phaser crystallographic software. Journal of applied crystallography 40(Pt 4):658-674.

32. Emsley P & Cowtan K (2004) Coot: model-building tools for molecular graphics. Acta crystallographica. Section D, Biological crystallography 60(Pt 12 Pt 1):2126-2132.

33. Chen V B, et al. (2010) MolProbity: all-atom structure validation for macromolecular crystallography. Acta crystallographica. Section D, Biological crystallography 66(Pt 1):12-21.

34. Winn M D, et al. (2011) Overview of the CCP4 suite and current developments. Acta crystallographica. Section D, Biological crystallography 67(Pt 4):235-242.

All publications cited herein expressly incorporated herein by reference in their entireties.

What is claimed is:

1) A chimeric epitope comprising:
i. a conserved donor receptor binding site (RBS) or a neutralizing epitope, or a functional fraction thereof; and
ii. an acceptor molecular scaffold or fragment thereof

2) The chimeric epitope of claim 1, wherein the RBS or the neutralizing epitopes are fused to the molecular scaffold.

3) The chimeric epitope of claim 1, wherein the RBS is a conserved epitope on the influenza virus hemagglutinin (HA).

4) The chimeric epitope of claim 1, wherein the acceptor molecular scaffold is an antigenically distinct HA.

5) The chimeric epitope of claim 4, wherein the HA comprises an HA without the RBS or the neutralizing epitopes.

6) The chimeric epitope of claim 1, wherein the donor RBS or the neutralizing epitope and the acceptor molecular scaffold are derived from a family of viruses selected from the group consisting of: Arenaviridae, Bunyaviridae, Coronaviridae, Filoviridae, Flaviviridae, Orthomyxoviridae, Paramyxoviridae, Pneumoviridae, and Retroviridae.

7) The chimeric epitope of claim 1, wherein the donor RBS and the acceptor molecular scaffold are at least one of the following:
a. derived from the family Orthomyxoviridae;
b. derived from the same viral family;
c. derived from a different viral family;
d. derived from the same viral family but different antigenic viral types;
e. derived from the same viral family but different host of origin;
f. derived from the same viral family but different geographical origin;
g. derived from the same viral family but different viral strains or subtypes; and
h. derived from the same viral family but different year of isolation.

8)-14 (canceled)

15) The chimeric epitope of claim 1, wherein the donor RBS is the RBS of circulating, previously circulating, or pre-pandemic influenzas viruses

16) The chimeric epitope of claim 15, wherein the circulating or previously circulating influenzas are H1, H2, H3 or B, and wherein the pre-pandemic influenza viruses are H5, H7 and H9 influenza.

17) (canceled)

18) The chimeric epitope of claim 1, wherein the RBS is an RBS of H1 influenza was isolated in 1918-present day, or the RBS of H1 influenza is H1/Massachusetts/1/1990; H1/Solomon Islands/3/2006; or H1/California/04/2009 or a variant thereof.

19) (canceled)

20) The chimeric epitope of any of claim 1, wherein the molecular scaffold has substantially no preexisting immunity in the population of a subject and/or does not boost a strain-specific response.

21) (canceled)

22) The chimeric epitope of claim 1, wherein the molecular scaffold is derived from group 1, group 2, H2, H4, H5, H6, H7, H8, H9, H10, H11, H12, H13, H14, H15, H16, H17 or H18 influenzas.

23) (canceled)

24) The chimeric epitope of claim 2, wherein group 1 influenza is selected from the group consisting of: H2N2 A/Japan/305/1957; H5N8 A/gyrfalcon/Washington/41088-6/2014; H6N8 A/widgeon/Wisconsin/617/1983; H9N2 A/swine/Hong Kong/9/1998; and H16N3 A/laughing-gull/Delaware Bay/296/1998.

25) The chimeric epitope of claim 22, wherein group 2 influenza is selected from the group consisting of: H3N2 A/Aichi/2/1968; H4N6 A/America black duck/New Brunswick/00464/2010; H7N9 A/Shanghai/1/2013;, H10N7 A/mallard/Wisconsin/1350/1983;
and H14N6 A/mallard/Wisconsin/10OS3941/2010.

26)-29. (canceled)

30) The chimeric epitope of claim 1, wherein the molecular scaffold is engineered to comprise at least one amino acid mutation.

31) The chimeric epitope of claim 30, wherein the at least one amino acid mutation is selected from the group consisting of: N145S, T192R, S193A, K196H, A198E and S219K.

32) An immunogenic composition comprising the chimeric epitope of claim 1 and a pharmaceutically acceptable carrier.

33) The immunogenic composition of claim 32, wherein the composition elicits an immune response in a subject or vaccinates a subject upon administration.

34) (cancelled)

35) The immunogenic composition of claim 32, wherein the composition prevents an infection caused by a pathogen in a subject upon administration.

36) (canceled)

37) (canceled)

38) A method for inducing an immune response in a subject or vaccinating a subject, the method comprising administering to a subject the chimeric epitope of claim 1.

39)-41. (canceled)

42) The method of claim 38, wherein the subject is human, an agricultural or non-domestic animal, a domestic animal, or a bird.

43)-48. (canceled)


SEQUENCE LISTING (173 sequences)

SEQ ID NO: 1 - protein, 217 aa, Influenza virus
APLQLGKCNIAGWILGNPECESLFSKKSWSYIAETPNSENGTCYPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFTPEIAERPKVRGQAGRINYYWTLLEPGDTIIFEANGNLIAPWHAFALNRGFGSG

SEQ ID NO: 2 - protein, 217 aa, Influenza virus
APLQLGNCSIAGWILGNPECESLFSKKSWSYIAETPNSENGTCYPGYFADYEELREQLSSVSSFERFEIFPKESSWPNHTVTKGVTASCSHKGRSSFYRNLLWLTEKNGSYPNLSKSYVNNKEKEVLVLWGVHHPSNIRDQRAIYHTENAYVSVVSSHYNRRFTPEIAKRPKVRDQEGRINYYWTLLEPGDTIIFEANGNLIAPWYAFALSRGFGSG

SEQ ID NO: 3 - protein, 217 aa, Influenza virus
APLQLGNCSIAGWILGNPECESLFSKESWSYIAETPNSENGTCYPGYFADYEELREQLSSVSSFERFEIFPKESSWPNHTVTKGVTASCSHNGKSSFYRNLLWLTEKNGLYPNLSKSYVNNKEKEVLVLWGVHHPSNIGDQRAIYHTENAYVSVVSSHYSRRFTPEIAKRPKVRDQEGRINYYWTLLEPGDTIIFEANGNLIAPWYAFALSRGLGSG

SEQ ID NO: 4 - protein, 217 aa, Influenza virus
APLQLGNCSIAGWILGNPECESLFSKESWSYIAETPDPENGTCYPGYFADYEELREQLSSVSSFERFEIFPKESSWPNHTVTKGVTASCSHNGKSSFYRNLLWLTKKNGLYPNLSKSYVNNKEKEVLVLWGVHHPSNIGDQRAIYHTENAYVSVVSSHYSRRFTPEIAKRPKVRDQEGRINYYWTLLEPGDTIIFEANGNLIAPWYAFALSRGFGSG

SEQ ID NO: 5 - protein, 217 aa, Influenza virus
APLQLGNCSVAGWILGNPECESLFTKESWSYIAETPNPENGTCYPGYFADYEELREQLSSVSSFERFEIFPKESSWPNHTVTKGVTASCSHNGKSSFYRNLLWLTEKNGLYPNLSKSYVNNKEKEVLVLWGVHHPSNMGDQRAIYHTENAYVSVVSSHYSRRFTPEIAKRPKVRDQEGRINYYWTLLEPGDTIIFEANGNLIAPRYAFALSRGFGSG

SEQ ID NO: 6 - protein, 216 aa, Influenza virus
APLQLGNCSVAGWILGNPECESLISKESWSYIVETPNPENGTCYPGYFADYEELREQLSSVSSFERFEIFPKESSWPNHTVTGVTASCSHNGKSSFYRNLLWLTEKNGLYPNLSNSYVNNKEKEVLVLWGVHHPSNIRDQRAIYHTENAYVSVVSSHYSRRFTPEIAKRPKVRGQEGRINYYWTLLEPGDTIIFEANGNLIAPWYAFALSRGFGSG

SEQ ID NO: 7 - protein, 216 aa, Influenza virus
APLQLGNCSVAGWILGNPECELLISKESWSYIVETPNPENGTCYPGYFADYEELREQLSSVSSFERFEIFPKESSWPTHTVTGVSASCSHNGKSSFYRNLLWLTGKNGLYPNLSKSYVNNKEKEVLVLWGVHHPPNIGDQRALYHTENAYVSVVSSHYSRRFTPEIAKRPKVRDQEGRINYYWTLLEPGDTIIFEANGNLIAPWYAFALSRGFGSG

SEQ ID NO: 8 - protein, 216 aa, Influenza virus
APLQLGNCSVAGWILGNPECELLISKESWSYIVETPNPENGTCYPGYFADYEELREQLSSVSSFERFEIFPKESSWPHHTVTGVSASCSHNGKSSFYRNLLWLTGKNGLYPNLSKSYTNNKEKEVLVLWGVHHPPNIGDQRALYHTENAYVSVVSSHYSRRFTPEIAKRPKVRDQEGRINYYWTLLEPGDTIIFEANGNLIAPRYAFALSRGFGSG

SEQ ID NO: 9 - protein, 216 aa, Influenza virus
APLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYANNKEKEVLVLWGVHHPPNIGDQRALYHTENAYVSVVSSHYSRRFTPEIAKRPKVRDQEGRINYYWTLLEPGDTIIFEANGNLIAPRFAFALSRGFGSG

SEQ ID NO: 10 - protein, 216 aa, Influenza virus
APLQLGNCSVAGWILGNPECELLISRESWSYIVEKPNPENGTCYPGHFADYEELREQLSSVSSFERFEIFPKESSWPNHTTTGVSASCSHNGESSFYKNLLWLTGKNGLYPNLSKSYANNKEKEVLVLWGVHHPPNIGDQRALYHTENAYVSVVSSHYSRKFTPEIAKRPKVRDQEGRINYYWTLLEPGDTIIFEANGNLIAPRYAFALSRGFGSG

SEQ ID NO: 11 - protein, 216 aa, Influenza virus
APLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGDQKALYHTENAYVSVVSSHYSRKFTPEIAKRPKVRDQEGRINYYWTLLEPGDTIIFEANGNLIAPRYAFALSRGFGSG

SEQ ID NO: 12 - protein, 217 aa, Influenza virus
APLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSG

SEQ ID NO: 13 - protein, 283 aa, Influenza virus
TNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTDQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKVGTG

SEQ ID NO: 14 - protein, 283 aa, Influenza virus
TNATELVQSSSTGRICDSPHRILDGKNCTLIDALLGDPHCDGFQNEKWDLFVERSKAFSNCYPYDVPDYASLRSLVASSGTLEFINEGFNWTGVTQSGGSYACKRGSDNSFFSRLNWLYESESKYPVLNVTMPNNGNFDKLYIWGVHHPSTDKEQTKLYVRASGRVTVSTKRSQQTIIPNIGSRPWVRGLSSGISIYWTIVKPGDILLINSNGNLIAPRGYFKIRTGKSSIMRSDAPIGTCSSECITPNGSIPNDKPFQNVNKITYGACPKYVKQNTLKLATG

SEQ ID NO: 15 - protein, 283 aa, Influenza virus
TNATELVQSSSTGRICDSPHRILDGKNCTLIDALLGDPHCDGFQNEKWDLFVERSKAFSNCYPYDVPDYASLRSLVASSGTLEFINEGFNWTGVTQSGGSSTCKRGSNNSFFSRLNWLYESESKYPVLNVTMPNNGKFDKLYIWGIHHPSTDKDQTNLYIRASGRVTVSTKRSQQTVIPNIGSRPWVRGLSSRISIYWTIVKPGDILLINSIGNLIAPRGYFKIRTGKSSIMRSDAPIGTCSSECITPNGSIPNDKPFQNVNKITYGACPRYVKQNTLKLATG

SEQ ID NO: 16 - protein, 283 aa, Influenza virus
TNATELVQSSSTGRICDSPHRILDGKNCTLIDALLGDPHCDGFQNEKWDLFIERSKAFSNCYPYDVPDYASLRSLVASSGTLEFINEGFNWTGVTQSGGSYTCKRGSVNSFFSRLNWLYESEYKYPALNVTMPNNGKFDKLYIWGVHHPSTEKEQTNLYVRASGRVTVSTKRSQQTVIPNIGSSPWVRGLSSRISIYWTIVKPGDILLINSTGNLIAPRGYFKIRTGKSSIMRSDAPIGTCSSECITPNGSIPNDKPFQNVNKITYGACPRYVKQNTLKLATG

SEQ ID NO: 17 - protein, 283 aa, Influenza virus
TNATELVQSSSTGRICDSPHRILDGKNCTLIDALLGDPHCDGFQNKEWDLFVERSKAYSNCYPYDVPDYASLRSLVASSGTLEFINEDFNWTGVAQSGESYACKRGSVKSFFSRLNWLHESEYKYPALNVTMPNNGKFDKLYIWGVHHPSTDREQTKLYVRASGRVTVSTKRSQQTVIPNIGSRPWVRGLSSRISIYWTIVKPGDILLINSTGNLIAPRGYFKIRTGKSSIMRSDAPIGTCSSECITPNGSIPNDKPFQNVNRITYGACPRYVKQNTLKLATG

SEQ ID NO: 18 - protein, 283 aa, Influenza virus
TNATELVQSSPTGRICDSPHRILDGKNCTLIDALLGDPHCDGFQNKEWDLFVERSKAYSNCYPYDVPDYASLRSLVASSGTLEFINENFNWTGVAQDGKSYACKRGSVNSFFSRLNWLHKLEYKYPALNVTMPNNGKFDKLYIWGVHHPSTDSDQTSLYVRASGRVTVSTKRSQQTVIPDIGYRPWVRGQSSRISIYWTIVKPGDILLINSTGNLIAPRGYFKIRNGKSSIMRSDAPIGNCSSECITPNGSIPNDKPFQNVNRITYGACPRYVKQNTLKLATG

SEQ ID NO: 19 - protein, 283 aa, Influenza virus
TNATELVQSSSTGGICDSPHQILDGENCTLIDALLGDPQCDGFQNKKWDLFVERSKAYSNCYPYDVPDYASLRSLVASSGTLEFNDESFNWTGVTQNGTSSSCKRRSNNSFFSRLNWLTQLKFKYPALNVTMPNNEKFDKLYIWGVHHPVTDNDQIFLYAQASGRITVSTKRSQQTVIPNIGSRPRIRNIPSRISIYWTIVKPGDILLINSTGNLIAPRGYFKIRSGKSSIMRSDAPIGKCNSECITPNGSIPNDKPFQNVNRITYGACPRYVKQNTLKLATG

SEQ ID NO: 20 - protein, 283 aa, Influenza virus
TNATELVQSSSTGRICDSPHQILDGENCTLIDALLGDPHCDGFQNKEWDLFVERSKAYSNCYPYDVPDYASLRSLVASSGTLEFNNESFNWTGVAQNGTSSACKRRSIKSFFSRLNWLHQLENRYPALNVTMPNNDKFDKLYIWGVHHPSTDSVQTSVYVQASGRVTVSTKRSQQTVIPNIGSRPWVRGVSSRISIYWTIVKPGDILLINSTGNLIAPRGYFKIRSGKSSIMRSDAPIGKCNSECITPNGSIPNDKPFQNVNRITYGACPRYVKQNTLKLATG

SEQ ID NO: 21 - protein, 283 aa, Influenza virus
TNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTRLNFKYPALNVTMPNNEQFDKLYIWGVHHPVTDKEQIFLYAQSSGRITVSTKRSQQAVIPNIGYRPRIRNIPSRISIYWTIVKPGDILLINSTGNLIAPRGYFKIRSGKSSIMRSDAPIGKCNSECITPNGSIPNDKPFQNVNRITYGACPRYVKQSTLKLATG

SEQ ID NO: 22 - protein, 283 aa, Influenza virus
TNATELVQNSSIGEICDSPHQILDGENCTLIDALLGDPQCDGFQNKKWDLFVERSKAYSNCYPYDVPDYASLRSLVASSGTLEFNNESFNWNGVTQNGTSSACIRRSNNSFFSRLNWLTHLNFKYPALNVTMPNNEQFDKLYIWGVHHPVTDKDQIFLYAQPSGRITVSTKRSQQAVIPNIGFRPRIRNIPSRISIYWTIVKPGDILLINSTGNLIAPRGYFKIRSGKSSIMRSDAPIGKCKSECITPNGSIPNDKPFQNVNRITYGACPRYVKQSTLKLATG

SEQ ID NO: 23 - protein, 283 aa, Influenza virus
TNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATG

SEQ ID NO: 24 - protein, 6 aa, Artificial Sequence
  Description of Artificial Sequence Synthetic 6xHis tag
HHHHHH

SEQ ID NO: 25 - protein, 8 aa, Artificial Sequence
  Description of Artificial Sequence Synthetic peptide
WSHPQFEK

SEQ ID NO: 26 - protein, 8 aa, Artificial Sequence
  Description of Artificial Sequence Synthetic peptide
DYKDDDDK

SEQ ID NO: 27 - protein, 8 aa, Influenza virus
KGVTASCS

SEQ ID NO: 28 - protein, 7 aa, Influenza virus
EKNGLYP

SEQ ID NO: 29 - protein, 5 aa, Influenza virus
NIGDQ

SEQ ID NO: 30 - protein, 7 aa, Influenza virus
RDQEGRI

SEQ ID NO: 31 - protein, 7 aa, Influenza virus
GVSASCS

SEQ ID NO: 32 - protein, 7 aa, Influenza virus
GKNGLYP

SEQ ID NO: 33 - protein, 5 aa, Influenza virus
NIGDQ

SEQ ID NO: 34 - protein, 7 aa, Influenza virus
RDQEGRI

SEQ ID NO: 35 - protein, 8 aa, Influenza virus
KGVTAACP

SEQ ID NO: 36 - protein, 7 aa, Influenza virus
KKGNSYP

SEQ ID NO: 37 - protein, 5 aa, Influenza virus
TSADQ

SEQ ID NO: 38 - protein, 7 aa, Influenza virus
RDQEGRM

SEQ ID NO: 39 - protein, 7 aa, Influenza virus
GKSGACK

SEQ ID NO: 40 - protein, 8 aa, Influenza virus
KSDGNAYP

SEQ ID NO: 41 - protein, 5 aa, Influenza virus
TDTEQ

SEQ ID NO: 42 - protein, 7 aa, Influenza virus
RGQSGRI

SEQ ID NO: 43 - protein, 7 aa, Influenza virus
GVTSACL

SEQ ID NO: 44 - protein, 8 aa, Influenza virus
KIKSDPYS

SEQ ID NO: 45 - protein, 5 aa, Influenza virus
NEVEQ

SEQ ID NO: 46 - protein, 7 aa, Influenza virus
NGQRGRI

SEQ ID NO: 47 - protein, 7 aa, Influenza virus
GSSSACL

SEQ ID NO: 48 - protein, 8 aa, Influenza virus
RVKNGNYG

SEQ ID NO: 49 - protein, 5 aa, Influenza virus
SDTEQ

SEQ ID NO: 50 - protein, 7 aa, Influenza virus
RNQSGRI

SEQ ID NO: 51 - protein, 7 aa, Influenza virus
GVTSACL

SEQ ID NO: 52 - protein, 7 aa, Influenza virus
KQNDRYP

SEQ ID NO: 53 - protein, 5 aa, Influenza virus
TEATA

SEQ ID NO: 54 - protein, 7 aa, Influenza virus
EGQRGWM

SEQ ID NO: 55 - protein, 8 aa, Influenza virus
RGVTASCS

SEQ ID NO: 56 - protein, 7 aa, Influenza virus
EKNGSYP

SEQ ID NO: 57 - protein, 5 aa, Influenza virus
NIEDQ

SEQ ID NO: 58 - protein, 7 aa, Influenza virus
RGQAGRI

SEQ ID NO: 59 - protein, 8 aa, Influenza virus
KGVTASCS

SEQ ID NO: 60 - protein, 7 aa, Influenza virus
KKNGSYP

SEQ ID NO: 61 - protein, 5 aa, Influenza virus
NIGDQ

SEQ ID NO: 62 - protein, 7 aa, Influenza virus
RDQEGRI

SEQ ID NO: 63 - protein, 8 aa, Influenza virus
KGVTASCS

SEQ ID NO: 64 - protein, 7 aa, Influenza virus
EKNGSYP

SEQ ID NO: 65 - protein, 5 aa, Influenza virus
NIRDQ

SEQ ID NO: 66 - protein, 7 aa, Influenza virus
RDQEGRI

SEQ ID NO: 67 - protein, 8 aa, Influenza virus
KGVTASCS

SEQ ID NO: 68 - protein, 7 aa, Influenza virus
KKNGSYP

SEQ ID NO: 69 - protein, 5 aa, Influenza virus
NIGDQ

SEQ ID NO: 70 - protein, 7 aa, Influenza virus
RGQEGRI

SEQ ID NO: 71 - protein, 8 aa, Influenza virus
KGVTASCS

SEQ ID NO: 72 - protein, 7 aa, Influenza virus
KKNGLYP

SEQ ID NO: 73 - protein, 5 aa, Influenza virus
NIGDQ

SEQ ID NO: 74 - protein, 7 aa, Influenza virus
RDQEGRI

SEQ ID NO: 75 - protein, 8 aa, Influenza virus
KGVTASCS

SEQ ID NO: 76 - protein, 7 aa, Influenza virus
EKNGLYP

SEQ ID NO: 77 - protein, 5 aa, Influenza virus
NMGDQ

SEQ ID NO: 78 - protein, 7 aa, Influenza virus
RDQEGRI

SEQ ID NO: 79 - protein, 7 aa, Influenza virus
GVTASCS

SEQ ID NO: 80 - protein, 7 aa, Influenza virus
EKNGLYP

SEQ ID NO: 81 - protein, 5 aa, Influenza virus
NIRDQ

SEQ ID NO: 82 - protein, 7 aa, Influenza virus
RGQEGRI

SEQ ID NO: 83 - protein, 7 aa, Influenza virus
GVSASCS

SEQ ID NO: 84 - protein, 7 aa, Influenza virus
GKNGLYP

SEQ ID NO: 85 - protein, 5 aa, Influenza virus
NIGDQ

SEQ ID NO: 86 - protein, 7 aa, Influenza virus
RDQEGRI

SEQ ID NO: 87 - protein, 7 aa, Influenza virus
GVSASCS

SEQ ID NO: 88 - protein, 7 aa, Influenza virus
GKNGLYP

SEQ ID NO: 89 - protein, 5 aa, Influenza virus
NIGDQ

SEQ ID NO: 90 - protein, 7 aa, Influenza virus
RDQEGRI

SEQ ID NO: 91 - protein, 7 aa, Influenza virus
GVSASCS

SEQ ID NO: 92 - protein, 7 aa, Influenza virus
GKNGLYP

SEQ ID NO: 93 - protein, 5 aa, Influenza virus
NIGDQ

SEQ ID NO: 94 - protein, 7 aa, Influenza virus
RDQEGRI

SEQ ID NO: 95 - protein, 7 aa, Influenza virus
GVSASCS

SEQ ID NO: 96 - protein, 7 aa, Influenza virus
GKNGLYP

SEQ ID NO: 97 - protein, 5 aa, Influenza virus
NIGDQ

SEQ ID NO: 98 - protein, 7 aa, Influenza virus
RDQEGRI

SEQ ID NO: 99 - protein, 7 aa, Influenza virus
GVSASCS

SEQ ID NO: 100 - protein, 7 aa, Influenza virus
GKNGLYP

SEQ ID NO: 101 - protein, 5 aa, Influenza virus
NIGDQ

SEQ ID NO: 102 - protein, 7 aa, Influenza virus
RDQEGRI

SEQ ID NO: 103 - protein, 8 aa, Influenza virus
KGVTAACP

SEQ ID NO: 104 - protein, 7 aa, Influenza virus
KKGNSYP

SEQ ID NO: 105 - protein, 5 aa, Influenza virus
TTADQ

SEQ ID NO: 106 - protein, 7 aa, Influenza virus
RDQEGRM

SEQ ID NO: 107 - protein, 8 aa, Influenza virus
KGVTAACP

SEQ ID NO: 108 - protein, 7 aa, Influenza virus
KKGNSYP

SEQ ID NO: 109 - protein, 5 aa, Influenza virus
TSTDQ

SEQ ID NO: 110 - protein, 7 aa, Influenza virus
RDQEGRM

SEQ ID NO: 111 - protein, 8 aa, Influenza virus
KGVTAACP

SEQ ID NO: 112 - protein, 7 aa, Influenza virus
KKGNSYP

SEQ ID NO: 113 - protein, 5 aa, Influenza virus
TTADQ

SEQ ID NO: 114 - protein, 7 aa, Influenza virus
RDQEGRM

SEQ ID NO: 115 - protein, 8 aa, Influenza virus
KGVTAACP

SEQ ID NO: 116 - protein, 7 aa, Influenza virus
KKGNSYP

SEQ ID NO: 117 - protein, 5 aa, Influenza virus
TTADQ

SEQ ID NO: 118 - protein, 7 aa, Influenza virus
RDQEGRM

SEQ ID NO: 119 - protein, 8 aa, Influenza virus
KGVTAACP

SEQ ID NO: 120 - protein, 7 aa, Influenza virus
KKGNSYP

SEQ ID NO: 121 - protein, 5 aa, Influenza virus
TTADQ

SEQ ID NO: 122 - protein, 7 aa, Influenza virus
RDQEGRM

SEQ ID NO: 123 - protein, 7 aa, Influenza virus
GGSRACA

SEQ ID NO: 124 - protein, 7 aa, Influenza virus
EKGSNYP

SEQ ID NO: 125 - protein, 5 aa, Influenza virus
DETEQ

SEQ ID NO: 126 - protein, 7 aa, Influenza virus
NGLGSRM

SEQ ID NO: 127 - protein, 8 aa, Influenza virus
LGVSSACP

SEQ ID NO: 128 - protein, 7 aa, Influenza virus
KKNSTYP

SEQ ID NO: 129 - protein, 5 aa, Influenza virus
DAAEQ

SEQ ID NO: 130 - protein, 7 aa, Influenza virus
NGQSGRI

SEQ ID NO: 131 - protein, 22 aa, Artificial Sequence
  Description of Artificial Sequence Synthetic peptide
CARYRGHVVTVENYYYYMDVWG

SEQ ID NO: 132 - protein, 22 aa, Artificial Sequence
  Description of Artificial Sequence Synthetic peptide
CARERTELVTVDYYYSYMDVWG

SEQ ID NO: 133 - protein, 22 aa, Artificial Sequence
  Description of Artificial Sequence Synthetic peptide
CAREVTDFVTVDYIYYYIDVWG

SEQ ID NO: 134 - protein, 22 aa, Artificial Sequence
  Description of Artificial Sequence Synthetic peptide
CARRRSDFESVDYIYHYMDVWG

SEQ ID NO: 135 - protein, 22 aa, Artificial Sequence
  Description of Artificial Sequence Synthetic peptide
CARTFSEVLTVDYIYYYMDVWG

SEQ ID NO: 136 - protein, 22 aa, Artificial Sequence
  Description of Artificial Sequence Synthetic peptide
CARRRHELVTVDYYYYFMDVWG

SEQ ID NO: 137 - protein, 22 aa, Artificial Sequence
  Description of Artificial Sequence Synthetic peptide
CARDKGTVVTVDYYYYSMDVWG

SEQ ID NO: 138 - protein, 20 aa, Artificial Sequence
  Description of Artificial Sequence Synthetic peptide
CAREAYYDVDKYYHYMDVWG

SEQ ID NO: 139 - protein, 22 aa, Artificial Sequence
  Description of Artificial Sequence Synthetic peptide
CARGPFGVVTVDYSYYYMDVWG

SEQ ID NO: 140 - protein, 22 aa, Artificial Sequence
  Description of Artificial Sequence Synthetic peptide
CARGGLEPRSVDYYYYGMDVWG

SEQ ID NO: 141 - protein, 22 aa, Artificial Sequence
  Description of Artificial Sequence Synthetic peptide
CARLKSDFSTVDHIYYYFDVWG

SEQ ID NO: 142 - protein, 21 aa, Artificial Sequence
  Description of Artificial Sequence Synthetic peptide
CARAVVPATVDYYYYYIDVWG

SEQ ID NO: 143 - protein, 21 aa, Artificial Sequence
  Description of Artificial Sequence Synthetic peptide
CARERVPASVDYYFYYMDVWG

SEQ ID NO: 144 - protein, 23 aa, Artificial Sequence
  Description of Artificial Sequence Synthetic peptide
CAKDFRSSSSMDYFYYYGMGVWG

SEQ ID NO: 145 - protein, 22 aa, Artificial Sequence
  Description of Artificial Sequence Synthetic peptide
CARAMTVLETVDYYYYVMDVWG

SEQ ID NO: 146 - protein, 22 aa, Artificial Sequence
  Description of Artificial Sequence Synthetic peptide
CARIKTTLITVEFYYYDMDVWG

SEQ ID NO: 147 - protein, 22 aa, Artificial Sequence
  Description of Artificial Sequence Synthetic peptide
CARYKGTVVTVEYYYYYMDVWG

SEQ ID NO: 148 - protein, 21 aa, Artificial Sequence
  Description of Artificial Sequence Synthetic peptide
CVRVVVPASIENYYYYIDVWG

SEQ ID NO: 149 - protein, 22 aa, Artificial Sequence
  Description of Artificial Sequence Synthetic peptide
CARDRGDVVTTDYYYYYMDVWG

SEQ ID NO: 150 - protein, 22 aa, Artificial Sequence
  Description of Artificial Sequence Synthetic peptide
CARWRSEVATVDYIYHYMDVWG

SEQ ID NO: 151 - protein, 22 aa, Artificial Sequence
  Description of Artificial Sequence Synthetic peptide
CARFISELVTVDHYFYYMDVWG

SEQ ID NO: 152 - protein, 26 aa, Artificial Sequence
  Description of Artificial Sequence Synthetic peptide
CARAPPYCTSASCPDDYYYFYMDVWG

SEQ ID NO: 153 - protein, 22 aa, Artificial Sequence
  Description of Artificial Sequence Synthetic peptide
CAREVSDFITVDYIYYYIDVWG

SEQ ID NO: 154 - protein, 22 aa, Artificial Sequence
  Description of Artificial Sequence Synthetic peptide
CARTFSEVLTVDYIYYYMDVWG

SEQ ID NO: 155 - protein, 22 aa, Artificial Sequence
  Description of Artificial Sequence Synthetic peptide
CARDKGTVVTVDYYYYNMDVWG

SEQ ID NO: 156 - protein, 22 aa, Artificial Sequence
  Description of Artificial Sequence Synthetic peptide
CARDVGHIFTVDSYFYYIDVWG

SEQ ID NO: 157 - protein, 22 aa, Artificial Sequence
  Description of Artificial Sequence Synthetic peptide
CAREASAYDSVDSVYYYFDVWG

SEQ ID NO: 158 - protein, 23 aa, Artificial Sequence
  Description of Artificial Sequence Synthetic peptide
CARLRTELITVDAWYYYGMDVWG

SEQ ID NO: 159 - protein, 23 aa, Artificial Sequence
  Description of Artificial Sequence Synthetic peptide
CAREAGGLLTSVDYYYYSMDVWG

SEQ ID NO: 160 - protein, 22 aa, Artificial Sequence
  Description of Artificial Sequence Synthetic peptide
CARLKTDFDSVDYINYFMDVWG

SEQ ID NO: 161 - protein, 22 aa, Artificial Sequence
  Description of Artificial Sequence Synthetic peptide
CARRRHELVTVEYYYYFMDVWG

SEQ ID NO: 162 - protein, 22 aa, Artificial Sequence
  Description of Artificial Sequence Synthetic peptide
CARAASTLVTIDYYYYSMDVWG

SEQ ID NO: 163 - protein, 21 aa, Artificial Sequence
  Description of Artificial Sequence Synthetic peptide
CARVVVPVDIDYYYYYMDVWG

SEQ ID NO: 164 - protein, 23 aa, Artificial Sequence
  Description of Artificial Sequence Synthetic peptide
CAREATIFGVSIEYYYYIMDVWG

SEQ ID NO: 165 - protein, 22 aa, Artificial Sequence
  Description of Artificial Sequence Synthetic peptide
CARERVIVETIESFYYYMDVWG

SEQ ID NO: 166 - protein, 22 aa, Artificial Sequence
  Description of Artificial Sequence Synthetic peptide
CARERVIVATIESFYYYMDVWG

SEQ ID NO: 167 - protein, 23 aa, Artificial Sequence
  Description of Artificial Sequence Synthetic peptide
CAKDFRTSSSMDYYYFYGMGVWG

SEQ ID NO: 168 - protein, 20 aa, Artificial Sequence
  Description of Artificial Sequence Synthetic peptide
CARDYRAAGLDYFYYLDVWG

SEQ ID NO: 169 - protein, 23 aa, Artificial Sequence
  Description of Artificial Sequence Synthetic peptide
CAKDRVDTTSWDYYFHKSMDVWG

SEQ ID NO: 170 - protein, 23 aa, Artificial Sequence
  Description of Artificial Sequence Synthetic peptide
CAKDLRASSSMDYYYYSGMDVWG

SEQ ID NO: 171 - protein, 24 aa, Artificial Sequence
  Description of Artificial Sequence Synthetic peptide
CAREIPGATSGPDHFFFYGMDVWG

SEQ ID NO: 172 - protein, 24 aa, Artificial Sequence
  Description of Artificial Sequence Synthetic peptide
CVRGVREVGATGGDPFYYAMAVWG

SEQ ID NO: 173 - protein, 5 aa, Influenza virus
SDEDQ